SEQUENCE LISTING (46 sequences)

SEQ ID NO: 1 - DNA, 18 bp, Artificial Sequence
  Synthetic
cggtggacaaattgtcac

SEQ ID NO: 2 - DNA, 22 bp, Artificial Sequence
  Synthetic
cttctctggatttaacacactt

SEQ ID NO: 3 - DNA, 28 bp, Artificial Sequence
  Synthetic
ttacaagcttaaagaatgtctgaacact

SEQ ID NO: 4 - DNA, 27 bp, Artificial Sequence
  Synthetic
ttgaatttaggtgaaacatttgtcacg

SEQ ID NO: 5 - DNA, 51 bp, Artificial Sequence
  Synthetic
tcagcacacaaagccaaaaatttatttttctgtgcaaaggaaattaaggag

SEQ ID NO: 6 - DNA, 49 bp, Artificial Sequence
  Synthetic
tattggtggagctaaacttaaagccttttctgtacaatccctttgagtg

SEQ ID NO: 7 - DNA, 47 bp, Artificial Sequence
  Synthetic
tcagcacacaaagccaaaaatttatctgtgcaaaggaaattaaggag

SEQ ID NO: 8 - DNA, 45 bp, Artificial Sequence
  Synthetic
tattggtggagctaaacttaaagccctgtacaatccctttgagtg

SEQ ID NO: 9 - DNA, 18 bp, Artificial Sequence
  Synthetic
tgcttcagtcagctgatg

SEQ ID NO: 10 - DNA, 22 bp, Artificial Sequence
  Synthetic
ttaaattgtcatcttcgtcctt

SEQ ID NO: 11 - DNA, 41 bp, Artificial Sequence
  Synthetic
tcagtactagtgcctgtgcccacaatcgtttttaaacgggt

SEQ ID NO: 12 - DNA, 43 bp, Artificial Sequence
  Synthetic
tcgtatacagggcttttgacatctatcttggaagcgacaacaa

SEQ ID NO: 13 - DNA, 17 bp, Artificial Sequence
  Synthetic
ctgcacttacaccgcaa

SEQ ID NO: 14 - DNA, 22 bp, Artificial Sequence
  Synthetic
gtagctggttttgctaaattcc

SEQ ID NO: 15 - DNA, 17 bp, Artificial Sequence
  Synthetic
ttgatgagctggagcca

SEQ ID NO: 16 - DNA, 18 bp, Artificial Sequence
  Synthetic
caccctcaatgcagagtc

SEQ ID NO: 17 - DNA, 41 bp, Artificial Sequence
  Synthetic
gtgtkaccctgaagactcggttttagccactgactcggatc

SEQ ID NO: 18 - DNA, 45 bp, Artificial Sequence
  Synthetic
cctccgtgatatggctcttcgtttttttcttacatggctctggtc

SEQ ID NO: 19 - DNA, 20 bp, Artificial Sequence
  Synthetic
atgtggatggctgagttgtt

SEQ ID NO: 20 - DNA, 20 bp, Artificial Sequence
  Synthetic
catgctgagtactggacctc

SEQ ID NO: 21 - DNA, 13 bp, Artificial Sequence
  Synthetic
cagccatccacat

SEQ ID NO: 22 - protein, 8 aa, Artificial Sequence
  Synthetic
DYKDDDDK

SEQ ID NO: 23 - protein, 6 aa, Artificial Sequence
  Synthetic
HHHHHH

SEQ ID NO: 24 - protein, 9 aa, Artificial Sequence
  Synthetic
YPYDVPDYA

SEQ ID NO: 25 - protein, 10 aa, Artificial Sequence
  Synthetic
EQKLISEEDL

SEQ ID NO: 26 - DNA, 30 bp, Artificial Sequence
  Synthetic
gtactgccactaaagcatacaatgtaacac

SEQ ID NO: 27 - DNA, 31 bp, Artificial Sequence
  Synthetic
aatatgcttattcagcaaaatgacttgatct

SEQ ID NO: 28 - DNA, 48 bp, Artificial Sequence
  Synthetic
  misc_feature (33)..(34): may be modified by dSpacer
cagacaaggaactgattacaaacattggccgcaattgcacaatttgcc

SEQ ID NO: 29 - DNA, 497 bp, Artificial Sequence
  Synthetic
ggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccacc

SEQ ID NO: 30 - DNA, 494 bp, Artificial Sequence
  Synthetic
ggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgaacaagcacattgacgcatacaaaacattcccacc

SEQ ID NO: 31 - DNA, 30 bp, Artificial Sequence
  Synthetic
tctgataatggaccccaaaatcagcgaaat

SEQ ID NO: 32 - DNA, 30 bp, Artificial Sequence
  Synthetic
ctccattctggttactgccagttgaatctg

SEQ ID NO: 33 - DNA, 30 bp, Artificial Sequence
  Synthetic
gcaactgagggagccttgaatacaccaaaa

SEQ ID NO: 34 - DNA, 30 bp, Artificial Sequence
  Synthetic
tgaggaagttgtagcacgattgcagcattg

SEQ ID NO: 35 - DNA, 30 bp, Artificial Sequence
  Synthetic
aaggaactgattacaaacattggccgcaaa

SEQ ID NO: 36 - DNA, 30 bp, Artificial Sequence
  Synthetic
ttccatgccaatgcgcgacattccgaagaa

SEQ ID NO: 37 - DNA, 30 bp, Artificial Sequence
  Synthetic
aaattttggggaccaggaactaatcagaca

SEQ ID NO: 38 - DNA, 30 bp, Artificial Sequence
  Synthetic
tggcacctgtgtaggtcaaccacgttcccg

SEQ ID NO: 39 - DNA, 32 bp, Artificial Sequence
  Synthetic
accccaaaatcagcgaaatgcaccccgcatta

SEQ ID NO: 40 - DNA, 25 bp, Artificial Sequence
  Synthetic
gtagaaataccatcttggactgaga

SEQ ID NO: 41 - DNA, 28 bp, Artificial Sequence
  Synthetic
gtctgataatggaccccaaaatcagcga

SEQ ID NO: 42 - DNA, 32 bp, Artificial Sequence
  Synthetic
tagtagaaataccatcttggactgagatcttt

SEQ ID NO: 43 - DNA, 45 bp, Artificial Sequence
  Synthetic
agaatggagaacgcagtggggcgcgatcaaaacaacgtcggcccc

SEQ ID NO: 44 - DNA, 44 bp, Artificial Sequence
  Synthetic
  misc_feature (29)..(30): may be modified by dSpacer
agaatggagaacgcagtggggcgcgatcaaacaacgtcggcccc

SEQ ID NO: 45 - DNA, 50 bp, Artificial Sequence
  Synthetic
cagtaaccagaatggagaacgcagtggggcgcgatcaaaacaacgtcggc

SEQ ID NO: 46 - DNA, 50 bp, Artificial Sequence
  Synthetic
  misc_feature (37)..(38): may be modified by dSpacer
cagtaaccagaatggagaacgcagtggggcgcgatcaaacaacgtcggcc

(19) United States
(12) Patent Application Publication
(10) Pub. No.: US 2021/0292825 A1
(43) Pub. Date: Sep. 23, 2021

(54) RAPID DIAGNOSTIC TEST

(71) Applicant: Detect, Inc., Guilford, CT (US)

(72) Inventors: Jonathan M. Rothberg, Guilford, CT (US); Spencer Glantz, West Hartford, CT (US); Benjamin Rosenbluth, Hamden, CT (US); Matthew Dyer, Spring, TX (US); Todd Roswech, Ivoryton, CT (US); Eric Kauderer-Abrams, Redwood City, CA (US); Jose Camara, Saratoga, CA (US)

(73) Assignee: Detect, Inc., Guilford, CT (US)

(21) Appl. No.: 17/203,724

(22) Filed: Mar. 16, 2021

Related U.S. Application Data
(60) Provisional application No. 63/091,768, filed on Oct. 14, 2020.
(60) Provisional application No. 63/081,201, filed on Sep. 21, 2020.
(60) Provisional application No. 63/074,524, filed on Sep. 4, 2020.
(60) Provisional application No. 63/068,303, filed on Aug. 20, 2020.
(60) Provisional application No. 63/066,770, filed on Aug. 17, 2020.
(60) Provisional application No. 63/066,111, filed on Aug. 14, 2020.
(60) Provisional application No. 63/065,131, filed on Aug. 13, 2020.
(60) Provisional application No. 63/061,072, filed on Aug. 4, 2020.
(60) Provisional application No. 63/059,928, filed on Jul. 31, 2020.
(60) Provisional application No. 63/053,534, filed on Jul. 17, 2020.
(60) Provisional application No. 63/036,887, filed on Jun. 9, 2020.
(60) Provisional application No. 63/034,901, filed on Jun. 4, 2020.
(60) Provisional application No. 63/027,859, filed on May 20, 2020.
(60) Provisional application No. 63/027,874, filed on May 20, 2020.
(60) Provisional application No. 63/027,890, filed on May 20, 2020.
(60) Provisional application No. 63/027,864, filed on May 20, 2020.
(60) Provisional application No. 63/027,878, filed on May 20, 2020.
(60) Provisional application No. 63/027,886, filed on May 20, 2020.
(60) Provisional application No. 63/022,534, filed on May 10, 2020.
(60) Provisional application No. 63/022,533, filed on May 10, 2020.
(60) Provisional application No. 63/016,797, filed on Apr. 28, 2020.
(60) Provisional application No. 63/013,450, filed on Apr. 21, 2020.
(60) Provisional application No. 63/010,578, filed on Apr. 15, 2020.
(60) Provisional application No. 63/010,626, filed on Apr. 15, 2020.
(60) Provisional application No. 63/002,209, filed on Mar. 30, 2020.
(60) Provisional application No. 62/991,039, filed on Mar. 17, 2020.

(51) Int. Cl. C12Q 1/6844; C12Q 1/6806

(52) U.S. Cl. CPC C12Q 1/6844; C12Q 1/6806

(57) ABSTRACT

Provided herein are rapid diagnostic tests, systems, and methods for detecting one or more target nucleic acid sequences (e.g., a nucleic acid sequence of one or more pathogens, such as SARS-CoV-2 or an influenza virus) using isothermal nucleic acid amplification. The tests, systems, and methods described herein may be performed in a point-of-care setting or a home setting without specialized equipment.

### RELATED APPLICATIONS

The present application claims the benefit under 35 U.S.C. § 119(e) of U.S. Provisional Patent Application No. 62/991,039, filed Mar. 17, 2020 and titled “Viral Rapid Test”; U.S. Provisional Patent Application No. 63/002,209, filed Mar. 30, 2020 and titled “Viral Rapid Test”; U.S. Provisional Patent Application No. 63/010,578, filed Apr. 15, 2020 and titled “Viral Rapid Test”; U.S. Provisional Patent Application No. 63/010,626, filed Apr. 15, 2020 and titled “Viral Rapid Colorimetric Test”; U.S. Provisional Patent Application No. 63/013,450, filed Apr. 21, 2020 and titled “Method of Making and Using a Viral Test Kit”; U.S. Provisional Patent Application No. 63/016,797, filed Apr. 28, 2020 and titled “Sample Swab with Built-In Illness Test”; U.S. Provisional Patent Application No. 63/022,533, filed May 10, 2020 and titled “Rapid Diagnostic Test”; U.S. Provisional Patent Application No. 63/022,534, filed May 10, 2020 and titled “Rapid Diagnostic Test”; U.S. Provisional Patent Application No. 63/027,859, filed May 20, 2020 and titled “Rapid Self Administrable Test”; U.S. Provisional Patent Application No. 63/027,864, filed May 20, 2020 and titled “Rapid Self Administrable Test”; U.S. Provisional Patent Application No. 63/027,874, filed May 20, 2020 and titled “Rapid Self Administrable Test”; U.S. Provisional Patent Application No. 63/027,878, filed May 20, 2020 and titled “Rapid Self Administrable Test”; U.S. Provisional Patent Application No. 63/027,886, filed May 20, 2020 and titled “Rapid Self Administrable Test”; U.S. Provisional Patent Application No. 63/027,890, filed May 20, 2020 and titled “Rapid Self Administrable Test”; U.S. Provisional Patent Application No. 63/034,901, filed Jun. 4, 2020 and titled “Breakable Sample Collection Swab”; U.S. Provisional Patent Application No. 63/036,887, filed Jun. 9, 2020 and titled “Rapid Diagnostic Test”; U.S. Provisional Patent Application No. 63/053,534, filed Jul. 17, 2020 and titled “Computer Vision Algorithm For Diagnostic Testing”; U.S. Provisional Patent Application No. 63/059,928, filed Jul. 31, 2020 and titled “Rapid Diagnostic Test”; U.S. Provisional Patent Application No. 63/061,072, filed Aug. 4, 2020 and titled “Rapid Diagnostic Test”; U.S. Provisional Patent Application No. 63/065,131, filed Aug. 13, 2020 and titled “Apparatuses and Methods for Performing Rapid Diagnostic Tests”; U.S. Provisional Patent Application No. 63/066,111, filed Aug. 14, 2020 and titled “Apparatuses and Methods for Performing Rapid Diagnostic Tests”; U.S. Provisional Patent Application No. 63/066,770, filed Aug. 17, 2020 and titled “Apparatuses and Methods for Performing Rapid Diagnostic Tests”; U.S. Provisional Patent Application No. 63/068,303, filed Aug. 20, 2020 and titled “Apparatuses and Methods for Performing Rapid Multiplexed Diagnostic Tests”; U.S. Provisional Patent Application No. 63/074,524, filed Sep. 4, 2020 and titled “Rapid Diagnostic Test with Integrated Swab”; U.S. Provisional Patent Application No. 63/081,201, filed Sep. 21, 2020 and titled “Rapid Diagnostic Test”; and U.S. Provisional Patent Application No. 63/091,768, filed Oct. 14, 2020 and titled “Rapid Diagnostic Test,” each of which is hereby incorporated by reference in its entirety.

### REFERENCE TO SEQUENCE LISTING SUBMITTED AS A TEXT FILE VIA EFS-WEB

The present application contains a sequence listing which has been submitted in ASCII format via EFS-Web and is hereby incorporated by reference in its entirety. Said ASCII copy, created on Mar. 16, 2021 is named H096670033US00-SEQ-RJP and is 11 kilobytes in size.

### FIELD

The present invention generally relates to diagnostic tests, systems, and methods for detecting the presence of a target nucleic acid sequence.

### BACKGROUND

The ability to rapidly diagnose disease is critical to preserving human health. Fast, reliable testing options which can be easily- or self-administered are important both to expand access to healthcare and to reduce unnecessary human-to-human interaction in the face of highly infectious diseases. As one example, the lack of adequate testing materials and lack of access to diagnostic tests for the highly-contagious and highly-lethal, yet often asymptomatic, coronavirus disease 2019 (COVID-19) has played a critical role in a global pandemic that has infected millions and killed hundreds of thousands of people. Undiagnosed patients became “super spreaders,” and unreliable data on the infectivity rate of the virus delayed vaccine rollout and governmental response, leading to unnecessary harm worldwide. The existence of a rapid, accurate diagnostic test could allow infected individuals to be quickly identified and isolated, which could facilitate containment of disease and treatment of infected individuals.

### SUMMARY

Provided herein are a number of diagnostic tests useful for detecting target nucleic acid sequences. The tests, as described herein, are able to be performed in a point-of-care (POC) setting or home setting without specialized equipment.

In some aspects, a diagnostic system is provided. In some embodiments, the diagnostic system comprises a sample-collecting component. In some embodiments, the diagnostic system comprises one or more nucleic acid amplification reagents. In certain embodiments, the one or more nucleic acid amplification reagents comprise a first primer directed to a first target nucleic acid and labeled with a first label. In some embodiments, the diagnostic system comprises a readout device configured to detect the presence of the first target nucleic acid.

In some aspects, a diagnostic system is provided. In some embodiments, the diagnostic system comprises a sample-collecting component configured to collect a sample. In some embodiments, the diagnostic system comprises one or more isothermal nucleic acid amplification reagents. In certain embodiments, the one or more isothermal nucleic acid amplification reagents comprise a first primer directed to a first target nucleic acid. In some embodiments, the diagnostic system comprises a readout device configured to detect the first target nucleic acid when the concentration of the first target nucleic acid in the sample is about 5 genomic copies per μL or more.

In some aspects, a diagnostic method is provided. In some embodiments, the diagnostic method comprises collecting a sample from a subject. In some embodiments, the diagnostic method comprises performing an isothermal nucleic acid amplification reaction configured to amplify a first target nucleic acid. In some embodiments, the diagnostic method comprises detecting the presence or absence of the first target nucleic acid in the sample within 75 minutes.

### BRIEF DESCRIPTION OF THE DRAWINGS

FIG. 1A is, according to some embodiments, a schematic illustration of an exemplary diagnostic system comprising a sample-collecting component, a first reaction tube comprising a first cap and a second cap, a heater, and a readout device;

FIG. 1B is, according to some embodiments, a schematic illustration of an exemplary diagnostic system comprising a sample-collecting component, a first reaction tube comprising a first cap, a second cap, and a third cap, a heater, and a readout device;

FIG. 2 is a photograph of an exemplary diagnostic system comprising a first sample-collecting component, a second sample-collecting component, a first reaction tube, a second reaction tube, a cap comprising one or more reagents, a pipette, a dropper, a heater, and a readout device, according to some embodiments;

FIG. 3 is, according to some embodiments, a schematic illustration of an exemplary reverse-transcription loop-mediated isothermal amplification (RT-LAMP) method;

FIG. 4 shows exemplary haptens that may be used in a dual-hapten labeling probe, according to some embodiments;

FIG. 5A is, according to some embodiments, a schematic depicting a direct recombinase polymerase amplification (RPA) method;

FIG. 5B is, according to some embodiments, a schematic depicting RT-RPA to detect the N gene of SARS-CoV-2;

FIG. 6 is a schematic depicting lateral flow technology with gold particles, according to some embodiments;

FIG. 7A is, according to some embodiments, a schematic illustration of a lateral flow strip comprising a test line and two control lines prior to being contacted with a sample;

FIG. 7B is, according to some embodiments, a schematic illustration of a lateral flow strip comprising a test line and two control lines after being contacted with a sample;

FIG. 8 is a schematic illustrating positive and negative test results on a lateral flow strip, according to some embodiments;

FIGS. 9A-9E are, according to some embodiments, schematic illustrations of lateral flow assay strips usable for multiplexed testing;

FIG. 10 is a schematic illustration of a colorimetric device, according to some embodiments;

FIGS. 11A-11D show, according to some embodiments, screenshots from a downloadable software application;

FIG. 12 is, according to some embodiments, a schematic illustration of an exemplary readout device comprising a chimney;

FIGS. 13A-13C are schematic illustrations of a caged cap, according to some embodiments;

FIG. 14 is, according to some embodiments, a schematic illustration of the components of an exemplary rapid diagnostic system comprising two nasal swabs, a collection tube, a warmer tube, a tube rack, a pipette, a test cap, a dropper, a readout device, a warmer, a results card, serial number stickers, personal health information stickers, a negative control, and a positive control;

FIG. 15A-15H are schematic illustrations of the steps of an exemplary diagnostic method, according to some embodiments;

FIG. 16 is, according to some embodiments, a schematic illustration of a lateral flow strip readout with positive and negative results;

FIG. 17 is a table showing different types of controls and their uses in monitoring, along with the expected readout appearance on a lateral flow assay strip, according to some embodiments;

FIG. 18A is, according to some embodiments, a bar graph of the performance of an exemplary rapid diagnostic kit as compared to the CDC 2019 novel coronavirus (2019-nCoV) RT-PCR diagnostic panel;

FIG. 18B is, according to some embodiments, a bar graph of the performance of an exemplary rapid diagnostic kit as compared to the Roche cobas SARS-CoV-2 RT-PCR test;

FIG. 19A is a schematic illustration of a “No Flow” invalid result, where no bands are visible on the lateral flow strip;

FIG. 19B is a schematic illustration of a “No Sample Processing Control” invalid result, where only 1 band (i.e., the Readout Check Control) was visible on the lateral flow strip;

FIG. 20 shows an alignment between SARS-CoV-2 (upper sequence) and SARS-CoV-1 (lower sequence). The underlined portions show the three candidate regions for potential primer-binding sites;

FIG. 21A is a photograph of five test strips of various saliva concentrations of an input sample, demonstrating that recombinase polymerase amplification (RPA) is tolerant across the entire range (0%-30%);

FIG. 21B is a photograph of test strips from various temperature experiments, demonstrating that different body temperatures (hand, front pant pocket, rear pant pocket) were all sufficient to drive an RPA reaction;

FIG. 22 is a photograph of a series of test strips detecting multiple concentrations of COVID-19 DNA;

FIG. 23 is a schematic illustrating the laboratory workflow of a test embodiment;

FIG. 24 is a series of photographs of lateral flow tests showing test results following use of different concentrations of UDG and dUTP during processing;

FIG. 25 is a photograph of a series of test strips after administration of different concentrations of SARS-CoV-2 RNA; and

FIG. 26 is a photograph of a series of colorimetric RT-LAMP titration reactions used to detect approximately 1 fM SARS-CoV-2 RNA in solution;

### DETAILED DESCRIPTION

The present disclosure provides diagnostic tests, systems, and methods for rapidly detecting one or more target nucleic acid sequences. Such target nucleic acid sequences may, in some embodiments, be a nucleic acid sequence of a pathogen, such as SARS-CoV-2, an influenza virus, or any other pathogen (e.g., a virus, bacterium, protozoan, prion, viroid, parasite, fungus). The diagnostic tests, systems, and methods described herein utilize methods of isothermal nucleic acid amplification and are capable of producing highly accurate results (e.g., as accurate as PCR-based methods of detection) in relatively short amounts of time (e.g., about 1 hour or less).

As the COVID-19 pandemic has highlighted, there is a critical need for rapid, accurate systems and methods for diagnosing diseases—particularly infectious diseases. In the absence of diagnostic testing, asymptomatic infected individuals may unknowingly spread the disease to others, and symptomatic infected individuals may not receive appropriate treatment. With testing, however, infected individuals may take appropriate precautions (e.g., self-quarantine) to reduce the risk of infecting others and may receive targeted treatment. In the case of non-infectious diseases, such as cancer, early detection and accurate diagnoses may be critical to successful intervention.

While diagnostic tests for various diseases, including COVID-19, are known, such tests often require specialized knowledge of laboratory techniques and/or expensive laboratory equipment. For example, polymerase chain reaction (PCR) tests generally require skilled technicians and expensive, bulky thermocyclers. PCR tests and other known diagnostic tests with high levels of accuracy often take hours, or even days, to return results, and more rapid tests generally have low levels of accuracy. Thus, there is a need for diagnostic tests that are both rapid and highly accurate. Additionally, many rapid diagnostic tests detect antibodies, which generally can only reveal whether a person has previously had a disease, not whether the person has an active infection. In contrast, nucleic acid tests (i.e., tests that detect one or more target nucleic acid sequences) may indicate that a person has an active infection.

The diagnostic tests, systems, and methods described herein are highly sensitive and accurate and may be safely and easily operated or conducted by untrained individuals. As a result, the diagnostic tests, systems, and methods may be useful in a wide variety of contexts. For example, in some cases, the diagnostic tests and systems may be available over the counter for use by consumers. In such cases, untrained consumers may be able to self-administer the diagnostic test (or administer the test to friends and family members) in their own homes (or any other location of their choosing) without the assistance of another person. In some cases, the diagnostic tests, systems, or methods may be operated or performed by employees or volunteers of an organization (e.g., a school, a medical office, a business). For example, a school (e.g., an elementary school, a high school, a university) may test its students, teachers, and/or administrators, a medical office (e.g., a doctor's office, a dentist's office) may test its patients, or a business may test its employees for a particular disease. In each case, the diagnostic tests, systems, or methods may be operated or performed by the test subjects (e.g., students, teachers, patients, employees) or by designated individuals (e.g., a school nurse, a teacher, a school administrator, a receptionist). Point-of-care administration is also contemplated herein, where the diagnostic tests, systems, or methods are administered by a trained medical professional in a point-of-care setting. Certain embodiments additionally contemplate a downloadable software component or software ecosystem, which may assist with test result readout and data aggregation.

Diagnostic tests and systems provided herein can include the components needed to detect one or more target nucleic acid sequences (e.g., from one or more pathogens of interest). In some embodiments, each component of a diagnostic test or system described herein is relatively small. Thus, unlike diagnostic systems that require bulky and expensive laboratory equipment (e.g., thermocyclers for PCR tests), diagnostic tests and systems described herein may be easily transported and/or easily stored in homes and businesses. Since expensive laboratory equipment can be avoided, the diagnostic tests, systems, and methods of the present invention may be more cost effective than conventional diagnostic tests.

The diagnostic methods provided herein can include performing one or more tests for target nucleic acid sequences, including testing for the presence or absence of one or more target nucleic acid sequences of one or more pathogens of interest. The diagnostic tests, systems, and methods described herein may be safely and easily operated or conducted by untrained individuals. Unlike conventional diagnostic tests, some embodiments described herein may not require knowledge of even basic laboratory techniques.

It should be appreciated that while some examples of the rapid diagnostic tests, systems, and methods provided herein are discussed in the context of specific pathogens or diseases (e.g., SARS-CoV-2), the techniques are not so limited and can be used with any pathogen or disease in which nucleic acid molecules characteristic to or indicative of such pathogen or disease may be detected. Therefore, the examples provided herein of the various embodiments are intended for exemplary purposes only.

### Overview of Diagnostic Systems & Methods

In some embodiments, a diagnostic system comprises one or more sample-collecting components (e.g., swabs) for collecting a sample from a subject (e.g., a human subject, an animal subject). The diagnostic system may, in some cases, further comprise one or more reagents (e.g., lysis reagents, nucleic acid amplification reagents). In certain embodiments, the one or more reagents comprise isothermal nucleic acid amplification reagents (e.g., reagents for loop-mediated isothermal amplification (LAMP), recombinase polymerase amplification (RPA), nicking enzyme amplification reaction (NEAR), or other isothermal amplification methods). Each of the one or more reagents may be in solid form (e.g., lyophilized, dried, crystallized, air jetted) or liquid form (e.g., in solution). In some embodiments, the diagnostic system comprises one or more reaction tubes, droppers, cartridges, and/or blister packs comprising the one or more reagents. In some embodiments, the diagnostic system further comprises a readout device comprising a detection component (e.g., a lateral flow assay strip, a colorimetric assay). In certain embodiments, the readout device comprises a chimney configured to receive a reaction tube. In certain embodiments, the readout device comprises a cartridge, a blister pack, or any other suitable housing for a detection component. The diagnostic system may additionally comprise a heater. The heater may be separate from other components of the diagnostic system or may be integrated with one or more components of the diagnostic system (e.g., the readout device).

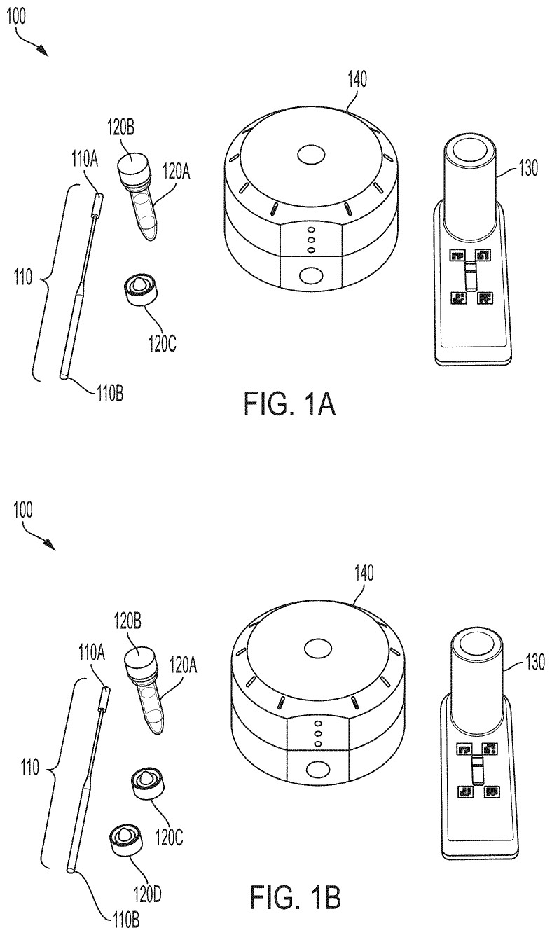

A non-limiting, illustrative embodiment of an exemplary diagnostic system is shown in FIG. 1A. In FIG. 1A, diagnostic system 100 comprises sample-collecting component 110, reaction tube 120, readout device 130, and heater 140. As shown in FIG. 1A, sample-collecting component 110 may comprise swab element 110A and stem element 110B. Reaction tube 120 may comprise tube 120A, first cap 120B, and second cap 120C. First cap 120B and second cap 120C may independently be a screw-top cap or any other type of removable cap, and first cap 120B and second cap 120C may each be configured to fit over an opening of tube 120A. In some cases, first cap 120B and/or second cap 120C are airtight caps (e.g., configured to fit on tube 120A without any gaps). In some embodiments, second cap 120C comprises one or more reagents (e.g., lysis reagents, nucleic acid amplification reagents). The one or more reagents in cap 120C may be in solid form (e.g., lyophilized, dried, crystallized, air jetted) or in liquid form (e.g., in solution). In some cases, one or more reagents are solid and in the form of one or more beads and/or tablets. In certain instances, the one or more beads and/or tablets comprise one or more coatings (e.g., a coating of a time release material). In some embodiments, tube 120A comprises fluidic contents. In certain cases, the fluidic contents of tube 120A comprise one or more buffers (e.g., phosphate-buffered saline (PBS), Tris). In certain cases, the fluidic contents of tube 120A further comprise one or more salts (e.g., magnesium sulfate, ammonium sulfate, potassium chloride, potassium acetate, magnesium acetate tetrahydrate). In certain cases, the fluidic contents of tube 120A further comprise one or more detergents (e.g., Tween 20). The fluidic contents of tube 120A may have any suitable volume.

In operation, a user may collect a sample from a subject (e.g., a human subject, an animal subject) using sample-collecting component 110. In some cases, the subject is the user. In some cases, the subject is another human. In some instances, the user may insert swab element 110A into a nasal or oral cavity of the subject to collect a sample (and, in some cases, may self-collect a sample). After collecting a sample with swab element 110A, first cap 120B may be removed from tube 120A, and swab element 110A may be inserted into the fluidic contents of tube 120A. In some cases, the user may stir swab element 110A in the fluidic contents of tube 120A for a period of time (e.g., at least 10 seconds, at least 15 seconds, at least 20 seconds, at least 30 seconds). In some instances, swab element 110A is removed from tube 120A. In other instances, stem element 110B is broken and removed such that swab element 110A remains in reaction tube 120.

After swab element 110A and/or stem element 110B are removed from tube 120A, a cap may be placed on tube 120A. In some instances, for example, second cap 120C may be placed on tube 120A. In some cases, tube 120A and/or second cap 120C comprise one or more reagents (e.g., lysis reagents, nucleic acid amplification reagents). In some embodiments, one or more reagents may be released from second cap 120C into tube 120A by any suitable mechanism. In some cases, for example, the one or more reagents may be released into tube 120A by securing second cap 120C on tube 120A and inverting (and, in some cases, repeatedly inverting) reaction tube 120. In some cases, second cap 120C comprises a seal (e.g. a foil seal) separating the one or more reagents from the contents of tube 120A, and the seal may be punctured by screwing second cap 120C onto tube 120A, by puncturing the seal with a puncturing tool, or otherwise puncturing the seal. In some cases, the user presses on a button or other portion of second cap 120C and/or twists at least a portion of second cap 120C to release the one or more reagents into tube 120A.

In some embodiments, reaction tube 120 may be inserted into heater 140. Heater 140 may heat reaction tube 120 at one or more temperatures (e.g., at least 37° C., at least 63.5° C., at least 65° C.) for one or more periods of time. In some cases, heating reaction tube 120 according to a first heating protocol (e.g., a first set of temperature(s) and time period(s)) may reduce carryover contamination and/or facilitate lysis of cells within the collected sample. In a particular, non-limiting embodiment, a first heating protocol comprises heating reaction tube 120 at 37° C. for 3-10 minutes (e.g., about 3 minutes). In some cases, heating reaction tube 120 according to a second heating protocol (e.g., a second set of temperature(s) and time period(s)) may facilitate cell lysis and/or amplification of one or more target nucleic acids if present within the sample. In a particular, non-limiting embodiment, a second heating protocol comprises heating reaction tube 120 at 63.5° C. for 5-60 minutes (e.g., about 40 minutes). In some cases, heater 140 may comprise an indicator (e.g., a visual or audio indicator) that a heating protocol is occurring and/or has completed. The indicator may indicate to a user when reaction tube 120 should be removed from heater 140.

Following heating, reaction tube 120 may be inserted into readout device 130. Upon insertion, reaction tube 120 may be punctured by a puncturing component (e.g., a blade, a needle) of readout device 130. In some cases, puncturing reaction tube 120 may cause at least a portion of the fluidic contents of reaction tube 120 to be directed to flow towards (and come into contact with) a lateral flow assay strip of readout device 100. The fluidic contents of reaction tube 120 may flow through the lateral flow assay strip (e.g., via capillary action), and the presence or absence of one or more target nucleic acid sequences may be indicated on a portion of the lateral flow assay strip (e.g., by the formation of one or more visual indicators on the lateral flow assay strip). In some instances, for example, the portion of the lateral flow assay strip may be visible to a user through an opening of readout device 130. In some cases, software (e.g., a mobile application) may be used to read, analyze, and/or report the results (e.g., the one or more visual indicators of the lateral flow assay strip). In some embodiments, readout device 130 comprises one or more markings (e.g., ArUco markers) to facilitate alignment of an electronic device (e.g., a smartphone, a tablet) with readout device 130.

In some embodiments, a diagnostic system comprises a reaction tube comprising at least two caps that each comprise one or more reagents (e.g., lysis reagents, nucleic acid amplification reagents). In certain embodiments, the one or more reagents are in solid form (e.g., lyophilized, dried, crystallized, air jetted). In some cases, the at least two caps may be used to sequentially add reagents to a reaction tube.

FIG. 1B shows an embodiment of diagnostic system 100 comprising reaction tube 120 comprising tube 120A, first cap 120B, second cap 120C, and third cap 120D. In certain cases, second cap 120C and third cap 120D each comprise one or more reagents. In some cases, second cap 120C contains a first set of reagents (e.g., lysis reagents), and third cap 120D comprises a second set of reagents (e.g., nucleic acid amplification reagents). In some cases, the caps may have different colors to indicate that they contain different reagents. For example, second cap 120C may be red, while third cap 120D may be blue. In some cases, the first set of reagents and/or the second set of reagents are in solid form (e.g., lyophilized, dried, crystallized, air jetted). In certain cases, for example, the one or more reagents are in the form of one or more beads and/or tablets. In certain instances, the one or more beads and/or tablets comprise one or more coatings (e.g., a coating of a time release material). In some cases, coatings of different materials and/or thicknesses may delay release of one or more reagents to an appropriate time in the reaction and may facilitate the sequential adding of different reagents. In some instances, the one or more reagents are in liquid form. In addition to reaction tube 120, diagnostic system 100 may comprise sample-collecting component 110, readout device 130, and heater 140.

In operation, a user may collect a sample using sample-collecting component 110 and insert swab element 110A of sample-collecting component 110 into the fluidic contents of tube 120A, as described above. After swab element 110A and/or stem element 110B are removed from tube 120A, a cap may be placed on tube 120A. In some instances, for example, second cap 120C may be placed on tube 120A. In some cases, second cap 120C comprises one or more reagents (e.g., lysis reagents). In some instances, the one or more reagents are in solid form (e.g., lyophilized, dried, crystallized, air jetted). In some cases, for example, the one or more reagents are in the form of one or more beads and/or tablets. In certain instances, the one or more beads and/or tablets comprise one or more coatings (e.g., a coating of a time release material). In some instances, the one or more reagents are in liquid form.

The one or more reagents may be released from second cap 120C into tube 120A by any suitable mechanism. In some cases, the one or more reagents may be released into tube 120A by inverting (and, in some cases, repeatedly inverting) reaction tube 120. In some cases, second cap 120C comprises a seal (e.g. a foil seal) separating the one or more reagents from the contents of tube 120A, and the seal may be punctured by screwing second cap 120C onto tube 120A, by puncturing the seal with a puncturing tool, or otherwise puncturing the seal. In some cases, the user presses on a button or other portion of second cap 120C and/or twists at least a portion of second cap 120C to release the one or more reagents into tube 120A.

In some cases, after the one or more reagents contained in second cap 120C have been added into tube 120A, reaction tube 120 may be heated in heater 140 according to a first heating protocol. In certain embodiments, for example, heating reaction tube 120 according to the first heating protocol may facilitate lysis of cells within the collected sample. In a particular, non-limiting embodiment, a first heating protocol comprises heating reaction tube 220 at 37° C. for 5-10 minutes (e.g., about 3 minutes) and at 65° C. for 5-10 minutes (e.g., about 10 minutes).

After completion of the first heating protocol, second cap 120C may be removed from tube 120A, and third cap 120D may be placed on tube 120A. In some embodiments, third cap 120D comprises one or more reagents (e.g., nucleic acid amplification reagents). In some instances, the one or more reagents are in solid form (e.g., lyophilized, dried, crystallized, air jetted). In some cases, for example, the one or more reagents are in the form of one or more beads and/or tablets. In certain instances, the one or more beads and/or tablets comprise one or more coatings (e.g., a coating of a time release material). In some instances, the one or more reagents are in liquid form.

The one or more reagents may be released from third cap 120D into tube 120A by any suitable mechanism. In some cases, the one or more reagents may be released into tube 120A by inverting (and, in some cases, repeatedly inverting) reaction tube 120. In some cases, third cap 120D comprises a seal (e.g. a foil seal) separating the one or more reagents from the contents of tube 120A, and the seal may be punctured by screwing third cap 120D onto tube 120A, by puncturing the seal with a puncturing tool, or otherwise puncturing the seal. In some cases, the user presses on a button or other portion of third cap 120D and/or twists at least a portion of third cap 120D to release the one or more reagents into tube 120A.

In some cases, after the one or more reagents contained in third cap 120D have been added into tube 120A, reaction tube 120 may be heated in heater 140 according to a second heating protocol. In certain embodiments, for example, heating reaction tube 120 according to the second heating protocol may facilitate amplification of one or more target nucleic acid sequences (if present in the sample). In a particular, non-limiting embodiment, a second heating protocol comprises heating reaction tube 120 at 32° C. for 1-10 minutes (e.g., about 3 minutes), at 65° C. for 10-40 minutes, and at 37° C. for 10-20 minutes (e.g., about 15 minutes).

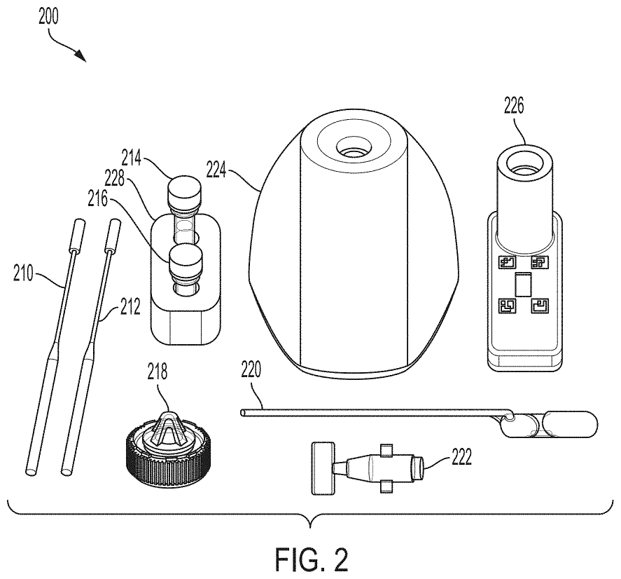

In some embodiments, a diagnostic system comprises one or more additional components (e.g., a pipette, a dropper, one or more sample-collecting elements, one or more reaction tubes). FIG. 2 shows an embodiment of an exemplary diagnostic system 200 comprising first sample-collecting element 210, second sample-collecting element 212, first reaction tube 214, second reaction tube 216, cap 218, pipette 220, dropper 222, heater 224, and readout device 226. Diagnostic system 200 may also comprise tube rack 228 configured to hold first reaction tube 214 and/or second reaction tube 216 upright. In some embodiments, first reaction tube 214 comprises fluidic contents. The fluidic contents may comprise one or more buffers. Cap 218 may be configured such that it fits over any opening of first reaction tube 214 and/or second reaction tube 216. In some cases, cap 218 comprises one or more reagents (e.g., lysis reagents, nucleic acid amplification reagents). In certain embodiments, heater 224 is configured to receive second reaction tube 216. Readout device 226 may also be configured to receive second reaction tube 216. In some embodiments, dropper 222 comprises one or more diluents (e.g., one or more buffers) in liquid form.

In operation, a user may use first sample-collecting element 210A to swab a nasal or oral cavity (e.g., an anterior nares region) for a period of time (e.g., 5 times in each nostril). In some cases, swabbing the nasal or oral cavity with first sample-collecting element 210 may advantageously remove excess material (e.g., nasal matrix) prior to sample collection. The user may then use second sample-collecting element 212 to swab the nasal or oral cavity. The sample-collecting portion of sample-collecting element 212 may then be inserted into first reaction tube 214. In some cases, second sample-collecting element 212 may be stirred in fluidic contents of first reaction tube 214 for an amount of time (e.g., at least 5 seconds, at least 10 seconds, at least 15 seconds, at least 20 seconds, at least 30 seconds). At least a portion of second sample-collecting element 212 may then be removed and discarded. In some cases, the user may transfer an amount of fluid from first reaction tube 214 to second reaction tube 216. For example, the user may use pipette 220 to transfer an amount of fluid from first reaction tube 214 to second reaction tube 216. Cap 218 may then be placed on second reaction tube 216. Second reaction tube 218 may then be inverted and/or shaken to dissolve and mix the reagent bead into the liquid. Following mixing, second reaction tube 216 may be snapped downward to move liquid to the bottom of the reaction tube. Second reaction tube 216 may then be inserted into heater 224. Second reaction tube 216 may then be heated for a first period of time (e.g., about 60 minutes or less, about 55 minutes or less, about 50 minutes or less, about 40 minutes or less, or about 30 minutes or less). Dropper 222 may then be opened, and the contents of dropper 260 may be inserted into the chimney of readout device 226. After the first period of time for heating has elapsed (which may, in some cases, be indicated via one or more visual indicators on heater 224), second reaction tube 216 may be removed from heater 224 and inserted into the chimney of readout device 226. Liquid flow may be initiated (e.g., by one or more motions, such as tapping against a work surface 3 times), and at least a portion of the fluidic contents of second reaction tube 226 may be directed to flow towards a detection component (e.g., lateral flow assay strip) within readout device 226. The results may then be read through an opening in readout device 226.

In some embodiments, each component of the diagnostic system may be shelf stable for a relatively long period of time. In certain embodiments, for example, one or more components (or, in some cases, each component) of the diagnostic system may be stored at room temperature (e.g., 20° C. to 25° C.) for a relatively long period of time (e.g., at least 1 month, at least 3 months, at least 6 months, at least 9 months, at least 1 year, at least 5 years, at least 10 years). In certain embodiments, one or more components (or, in some cases, each component) of the diagnostic system may be stored across a range of temperatures (e.g., 0° C. to 20° C., 0° C. to 37° C., 0° C. to 60° C., 0° C. to 90° C., 20° C. to 37° C., 20° C. to 60° C., 20° C. to 90° C., 37° C. to 60° C., 37° C. to 90° C., 60° C. to 90° C.) for a relatively long period of time (e.g., at least 1 month, at least 3 months, at least 6 months, at least 9 months, at least 1 year, at least 5 years, at least 10 years).

In some embodiments, a diagnostic system described herein is configured to detect one or more target nucleic acids in a sample having a relatively low concentration of the target nucleic acid (e.g., the system has a relatively low limit of detection for the one or more target nucleic acids). In certain embodiments, the diagnostic system is configured to detect a target nucleic acid (e.g., a nucleic acid of SARS-CoV-2, a SARS-CoV-2 variant, an influenza virus, or another pathogen) at a concentration of at least 5 genomic copies per μL, at least 6 genomic copies per μL, at least 7 genomic copies per μL, at least 8 genomic copies per μL, at least 9 genomic copies per μL, at least 10 genomic copies per μL, at least 15 genomic copies per μL, or at least 20 genomic copies per μL. In certain embodiments, the diagnostic system is configured to detect a target nucleic acid at a concentration in a range from 5-6 genomic copies per μL, 5-7 genomic copies per μL, 5-8 genomic copies per μL, 5-9 genomic copies per μL, 5-10 genomic copies per μL, 5-15 genomic copies per μL, 5-20 genomic copies per μL, 8-10 genomic copies per μL, 8-15 genomic copies per μL, 8-20 genomic copies per μL, 10-15 genomic copies per μL, or 10-20 genomic copies per μL.

In some embodiments, the diagnostic system produces invalid results at a relatively low rate. An invalid result may be determined based on characteristics of the diagnostic system. In certain cases, for example, the diagnostic system comprises a readout device comprising a lateral flow strip comprising one or more test lines and one or more control lines (e.g., a flow control line, a sample processing line), and an invalid result may occur when a flow control line is not visible on the lateral flow strip and/or a sample processing control line and a test line are not visible on the lateral flow strip. An invalid rate may refer to the percentage of invalid results (e.g., (number of invalid results/number of total results)×100). In some embodiments, the diagnostic system has an invalid rate of about 30% or less, about 25% or less, about 20% or less, about 15% or less, or about 12% or less. In some embodiments, the diagnostic system has an invalid rate in a range of about 12-15%, 12-20%, 12-25%, 12-30%, 15-20%, 15-25%, 15-30%, 20-25%, 20-30%, or 25-30%.

In some embodiments, the diagnostic system has a relatively high positive percent agreement (PPA) and/or a relatively high negative percent agreement (NPA) with a reference test. In some cases, the diagnostic system may be compared to a reference test by testing a certain number of subjects using both the diagnostic system and the reference test, and positive percent agreement and/or negative percent agreement values may be obtained. Positive percent agreement can be calculated by dividing the number of positive results obtained by the diagnostic system by the number of positive results obtained using the reference test and multiplying by 100. In some embodiments, the diagnostic system has a positive percent agreement with a reference test of at least 90%, at least 91%, at least 92%, at least 93%, at least 94%, at least 95%, at least 96%, at least 97%, at least 98%, at least 99%, or about 100%. In some embodiments, the diagnostic system has a positive percent agreement with a reference test in a range from 90-95%, 90-98%, 90-99%, 90-100%, 95-98%, 95-99%, 95-100%, 98-100%, or 99-100%. Negative percent agreement can be calculated by dividing the number of negative results obtained by the diagnostic system by the number of negative results obtained by the reference test and multiplying by 100. In some embodiments, the diagnostic system has a negative percent agreement with a reference test of at least 90%, at least 91%, at least 92%, at least 93%, at least 94%, at least 95%, at least 96%, at least 97%, at least 98%, at least 99%, or about 100%. In some embodiments, the diagnostic system has a negative percent agreement with a reference test in a range from 90-95%, 90-98%, 90-99%, 90-100%, 95-98%, 95-99%, 95-100%, 98-100%, or 99-100%. In one non-limiting embodiment, the diagnostic system is configured to detect one or more nucleic acids of SARS-CoV-2, and the reference test is the CDC 2019 Novel Coronavirus Real-Time Reverse Transcriptase (RT)-PCR Diagnostic Panel. In another non-limiting embodiment, the diagnostic system is configured to detect one or more nucleic acids of SARS-CoV-2, and the reference test is a Roche Cobas® SARS-CoV-2 test.

Certain aspects are directed to diagnostic methods (e.g., methods of using a diagnostic system described herein). In some embodiments, a diagnostic method comprises collecting a sample from a subject. The sample may be collected according to any method described herein. In some embodiments, collecting the sample comprises collecting a nasal or oral secretion, for example by swabbing a nasal or oral cavity of a subject. In certain embodiments, collecting the sample comprises swabbing an anterior nares region of one or more nostrils of a subject. In some embodiments, the subject performs the collecting step (i.e., the subject self-collects the sample).

In some embodiments, a diagnostic method comprises lysing cells in a collected sample. For example, the diagnostic method may comprise performing a chemical lysis step (e.g., exposing the sample to one or more lysis reagents) and/or performing a thermal lysis step (e.g., heating the sample). In some embodiments, a diagnostic method comprises performing an isothermal nucleic acid amplification reaction configured to amplify one or more target nucleic acids. In certain cases, the nucleic acid amplification reaction may be a LAMP, RPA, NEAR, or other isothermal nucleic acid amplification reaction. In certain embodiments, performing the isothermal nucleic acid amplification reaction comprises contacting a sample with one or more nucleic acid amplification reagents. In some embodiments, performing the isothermal nucleic acid amplification reagent comprises heating the sample for a period of time. The steps for performing each type of nucleic acid amplification reaction are described in further detail herein. In some cases, isothermal nucleic acid amplification may advantageously provide for accurate detection of the presence of small amounts of a target nucleic acid (e.g., 5 genomic copies per μL, 10 genomic copies per μL).

In some embodiments, the diagnostic method comprises detecting the presence or absence of one or more target nucleic acids in the sample. The steps for detecting the one or more target nucleic acids are described in further detail herein. In some embodiments, a diagnostic method comprises analyzing one or more test lines and/or one or more control lines of a lateral flow test strip.

In some embodiments, the total time for performing the diagnostic method is about 100 minutes or less, about 90 minutes or less, about 80 minutes or less, about 75 minutes or less, about 70 minutes or less, about 65 minutes or less, about 60 minutes or less, about 50 minutes or less, 45 minutes or less, about 40 minutes or less, or about 30 minutes or less. In some embodiments, the total time for performing the diagnostic method is in a range from 30 to 40 minutes, 30 to 45 minutes, 30 to 50 minutes, 30 to 60 minutes, 30 to 65 minutes, 30 to 70 minutes, 30 to 75 minutes, 30 to 80 minutes, 30 to 90 minutes, 30 to 100 minutes, 45 to 60 minutes, 45 to 65 minutes, 45 to 70 minutes, 45 to 75 minutes, 45 to 80 minutes, 45 to 90 minutes, 45 to 100 minutes, 60 to 70 minutes, 60 to 75 minutes, 60 to 80 minutes, 60 to 90 minutes, 60 to 100 minutes, 70 to 75 minutes, 70 to 80 minutes, 70 to 90 minutes, 70 to 100 minutes, 75 to 80 minutes, 75 to 90 minutes, 75 to 100 minutes, 80 to 90 minutes, or 80 to 100 minutes.

### Sample Collection

In some embodiments, a diagnostic method comprises collecting a sample from a subject (e.g., a human subject, an animal subject). In some embodiments, a diagnostic system comprises a sample-collecting component configured to collect a sample from a subject (e.g., a human subject, an animal subject). In some embodiments, a diagnostic test is performed on a sample obtained from a subject and/or collected using a sample-collecting component configured to collect a sample from a subject. In some embodiments, the sample comprises a mucus (e.g., nasal secretion), sputum (e.g., a mixture of saliva and mucus), or saliva (e.g., spit) sample or specimen. However, other sample types are envisioned, including, for example, bodily fluids (e.g. blood, serum, plasma, amniotic fluid, urine, cerebrospinal fluid, lymph, tear fluid, feces, or gastric fluid), cell scrapings (e.g., a scraping from the mouth or interior cheek), exhaled breath particles, tissue extracts, culture media (e.g., a liquid in which a cell, such as a pathogen cell, has been grown), environmental samples, agricultural products or other foodstuffs, and their extracts.

The terms “sample” and “specimen” are used interchangeably herein and refer to a quantity of biological material collected from a subject. In some embodiments, the subject collects a sample from themselves (i.e., the sample is “self-collected”). In some embodiments, a separate user collects the sample from the subject. The user may, in some cases, be a health care professional (e.g., a clinician). In other cases, the user may not be medically trained. For example, in some embodiments, a sample is collected from the subject by a second person who is a friend, family member, coworker, or any other person assisting the subject with administration of the rapid diagnostic tests, systems, and methods described herein.

In some embodiments, the sample is a mucus sample. Mucus samples may include, but are not limited to, nasopharyngeal specimens, oropharyngeal specimens, mid-turbinate nasal specimens, and anterior nares specimens. In some embodiments, the sample is a sputum sample or specimen. In some embodiments, the sample is a saliva sample or specimen. Any of these sample or specimen types can be obtained (e.g., collected) using an absorbent material (e.g., a swab or pad). The absorbent material may be any absorbent material suitable for oral or nasal use, such as cotton, filter paper, cellulose-based materials, polyurethane, polyester, and rayon. In some embodiments, the swab is a foam swab. In some embodiments, the swab is a flocked swab or a polyester swab.

### Mucus Samples

Nasopharyngeal Specimens

In some embodiments, the sample is a nasopharyngeal specimen. A nasopharyngeal specimen generally refers to a specimen collected from the upper part of the pharynx, which connects with the nasal cavity above the soft palate. Collection of nasopharyngeal specimens from the surface of the respiratory mucosa may be used for the diagnosis of respiratory infections (e.g., viral respiratory infections) in adults and children. Nasopharyngeal specimens may, in some embodiments, be preferable to other types of specimens because samples obtained from the nasopharynx have been shown to have a higher concentration of viral particles (e.g., a higher viral titer), and thus may provide more accurate diagnostic testing than other relevant sample types with lower viral titers (see, e.g., Callahan, et al. (2020), Nasal-Swab Testing Misses Patients with Low SARS-CoV-2 Viral Loads, medRxiv, preprint available online (PMID: 32587981)). However, collection of a nasopharyngeal specimen is not conducive to self-collection, nor to collection by a second person who is not medically trained.

Non-limiting methods of nasopharyngeal specimen collection are described in the United States Centers for Disease Control and Prevention (CDC) Specimen Collection Guidelines (hereinafter the “CDC Guidelines”), Marty, et al. (2020), How to Obtain a Nasopharyngeal Swab Specimen, New Engl. J. Med., 382:e76, and Cohen, et al. (2020), Optimum Naso-oropharyngeal Swab Procedure for COVID-19: Step-by-Step Preparation and Technical Hints, Comp. Otolaryngology, 130(11): 2564-2567. One non-limiting method for obtaining a nasopharyngeal specimen is described as follows:
Subject blows nose into a tissue to clear excess secretions from the nasal passages.
A swab is inserted along the nasal septum, just above the floor of the nasal passage, to the nasopharynx (e.g., parallel to the palate, not upwards), until resistance is felt. The swab should reach a depth equal to the distance from the nostrils to the outer opening of the ear.
Optionally, the swab is rotated in place several times.
The swab is left in place for several seconds to absorb secretions.
The swab is removed slowly while rotating it.

In some embodiments, the sample is a nasopharyngeal specimen collected by inserting a swab in the nasal passage of a user to the point at which the nasopharynx is contacted and contacting the nasopharynx with the swab for a period of time (e.g., 3-5 seconds). In some embodiments, the swab is rotated one or more times (e.g., 1 time, 2 times, 3 times, 4 times, or 5 times) while still contacted with the nasopharynx. In some embodiments, the swab is subsequently rotated and removed in a continuous motion.

Oropharyngeal Specimens

In some embodiments, the sample is an oropharyngeal specimen. An oropharyngeal specimen generally refers to a specimen collected from the part of the pharynx that lies between the soft palate and the hyoid bone. Collection of oropharyngeal specimens from the surface of the respiratory mucosa may be used for the diagnosis of respiratory infections (e.g., viral respiratory infections) in adults and children. Oropharyngeal specimens may, in some embodiments, be preferable to other types of specimens because obtaining an oropharyngeal specimen is less invasive and results in less user discomfort than certain other sampling methods (e.g., obtaining a nasopharyngeal specimen), yet still yields adequate sample material for a rapid diagnostic test. Additionally, collection of an oropharyngeal specimen is technically less complex and is conducive to self-collection, or collection by a user who is not medically trained.

Non-limiting methods of oropharyngeal specimen collection are described in the CDC Guidelines and in Cohen, et al. (2020), Optimum Naso-oropharyngeal Swab Procedure for COVID-19: Step-by-Step Preparation and Technical Hints, Comp. Otolaryngology, 130(11): 2564-2567. One non-limiting method for obtaining an oropharyngeal specimen is described as follows:
A swab is inserted into the posterior pharynx and tonsillar areas of a subject.
The swab is rubbed over both tonsillar pillars and posterior oropharynx, while avoiding touching the tongue, teeth, and gums.

In some embodiments, the sample is an oropharyngeal specimen collected by inserting a swab into the posterior pharynx and tonsillar areas of a subject to the point at which the oropharynx is contacted and rubbing both tonsillar pillars and the oropharynx with the swab for a period of time (e.g., 3-5 seconds). In some embodiments, the swab does not contact the tongue, teeth, or gums of the subject.

Nasal Mid-Turbinate Specimens

In some embodiments, the sample is a nasal mid-turbinate specimen. A nasal mid-turbinate specimen generally refers to a specimen collected from the middle turbinates of the nose, which are located along the sides of the nasal cavities, are made of bone, and are covered by soft tissue known as mucosa. Collection of nasal mid-turbinate specimens from the surface of the respiratory mucosa may be used for the diagnosis of respiratory infections (e.g., viral respiratory infections) in adults and children. Nasal mid-turbinate specimens may, in some embodiments, be preferable to other types of specimens because obtaining an nasal mid-turbinate specimen is less invasive and results in less user discomfort than certain other sampling methods (e.g., obtaining a nasopharyngeal specimen), yet still yields adequate sample material for a rapid diagnostic test. Additionally, collection of a nasal mid-turbinate specimen is technically less complex and is conducive to self-collection or collection by a user who is not medically trained.

Non-limiting methods of nasal mid-turbinate specimen collection are described in the CDC in its Nasal Mid-Turbinate (NMT) Specimen Collection Steps infographic. One non-limiting method for obtaining a nasal mid-turbinate specimen is described as follows:
While gently rotating a swab, the swab is inserted less than one inch (about 2 cm) into the nostril of the subject parallel to the palate until resistance is met at the turbinates.
The swab is rotated several times against nasal wall.
The swab is removed, and, optionally, the swab is inserted into the other nostril of the user and the process is repeated.

In some embodiments, the sample is a nasal mid-turbinate specimen collected by inserting a swab into one or both nostrils of a subject, while simultaneously rotating said swab, to the point at which the turbinates are contacted. In some embodiments, the swab is rotated one or more times (e.g., 3-10 times) against the nasal wall of one or more both nostrils prior to removal of the swab. In some embodiments, the swab is rotated at least 3 times, at least 4 times, at least 5 times, at least 6 times, at least 7 times, at least 8 times, at least 9 times, or at least 10 times.

Anterior Nares Specimens

In some embodiments, the sample is an anterior nares specimen. The anterior nares (e.g., nostrils) generally refer to the external portions of the nose which open into the nasal cavity and allow the inhalation and exhalation of air. An anterior nares specimen may also be referred to as a nasal specimen. Nasal specimens may, in some embodiments, be preferable to other types of specimens because obtaining a nasal specimen is less invasive and results in less user discomfort than certain other sampling methods (e.g., obtaining a nasopharyngeal specimen), yet still yields adequate sample material for a rapid diagnostic test (see, e.g., Péré, et al. (2020) Nasal Swab Sampling for SARS-CoV-2: a Convenient Alternative in Times of Nasopharyngeal Swab Shortage, J. Clin. Microbiol., 58(6):e00721-20). Additionally, collection of a nasal specimen is technically less complex and is highly conducive to self-collection or collection by a user who is not medically trained.

Non-limiting methods of nasal specimen collection are described in the CDC Guidelines. One non-limiting method for obtaining a nasal (anterior nares) specimen is as follows:
A swab is inserted at least 1 cm (0.5 inch) inside the nostril (naris) of the subject, and the nasal membrane is sampled by rotating the swab and then leaving the swab in place for 10 to 15 seconds.
The swab is removed, and, optionally, the swab is inserted into the other nostril of the user and the process is repeated.

In some embodiments, the nostrils are cleared of excess nasal material prior to sample collection by inserting a swab into one or more nostrils of a subject and swabbing the one or more nostrils for a period of time (e.g., 10-15 seconds). In some embodiments, one or more nostrils are swabbed for at least 10 seconds, at least 11 seconds, at least 12 seconds, at least 13 seconds, at least 14 seconds, or at least 15 seconds each. In some embodiments, the nostrils are cleared of excess nasal material prior to sample collection by inserting a swab into one or more nostrils of a subject and swabbing each of the one or more nostrils one or more times (e.g., 1-3 times, 1-5 times, 1-10 times, 3-10 times, 5-10 times). In some embodiments, one or more nostrils are swabbed at least 1 time, at least 2 times, 3 times, at least 4 times, at least 5 times, at least 6 times, at least 7 times, at least 8 times, at least 9 times, or at least 10 times each. In certain instances, the nostrils are cleared of excess nasal material prior to sample collection by inserting a swab into each nostril of a user and swabbing both nostrils 5 times each. In embodiments that include a step of clearing excess nasal material prior to sample collection, a first swab may be used for the step of clearing excess nasal material, and a second, separate swab may be used for the step of collecting a sample.

In some embodiments, the sample is an anterior nares specimen collected by inserting a swab into one or more nostrils of the subject for a period of time. In some embodiments, the period of time is at least 3 seconds, at least 5 seconds, at least 10 seconds, at least 15 seconds, at least 20 seconds, or at least 30 seconds. In some embodiments, the period of time is 30 seconds or less, 20 seconds or less, 15 seconds or less, 10 seconds or less, 5 seconds or less, or 3 seconds or less. In some embodiments, the period of time is in a range from 3 seconds to 5 seconds, 3 seconds to 10 seconds, 3 seconds to 15 seconds, 3 seconds to 20 seconds, 3 seconds to 30 seconds, 5 seconds to 10 seconds, 5 seconds to 15 seconds, 5 seconds to 20 seconds, 5 seconds to 30 seconds, 10 seconds to 20 seconds, or 10 seconds to 30 seconds. In some embodiments, the sample is an anterior nares specimen collected by inserting a swab into one or more nostrils of a subject and swabbing each of the one or more nostrils one or more times (e.g., 1-3 times, 1-5 times, 1-10 times, 3-5 times, 3-10 times, 5-10 times). In some embodiments, the anterior nares specimen is collected by inserting a swab into one or more nostrils and swabbing each of the one or more nostrils at least 1 time, at least 2 times at least 3 times, at least 4 times, at least 5 times, at least 6 times, at least 7 times, at least 8 times, at least 9 times, or at least 10 times. In certain embodiments, the sample is an anterior nares specimen collected by inserting a swab into each nostril of a user and swabbing both nostrils 5 times each.

### Sputum Samples

In some embodiments, the sample is a sputum (e.g., phlegm) specimen. Sputum, also known as phlegm, generally refers to mucus that is thicker than normal due to illness or irritation and that may be coughed up from the respiratory tract of a user. Sputum specimens may, in some embodiments, be preferable to other types of specimens because obtaining a sputum specimen is less invasive and results in less user discomfort than certain other sampling methods (e.g., obtaining a nasopharyngeal specimen) and requires no swab or swab-like apparatus, yet still yields adequate sample material for a rapid diagnostic test. Additionally, collection of a sputum specimen is not technically complex and is highly conducive to self-collection.

Sputum specimens may also, in some embodiments, be preferable to other types of specimens because (1) the rate of SARS-CoV-2 detection has been shown to be significantly higher in sputum specimens than either oropharyngeal or nasopharyngeal specimens (see, e.g., Mohammadi, et al. (2020), SARS-CoV-2 detection in different respiratory sites: A systematic review and meta-analysis, EBio Medicine, 59:102903), (2) the viral concentration in sputum samples has been shown to equate to or exceed that present in other relevant sample types (e.g., nasopharyngeal and oropharyngeal specimens; see, e.g., Wölfel, et al. (2020), Virological assessment of hospitalized patients with COVID-2019, Nature, 581: 465-469), and (3) the shedding of viral RNA from sputum has been shown to outlast the end of symptoms in COVID-19 patients (see, e.g., Wölfel, et al. (2020), Nature, 581: 465-469).

Non-limiting methods of sputum specimen collection are described in the CDC Guidelines. One non-limiting method for obtaining a sputum specimen is as follows:
The mouth of the subject is rinsed with water, and the water is spit out by the subject.
The subject inhales and coughs deeply until sputum is released into the mouth of the subject.
Once sputum is in the mouth of the subject, the sputum specimen is expectorated into a sterile sample tube or sterile sample container.

In some embodiments, if the sample is self-collected by the subject, the sample is a sputum specimen collected by rinsing the mouth with water, coughing sputum into the mouth, and depositing the sputum specimen into a sterile sample tube or sterile sample container. In some embodiments, if the sample is collected by a separate person, the sample is a sputum specimen collected by instructing the subject to rinse their mouth with water, instructing the subject to cough sputum into their mouth, and instructing the subject to expectorate the sputum specimen into a sterile sample tube or sterile sample container. Sample tubes and sample containers are described elsewhere herein.

In some embodiments, the procedure for obtaining the sputum specimen may need to be repeated multiple times in order to obtain a sample of adequate volume for rapid diagnostic testing. In some embodiments, an adequate volume for rapid diagnostic testing is 1-10 mL or more. Accordingly, in some embodiments, a sputum specimen has a volume of at least 1 mL, at least 1.5 mL, at least 2 mL, at least 2.5 mL, at least 3 mL, at least 3.5 mL, at least 4 mL, at least 4.5 mL, at least 5 mL, at least 5.5 mL, at least 6 mL, at least 6.5 mL, at least 7 mL, at least 7.5 mL, at least 8 mL, at least 8.5 mL, at least 9 mL, at least 9.5 mL, or at least 10 mL.

### Saliva Samples

In some embodiments, the sample is a saliva specimen. Saliva generally refers to watery liquid secreted into the mouth by glands, providing lubrication for chewing and swallowing and aiding digestion. Saliva specimens may, in some embodiments, be preferable to other types of specimens because obtaining a saliva specimen is less invasive and results in less user discomfort than certain other sampling methods (e.g., obtaining a nasopharyngeal specimen), and requires no swab or swab-like apparatus, yet still yields adequate sample material for a rapid diagnostic test. Additionally, collection of a saliva specimen is not technically complex and is highly conducive to self-collection.

In some embodiments, the sample is a saliva specimen collected by collecting saliva in a sterile sample tube or sterile sample container. Sample tubes and sample containers are described elsewhere herein. The volume of saliva in the specimen may be 1-5 mL or more. In some embodiments, the saliva specimen has a volume at least 1 mL, at least 1.5 mL, at least 2 mL, at least 2.5 mL, at least 3 mL, at least 3.5 mL, at least 4 mL, at least 4.5 mL, or at least 5 mL. In some embodiments, the saliva specimen has a volume in a range from 1 mL to 2 mL, 1 mL to 3 mL, 1 mL to 4 mL, 1 mL to 5 mL, 2 mL to 4 mL, 2 mL to 5 mL, or 3 mL to 5 mL.

### Sample Characteristics

The concentration of target nucleic acid molecules within a sample may vary depending on the nature of the pathogen, the stage of infection at which the sample is collected, the severity of infection, the type of sample, and the general health condition of the subject, among other factors. For example, saliva has been found to have a mean concentration of SARS-Cov-2 RNA of 5 fM (Kai-Wang To, et al., 2020). Sputum has been found to have a mean concentration of SARS-Cov-2 RNA of 7.52×10⁵ copies/mL (Pan, et al. (2020), Viral load of SARS-CoV-2 in clinical samples, Lancet Infect Dis., 20(4): 411-412), and, in another report, 7.00×10⁶ copies per mL (with a maximum of 2.35×10⁹ copies per mL; Wölfel, et al. (2020), Nature, 581: 465-469). Each of these concentrations is detectable by any one of the diagnostic tests, systems, and/or methods described herein.

In some embodiments, the concentration of a target nucleic acid molecule (e.g., SARS-CoV-2 RNA) in the sample is at least 5 aM, at least 10 aM, at least 15 aM, at least 20 aM, at least 25 aM, at least 30 aM, at least 35 aM, at least 40 aM, at least 50 aM, at least 75 aM, at least 100 aM, at least 150 aM, at least 200 aM, at least 300 aM, at least 400 aM, at least 500 aM, at least 600 aM, at least 700 aM, at least 800 aM, at least 900 aM, at least 1 fM, at least 5 fM, at least 10 fM, at least 15 fM, at least 20 fM, at least 25 fM, at least 30 fM, at least 35 fM, at least 40 fM, at least 50 fM, at least 75 fM, at least 100 fM, at least 150 fM, at least 200 fM, at least 300 fM, at least 400 fM, at least 500 fM, at least 600 fM, at least 700 fM, at least 800 fM, at least 900 fM, at least 1 pM, at least 5 pM, or at least 10 pM. In some embodiments, the concentration of a target nucleic acid molecule (e.g., SARS-CoV-2 RNA) in the sample is 10 pM or less, 5 pM or less, 1 pM or less, 500 fM or less, 100 fM or less, 50 fM or less, 10 fM or less, 1 fM or less, 500 aM or less, 100 aM or less, 50 aM or less 10 aM or less, or 5 aM or less.

In some embodiments, the concentration of a target nucleic acid molecule (e.g., SARS-CoV-2 RNA) in the sample is in a range from 5 aM to 50 aM, 5 aM to 100 aM, 5 aM to 500 aM, 5 aM to 1 fM, 5 aM to 10 fM, 5 aM to 50 fM, 5 aM to 100 fM, 5 aM to 500 fM, 5 aM to 1 pM, 5 aM to 10 pM, 10 aM to 50 aM, 10 aM to 100 aM, 10 aM to 500 aM, 10 aM to 1 fM, 10 aM to 10 fM, 10 aM to 50 fM, 10 aM to 100 fM, 10 aM to 500 fM, 10 aM to 1 pM, 10 aM to 10 pM, 100 aM to 500 aM, 100 aM to 1 fM, 100 aM to 10 fM, 100 aM to 50 fM, 100 aM to 100 fM, 100 aM to 500 fM, 100 aM to 1 pM, 100 aM to 10 pM, 1 fM to 10 fM, 1 fM to 50 fM, 1 fM to 100 fM, 1 fM to 500 fM, 1 fM to 1 pM, 1 fM to 10 pM, 5 fM to 10 fM, 5 fM to 50 fM, 5 fM to 100 fM, 5 fM to 500 fM, 5 fM to 1 pM, 5 fM to 10 pM, 10 fM to 100 fM, 10 fM to 500 fM, 10 fM to 1 pM, 10 fM to 10 pM, 100 fM to 500 fM, 100 fM to 1 pM, 100 fM to 10 pM, or 1 pM to 10 pM.

### Target Nucleic Acid Sequences

The rapid diagnostic tests, systems, and methods described herein are, in some embodiments, intended to detect the presence of one or more target nucleic acid sequences in human or animal subjects (e.g., subjects having or suspected of having a pathogenic infection). In certain embodiments, a test sample is obtained from a subject who has been infected by, or is suspected of having been infected by, one or more pathogens that are detectable using the rapid diagnostic test.

As used herein, the terms “subject” and “patient” are used interchangeably to refer to the human or animal subject to whom the rapid diagnostic test of the disclosure is being applied. Because the rapid diagnostic test is, in some embodiments, contemplated for self-use, where the subject self-collects the test sample and directly practices the test methods, the subject may in some cases also be referred to as a “user” (e.g., a user of the rapid diagnostic test).

A “pathogen” is any organism capable of causing disease, and may include viruses, bacterium, protozoans, prions, viroids, parasite, and/or fungi. Any pathogen may be detected using the rapid diagnostic tests, methods, or systems of the present disclosure.

In some embodiments, the one or more pathogens comprise a viral pathogen. Non-limiting examples of viral pathogens include coronaviruses, influenza viruses, rhinoviruses, parainfluenza viruses (e.g., parainfluenza 1-4), enteroviruses, adenoviruses, respiratory syncytial viruses, and metapneumoviruses. In certain embodiments, the viral pathogen is SARS-CoV-2. In some embodiments, the viral pathogen is a variant of SARS-CoV-2. In certain instances, the variant of SARS-CoV-2 is SARS-CoV-2 D614G, a SARS-CoV-2 variant of B.1.1.7 lineage (e.g., 20B/501Y.V1 Variant of Concern (VOC) 202012/01), or a SARS-CoV-2 variant of B.1.351 lineage (e.g., 20C/501Y.V2). In certain embodiments, the viral pathogen is an influenza virus. The influenza virus may be an influenza A virus (e.g., H1N1, H3N2) or an influenza B virus.

Other viral pathogens include, but are not limited to, adenovirus; Herpes simplex, type 1; Herpes simplex, type 2; encephalitis virus; papillomavirus (e.g., human papillomavirus); Varicella zoster virus; Epstein-Barr virus; human cytomegalovirus; human herpesvirus, type 8; BK virus; JC virus; smallpox; polio virus; hepatitis A virus; hepatitis B virus; hepatitis C virus; hepatitis D virus; hepatitis E virus; human immunodeficiency virus (HIV); human bocavirus; parvovirus B19; human astrovirus; Norwalk virus; coxsackievirus; rhinovirus; Severe acute respiratory syndrome (SARS) virus; yellow fever virus; dengue virus; West Nile virus; Guanarito virus; Junin virus; Lassa virus; Machupo virus; Sabiá virus; Crimean-Congo hemorrhagic fever virus; Ebola virus; Marburg virus; measles virus; mumps virus; rubella virus; Hendra virus; Nipah virus; Rabies virus; rotavirus; orbivirus; Coltivirus; Hantavirus; Middle East Respiratory Coronavirus; Zika virus; norovirus; Chikungunya virus; and Banna virus.

In some embodiments, a viral pathogen comprises a Coronavirinae pathogen. In some embodiments, the Coronavirinae pathogen comprises an Alphacoronavirus, Betacoronavirus, Gammacoronavirus, Deltacoronavirus, Human coronavirus 229E, Human coronavirus NL63, Human coronavirus OC43, Human coronavirus HKU1, Middle East Respiratory Syndrome coronavirus (e.g., MERS-CoV), Severe acute respiratory coronavirus (e.g., SARS-CoV), or Severe acute respiratory syndrome coronavirus 2 (e.g., SARS-CoV-2) pathogen. In some embodiments, the Coronavirinae pathogen causes a pathogenic infection (e.g., a viral disease) in a subject. In some embodiments, the pathogenic infection is a coronavirus disease. In some embodiments, the coronavirus disease is Coronavirus disease 2019 (e.g., COVID-19). In some embodiments, the coronavirus disease is a variant of COVID-19. In some embodiments, the coronavirus disease is Middle East Respiratory Syndrome (MERS). In some embodiments, the coronavirus disease is Severe acute respiratory syndrome (SARS). In some embodiments, the coronavirus disease is Human coronavirus OC43 (HCoV-OC43). In some embodiments, the coronavirus disease is Human coronavirus HKU1 (HCoV-HKU1). In some embodiments, the coronavirus disease is Human coronavirus 229E (HCoV-229E). In some embodiments, the coronavirus disease is Human coronavirus NL63 (HCoV-NL63).

In some embodiments, a viral pathogen comprises an Orthomyxoviridae pathogen. In some embodiments, the Orthomyxoviridae pathogen comprises an Alphainfluenzavirus, Betainfluenzavirus, Deltainfluenzavirus, or Gammainfluenzavirus pathogen. In some embodiments, the Alphainfluenzavirus pathogen comprises an Influenza virus A pathogen. In some embodiments, the Betainfluenzavirus pathogen comprises an Influenza virus B pathogen. In some embodiments, the Gammainfluenzavirus pathogen comprises an Influenza virus C pathogen. In some embodiments, the Orthomyxoviridae pathogen causes a pathogenic infection (e.g., a viral disease) in a subject. In some embodiments, the pathogenic infection is an influenza virus disease. In some embodiments, the influenza virus disease is Influenza A. In some embodiments, the Influenza A virus is of the subtype H1N1, H2N2, H3N2, H5N1, H7N7, H1N2, H9N2, H7N2, H7N3, or H10N7. In some embodiments, the influenza virus disease is Influenza B. In some embodiments, the Influenza B virus is of the lineage Victoria or Yamagata. In some embodiments, the influenza virus disease is Influenza C.

In some embodiments, the one or more pathogens comprise a bacterial pathogen. Non-limiting examples of bacterial pathogens include Gram-positive bacteria and Gram-negative bacteria. Bacterial pathogens include, but are not limited to, Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli O157:H7, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Preteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, and Yersinia pestis.

In some embodiments, the one or more pathogens comprise a fungal pathogen. Non-limiting examples of fungal pathogens include, but are not limited to, Ascomycota (e.g., Fusarium oxysporum, Pneumocystis jirovecii, Aspergillus spp., Coccidioides immitis/posadasii, Candida albicans), Basidiomycota (e.g., Filobasidiella neoformans, Trichosporon), Microsporidia (e.g., Encephalitozoon cuniculi, Enterocytozoon bieneusi), and Mucoromycotina (e.g., Mucor circinelloides, Rhizopus oryzae, Lichtheimia corymbifera).

In some embodiments, the one or more pathogens comprise a protozoan pathogen. Non-limiting examples of protozoan pathogens include, but are not limited to, Entamoeba histolytica, Giardia lambda, Trichomonas vaginalis, Trypanosoma brucei, T. cruzi, Leishmania donovani, Balantidium coli, Toxoplasma gondii, Plasmodium spp., and Babesia microti.

In some embodiments, the one or more pathogens comprise a parasitic pathogen. Non-limiting examples of parasitic pathogens include, but are not limited to, Acanthamoeba, Anisakis, Ascaris lumbricoides, botfly, Balantidium coli, bedbug, Cestoda, chiggers, Cochliomyia hominivorax, Entamoeba histolytica, Fasciola hepatica, Giardia lamblia, hookworm, Leishmania, Linguatula serrata, liver fluke, Loa loa, Paragonimus, pinworm, Plasmodium falciparum, Schistosoma, Strongyloides stercoralis, mite, tapeworm, Toxoplasma gondii, Trypanosoma, whipworm, and Wuchereria bancrofti.

In some embodiments, the diagnostic tests, systems, and methods are configured to detect a target nucleic acid sequence of an animal pathogen. As will be understood, an animal pathogen may be considered a human pathogen in certain instances, for example in cases where a pathogen originating in a non-human animal infects a human. Examples of animal pathogens include, but are not limited to, bovine rhinotracheitis virus, bovine herpesvirus, distemper, parainfluenza, canine adenovirus, rhinotracheitis virus, calicivirus, canine parvovirus, Borrelia burgdorferi (Lyme disease), Bordetella bronchiseptica (kennel cough), canine parainfluenza, leptospirosis, feline immunodeficiency virus, feline leukemia virus, Dirofilaria immitis (heartworm), feline herpesvirus, Chlamydia infections, Bordetella infections, equine influenza, rhinopneumonitis (equine herpesevirus), equine encephalomyelitis, West Nile virus (equine), Streptococcus equi, tetanus (Clostridium tetani), equine protozoal myeloencephalitis, bovine respiratory disease complex, clostridial disease, bovine respiratory syncytial virus, bovine viral diarrhea, Haemophilus somnus, Pasteurella haemolytica, and Pastuerella multocida.

In some cases, a subject may be infected with a single type of pathogen or with multiple types of pathogens simultaneously. A “pathogenic infection” may encompass any of a viral infection, a bacterial infection, protozoan infection, prion disease, viroid infection, parasitic infection, or fungal infection. Any pathogenic infection may be detected using the rapid diagnostic tests, systems, and methods of the present disclosure.

In some embodiments, a pathogenic infection comprises any one of: African sleeping sickness, Amebiasis, Ascariasis, Bronchitis, Candidiasis, Chickenpox, Cholera, Coronavirus, Human coronavirus OC43 (HCoV-OC43), Human coronavirus HKU1 (HCoV-HKU1), Human coronavirus 229E (HCoV-229E), Human coronavirus NL63 (HCoV-NL63), Middle East Respiratory Syndrome (MERS), Severe acute respiratory syndrome (SARS), Coronavirus disease 2019 (COVID-19), Cryptosporidiosis, Dengue fever, Diphtheria, Elephantiasis, Gastric ulcers, Giardiasis, Gonorrhea, Hepatitis A, Hepatitis B, Hepatitis C, Herpes simplex 1, Herpes simplex 2, Hookworm, Influenza, Influenza A, Influenza A(H1N1), Influenza A(H2N2), Influenza A(H3N2), Influenza A(H5N1), Influenza A(H7N7), Influenza A(H1N2), Influenza A(H9N2), Influenza A(H7N2), Influenza A(H7N3), Influenza A(H10N7), Influenza B, Influenza B(Victoria), Influenza B(Yamagata), Influenza C, Leprosy, Malaria, Measles, Meningitis, Mononucleosis, Mumps, Pertussis, Pneumonia, Poliomyelitis, Ringworm, Riverblindness, Rubella, Schistosomiasis, Smallpox, Strep throat, Trachoma, Trichuriasis, Tuberculosis, and Typhoid fever.

In some embodiments, the rapid diagnostic tests, systems, and methods of the present disclosure are applied to a subject who is suspected of having a pathogenic infection or disease, but who has not yet been diagnosed as having such an infection or disease. A subject may be “suspected of having” a pathogenic infection or disease when the subject exhibits one or more signs or symptoms of such an infection or disease. Such signs or symptoms are well known in the art and may vary, depending on the nature of the pathogen and the subject. Signs and symptoms of disease may generally include any one or more of the following: fever, chills, cough (e.g., dry cough), generalized fatigue, sore throat, runny nose, nasal congestion, muscle aches, difficulty breathing (shortness of breath), congestion, runny nose, headaches, nausea, vomiting, diarrhea, loss of smell and/or taste, skin lesions (e.g., pox), or loss of appetite. Other signs or symptoms of disease are specifically contemplated herein. As a non-limiting example, symptoms of coronaviruses (e.g., COVID-19) may include, but are not limited to, fever, cough (e.g., dry cough), generalized fatigue, sore throat, runny nose, nasal congestion, muscle aches, loss of smell and/or taste, and difficulty breathing (shortness of breath). As a non-limiting example, symptoms of influenza may include, but are not limited to, fever, chills, muscle aches, cough, sore throat, runny nose, nasal congestion, and generalized fatigue.

A subject may also be “suspected of having” a pathogenic infection or disease despite exhibiting no signs or symptoms of such an infection or disease (e.g., the subject is asymptomatic). Pathogenic infections can be highly transmissible. In some embodiments, an asymptomatic subject is suspected of having a pathogenic infection or disease due to known contact with an individual having or suspected of having a pathogenic infection or disease (e.g., an individual who tested positive as having a pathogenic infection or disease). In some embodiments, an asymptomatic subject is suspected of having a pathogenic infection or disease due to known contact with an individual having or suspected of having a pathogenic infection or disease within the preceding two-week (e.g., 14 day) time period. In some embodiments, an asymptomatic subject is suspected of having a pathogenic infection or disease due to known contact with an individual who tested positive as having a pathogenic infection or disease within the preceding two-week (e.g., 14 day) time period.

In some embodiments, the diagnostic tests, systems, and methods are configured to detect a target nucleic acid sequence of a cancer cell. Cancer cells have unique mutations found in tumor cells and absent in normal cells. For example, the diagnostic tests, systems, and methods may be configured to detect a target nucleic acid sequence encoding a cancer neoantigen, a tumor-associated antigen (TAA), and/or a tumor-specific antigen (TSA). Examples of TAAs include, but are not limited to, MelanA (MART-I), gplOO (Pmel 17), tyrosinase, TRP-I, TRP-2, MAGE-I, MAGE-3, BAGE, GAGE-I, GAGE-2, pl5(58), CEA, RAGE, NY-ESO (LAGE), SCP-I, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23Hl, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68VKP1, CO-029, FGF-5, G250, Ga733 (EpCAM), HTgp-175, M344, MA-50, MG7-Ag, MOV18, NB/70K, NY-CO-I, RCAS1, SDCCAG16, TA-90 (Mac-2 binding protein\cyclophilin C-associated protein), TAAL6, TAG72, TLP, and TPS5. Neoantigens, in some embodiments, arise from tumor proteins (e.g., tumor-associated antigens and/or tumor-specific antigens). In some embodiments, the neoantigen comprises a polypeptide comprising an amino acid sequence that is identical to a sequence of amino acids within a tumor antigen or oncoprotein (e.g., Her2, E7, tyrosinase-related protein 2 (Trp2), Myc, Ras, or vascular endothelial growth factor (VEGF)). In some embodiments, the amino acid sequence comprises at least 10, at least 15, at least 20, at least 25, at least 30, at least 35, at is least 40, at least 45, at least 50, at least 75, at least 100, at least 150, at least 200, or at least 250 amino acids. In some embodiments, the amino acid sequence comprises 10-250, 50-250, 100-250, or 50-150 amino acids.

In some embodiments, the diagnostic tests, systems, and methods are configured to examine a subject's predisposition to certain types of cancer based on specific genetic mutations. As an example, mutations in BRCA1 and/or BRCA2 may indicate that a subject is at an increased risk of breast cancer, as compared to a subject who does not have mutations in the BRCA1 and/or BRCA2 genes. In some instances, the diagnostic devices, systems, and methods are configured to detect a target nucleic acid sequence comprising a mutation in BRCA1 and/or BRCA2. Other genetic mutations that may be screened according to the diagnostic devices, systems, and methods provided herein include, but are not limited to, BARD1, BRIP1, TP53, PTEN, MSH2, MLH1, MSH6, NF1, PMS1, PMS2, EPCAM, APC, RB1, MEN1, MEN2, and VHL. Further, determining a subject's genetic profile may help guide treatment decisions, as certain cancer drugs are indicated for subjects having specific genetic variants of particular cancers. For example, azathioprine, 6-mercaptopurine, and thioguanine all have dosing guidelines based on a subject's thiopurine methyltransferase (TPMT) genotype (see, e.g., The Pharmacogeneomics Knowledgebase, pharmgkb.org).

In some embodiments, the diagnostic tests, systems, and methods are configured to detect a target nucleic acid sequence associated with a genetic disorder. Non-limiting examples of genetic disorders include hemophilia, sickle cell anemia, α-thalassemia, β-thalassemia, Duchene muscular dystrophy (DMD), Huntington's disease, severe combined immunodeficiency, Marfan syndrome, hemochromatosis, and cystic fibrosis. In some embodiments, the target nucleic acid sequence is a portion of nucleic acid from a genomic locus of at least one of the following genes: CFTR, FMR1, SMN1, ABCB 11, ABCC8, ABCD1, ACAD9, ACADM, ACADVL, ACAT1, AC0X1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASP A, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CUT A, CLN3, CLN5, CLN6, CLN8, CLRN1, CNGB3, COL27A1, COL4A3, COL4A4, COL4A5, COL7A1, CPS1, CPT1A, CPT2, CRB 1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBAI, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAM A3, LAMB3, LAMC2, LCA5, LDLR, LDLRAP1, LHX3, LIFR, LIP A, LOXHD1, LPL, LRPPRC, MAN2B1, MCOLN1, MED 17, MESP2, MFSD8, MKS1, MLC1, MMAA, MMAB, MMACHC, MMADHC, MPI, MPL, MPV17, MTHFR, MTM1, MTRR, MTTP, MUT, MY07A, NAGLU, NAGS, NBN, NDRG1, NDUFAF5, NDUFS6, NEB, NPC1, NPC2, NPHS1, NPHS2, NR2E3, NTRK1, OAT, OP A3, OTC, PAH, PC, PCCA, PCCB, PCDH15, PDHAl, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TORG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

The diagnostic tests, systems, and methods described herein may also be used to test water or food for contaminants (e.g., for the presence of one or more bacterial toxins). Bacterial contamination of food and water can result in foodborne diseases, which contribute to approximately 128,000 hospitalizations and 3000 deaths annually in the United States (CDC, 2016). In some cases, the diagnostic tests, systems, and methods described herein may be used to detect one or more toxins (e.g., bacterial toxins). In particular, bacterial toxins produced by Staphylococcus spp., Bacillus spp., and Clostridium spp. account for the majority of foodborne illnesses. Non-limiting examples of bacterial toxins include toxins produced by Clostridium botulinum, C. perfringens, Staphylococcus aureus, Bacillus cereus, Shiga-toxin-producing Escherichia coli (STEC), and Vibrio parahemolyticus. Exemplary toxins include, but are not limited to, aflatoxin, cholera toxin, diphtheria toxin, Salmonella toxin, Shiga toxin, Clostridium botulinum toxin, endotoxin, and mycotoxin. By testing a potentially contaminated food or water sample using the diagnostic tests, systems, or methods described herein, one can determine whether the sample contains the one or more bacterial toxins. In some embodiments, the diagnostic tests, systems, or methods may be operated or conducted during a food production process to ensure food safety prior to consumption.

In some embodiments, the diagnostic tests, systems, and methods described herein may be used to test samples of soil, building materials (e.g., drywall, ceiling tiles, wall board, fabrics, wall paper, and floor coverings), air filters, environmental swabs, or any other sample. In certain embodiments, the diagnostic devices, systems, and methods may be used to detect one or more toxins, as described above. In certain instances, the diagnostic tests, systems, and methods may be used to analyze ammonia- and methane-oxidizing bacteria, fungi or other biological elements of a soil sample. Such information can be useful, for example, in predicting agricultural yields and in guiding crop planting decisions.

### Sample Processing

As described elsewhere herein, aspects of the invention involve collecting a sample from a subject (e.g., a subject having or suspected of having a pathogenic infection) for use in diagnostic testing. Samples as contemplated herein comprise cells (e.g., pathogenic cells), which comprise nucleic acid molecules (e.g., deoxyribonucleic acid (DNA) molecules, ribonucleic acid (RNA) molecules). Such nucleic acid molecules comprised in the sample may be of host or non-host origin. For example, if a pathogenic infection is present, the nucleic acid molecules comprised in the sample may be of pathogenic (e.g., non-host) origin.

To facilitate rapid and accurate detection of target nucleic acids, the tests, systems, and methods of the present invention encompass methods of nucleic acid amplification whereby target nucleic acid sequences (e.g., target nucleic acids) are amplified. In addition to a step of amplifying the target nucleic acids, methods of nucleic acid amplification may also encompass steps of cell lysis and, in some embodiments, nucleic acid extraction and purification.

### Cell Lysis

In some embodiments, a step of cell lysis is performed to access the intracellular contents (e.g., nucleic acid molecules) of cells within a sample collected from a subject. Cell lysis generally refers to a method in which the outer boundary or cell membrane is broken down or destroyed to release intracellular materials (e.g., DNA, RNA, proteins, organelles, etc.) from a cell. Methods of cell lysis include, for example, chemical, thermal, enzymatic, and/or mechanical treatment of the cells (see, e.g., Barbosa, et al. In Molecular Microbial Diagnostic Methods (eds. Martin D'Agostino & K. Clive Thompson) 135-154 (Academic Press, 2016); Islam, et al. (2017), A Review on Macroscale and Microscale Cell Lysis Methods, Micromachines (Basel), 8(3): 83). Although chemical lysis and thermal lysis are described herein, any suitable method of cell lysis may be used.

### Chemical Lysis

In some embodiments, cell lysis is performed by exposing a sample to one or more lysis reagents. In some embodiments, the one or more lysis reagents comprise one or more detergents. Without wishing to be bound by a particular theory, a detergent may solubilize membrane proteins and rupture the cell membrane by disrupting interactions between lipids and/or proteins. Non-limiting examples of suitable detergents include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40. In some embodiments, the one or more lysis reagents comprise one or more enzymes. Non-limiting examples of suitable enzymes include lysozyme, lysostaphin, zymolase, cellulase, protease, and glycanase. In some embodiments, the one or more lysis reagents comprise a pH-changing reagent (e.g., an acid or base).

In some embodiments, the one or more lysis reagents are active at approximately room temperature (e.g., 20° C.-25° C.). In some embodiments, the one or more lysis reagents are active at elevated temperatures (e.g., at least 37° C., at least 40° C., at least 50° C., at least 60° C., at least 65° C., at least 70° C., at least 80° C., at least 90° C.). In some embodiments, chemical lysis is performed at a temperature in a range from 20° C. to 25° C., 20° C. to 30° C., 20° C. to 37° C., 20° C. to 50° C., 20° C. to 60° C., 20° C. to 65° C., 20° C. to 70° C., 20° C. to 80° C., 20° C. to 90° C., 25° C. to 30° C., 25° C. to 37° C., 25° C. to 50° C., 25° C. to 60° C., 25° C. to 65° C., 25° C. to 70° C., 25° C. to 80° C., 25° C. to 90° C., 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C.

Thermal Lysis

In some embodiments, cell lysis is performed by thermal lysis (e.g., heating a sample). In some cases, exposure of cells to high temperatures can damage the cellular membrane by denaturing membrane proteins, resulting in cell lysis and the release of intracellular material.

In certain instances, thermal lysis is performed by applying a lysis heating protocol comprising heating a sample at one or more temperatures for one or more time periods using any heater described herein. In some embodiments, a lysis heating protocol comprises heating the sample at a first temperature for a first time period. In certain instances, the first temperature is at least 37° C., at least 40° C., at least 50° C., at least 60° C., at least 63.5° C., at least 65° C., at least 70° C., at least 80° C., or at least 90° C. In certain instances, the first temperature is in a range from 37° C. to 50° C., 37° C. to 60° C., 37° C. to 63.5° C., 37° C. to 65° C., 37° C. to 70° C., 37° C. to 80° C., 37° C. to 90° C., 50° C. to 60° C., 50° C. to 63.5° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the first time period is at least 1 minute, at least 2 minutes, at least 3 minutes, at least 4 minutes, at least 5 minutes, at least 10 minutes, at least 15 minutes, at least 20 minutes, at least 30 minutes, at least 40 minutes, at least 50 minutes, at least 55 minutes, or at least 60 minutes. In certain instances, the first time period is in a range from 1 to 3 minutes, 1 to 5 minutes, 1 to 10 minutes, 1 to 15 minutes, 1 to 20 minutes, 1 to 30 minutes, 1 to 30 minutes, 1 to 40 minutes, 1 to 50 minutes, 1 to 55 minutes, 1 to 60 minutes, 3 to 5 minutes, 3 to 10 minutes, 3 to 15 minutes, 3 to 20 minutes, 3 to 30 minutes, 3 to 40 minutes, 3 to 50 minutes, 3 to 55 minutes, 3 to 60 minutes, 5 to 10 minutes, 5 to 15 minutes, 5 to 20 minutes, 5 to 30 minutes, 5 to 40 minutes, 5 to 50 minutes, 5 to 55 minutes, 5 to 60 minutes, 10 to 20 minutes, 10 to 30 minutes, 10 to 40 minutes, 10 to 50 minutes, 10 to 55 minutes, 10 to 60 minutes, 20 to 30 minutes, 20 to 40 minutes, 20 to 50 minutes, 20 to 55 minutes, 20 to 60 minutes, 30 to 40 minutes, 30 to 50 minutes, 30 to 55 minutes, 30 to 60 minutes, 40 to 50 minutes, 40 to 55 minutes, 40 to 60 minutes, or 50 to 60 minutes.

In some embodiments, a lysis heating protocol comprises heating the sample at a second temperature for a second time period. In certain instances, the second temperature is at least 37° C., at least 40° C., at least 50° C., at least 60° C., at least 63.5° C., at least 65° C., at least 70° C., at least 80° C., or at least 90° C. In certain instances, the second temperature is in a range from 37° C. to 50° C., 37° C. to 60° C., 37° C. to 63.5° C., 37° C. to 65° C., 37° C. to 70° C., 37° C. to 80° C., 37° C. to 90° C., 50° C. to 60° C., 50° C. to 63.5° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the second time period is at least 1 minute, at least 2 minutes, at least 3 minutes, at least 4 minutes, at least 5 minutes, at least 10 minutes, at least 15 minutes, at least 20 minutes, at least 30 minutes, at least 40 minutes, at least 50 minutes, at least 55 minutes, or at least 60 minutes. In certain instances, the second time period is in a range from 1 to 3 minutes, 1 to 5 minutes, 1 to 10 minutes, 1 to 15 minutes, 1 to 20 minutes, 1 to 30 minutes, 1 to 30 minutes, 1 to 40 minutes, 1 to 50 minutes, 1 to 55 minutes, 1 to 60 minutes, 3 to 5 minutes, 3 to 10 minutes, 3 to 15 minutes, 3 to 20 minutes, 3 to 30 minutes, 3 to 40 minutes, 3 to 50 minutes, 3 to 55 minutes, 3 to 60 minutes, 5 to 10 minutes, 5 to 15 minutes, 5 to 20 minutes, 5 to 30 minutes, 5 to 40 minutes, 5 to 50 minutes, 5 to 55 minutes, 5 to 60 minutes, 10 to 20 minutes, 10 to 30 minutes, 10 to 40 minutes, 10 to 50 minutes, 10 to 55 minutes, 10 to 60 minutes, 20 to 30 minutes, 20 to 40 minutes, 20 to 50 minutes, 20 to 55 minutes, 20 to 60 minutes, 30 to 40 minutes, 30 to 50 minutes, 30 to 55 minutes, 30 to 60 minutes, 40 to 50 minutes, 40 to 55 minutes, 40 to 60 minutes, or 50 to 60 minutes.

In one non-limiting embodiment, the first temperature is in a range from 37° C. to 50° C. (e.g., about 37° C.) and the first time period is in a range from 1 minute to 5 minutes (e.g., about 3 minutes), and the second temperature is in a range from 60° C. to 70° C. (e.g., about 65° C.) and the second time period is in a range from 5 minutes to 15 minutes (e.g., about 10 minutes).

In some embodiments, a lysis heating protocol may comprise heating a sample at one or more additional temperatures for one or more additional time periods.

### Nucleic Acid Extraction and Purification

Cell lysis generally results in the release of all intracellular materials, including both nucleic acids and other material (e.g., proteins, lipids and other contaminants), from a cell. Following cell lysis, in some embodiments a step of nucleic acid extraction and/or purification is performed to separate the nucleic acid molecules from other cellular material. Methods of nucleic acid extraction and purification include solution-based methods and solid-phase methods.

In some embodiments, a method of nucleic acid extraction and/or purification is a solution-based method. Such methods may comprise mixing lysed sample material with solutions of reagents for purifying RNA and/or DNA. Solution-based methods of nucleic acid extraction and/or purification include guanidinium thiocyanate-phenol-chloroform extraction, cetyltrimethylammonium bromide extraction, Chelex® extraction, alkaline extraction, and cesium chloride gradient centrifugation (with ethidium bromide).

In some embodiments, a method of nucleic acid extraction and/or purification is a solid-phase method. Such methods extract nucleic acid molecules from other cellular material by causing nucleic acids to selectively bind to solid supports, such as beads (e.g., magnetic beads coated with silica), ion-exchange resins, or other materials.

In certain embodiments, a solution containing a chaotropic agent is added to the lysed sample material. Chaotropic agents generally refer to molecules that disrupt hydrogen bonding between water molecules and render nucleic acid molecules less soluble and more likely to bind to solid supports. In some embodiments, the lysed sample material (with or without a chaotropic agent) is brought into contact with a solid support (e.g., beads, resins, or other solid supports). In some cases, the solid support is washed with an alcohol to remove undesired cellular material and other contaminants from the solid support. In some cases, bound nucleic acid molecules are subsequently eluted from the solid support. Elution may, in some embodiments, be accomplished by washing the solid supports with a liquid that re-solubilizes the nucleic acids, thereby freeing the DNA from the support. Solid-phase extraction methods may utilize or comprise spin columns, beads (e.g., magnetic beads), automated nucleic acid extraction systems, liquid handling robots, lab-on-a-chip cartridges, and/or microfluidics.

Other embodiments of the present disclosure do not require a step of nucleic acid extraction and/or purification to separate the nucleic acid molecules from other cellular material. In such embodiments, described elsewhere herein, the nucleic acid molecules of the sample are reverse-transcribed to cDNA and subsequently amplified directly from the specimen in the buffer (e.g., without the need for a separate nucleic acid extraction and purification step).

### Decontamination

In some embodiments, a diagnostic test or system comprises one or more reagents configured to reduce contamination. In certain embodiments, for example, isothermal amplification methods described herein may include a modified nucleotide (e.g., deoxyuridine triphosphate (dUTP)) along with naturally occurring nucleotides (e.g., deoxyadenosine triphosphate (dATP), deoxyguanosine triphosphate (dGTP), deoxycytidine triphosphate (dCTP), and thymidine triphosphate (dTTP)) during amplification. As a result, amplicons may incorporate the modified nucleotide (e.g., dUTP). In such embodiments, a subsequent test or system may comprise a uracil-DNA glycosylase (UDG). In some cases, activated UDG may degrade any existing uracil-comprising amplicons present (e.g., due to contamination from a prior test) prior to amplification.

Without wishing to be bound by a particular theory, it is thought that the addition of dUTP and UDG may advantageously reduce or eliminate potential contamination between samples. In the absence of dUTP and UDG, amplicons may aerosolize and contaminate future tests, potentially resulting in false positive test results. The use of UDG (e.g., thermolabile UDG) may prevent carryover contamination by specifically degrading products that have already been amplified (i.e., any existing amplicons), leaving the unamplified (new) sample untouched and ready for amplification. Using this method, tests may be performed sequentially in the same tube and/or in the same area.

In some embodiments, a diagnostic method may comprise a first heating step at a first temperature for a first period of time. In certain instances, the first temperature is at least 30° C., at least 35° C., at least 37° C., at least 40° C., at least 45° C., or at least 50° C. In certain instances, the first temperature is 37° C. In some embodiments, the first temperature is in a range from 30° C. to 37° C., 30° C. to 40° C., 30° C. to 45° C., 30° C. to 50° C., 35° C. to 37° C., 35° C. to 40° C., 35° C. to 45° C., 35° C. to 50° C., 40° C. to 45° C., 40° C. to 50° C., and 45° C. to 50° C. In certain instances, the first time period is at least 1 minute, at least 2 minutes, at least 3 minutes, at least 4 minutes, at least 5 minutes, or at least 10 minutes. In some embodiments, the first time period is 3 minutes. In certain instances, the first time period is in a range from 1 to 3 minutes, 1 to 5 minutes, 1 to 10 minutes, 3 to 5 minutes, 3 to 10 minutes, or 5 to 10 minutes. In a particular, non-limiting embodiment, the first temperature is 37° C. and the first time period is 3 minutes.

In some embodiments, a diagnostic method further comprises a second heating step at a second temperature for a second period of time. In some embodiments, the second heating step may denature and therefore inactivate the UDG enzyme prior to performing any amplification steps. In some embodiments, the second heating step may correspond to a thermal lysis and/or amplification heating step. In some embodiments, the second temperature is at least 37° C., at least 40° C., at least 45° C., at least 50° C., at least 55° C., at least 60° C., at least 63.5° C., at least 65° C., at least 70° C., at least 80° C., at least 90° C., at least 95° C., or at least 100° C. In some embodiments, the second temperature is in a range from 37° C. to 50° C., 37° C. to 60° C., 37° C. to 63.5° C., 37° C. to 65° C., 37° C. to 70° C., 37° C. to 80° C., 37° C. to 90° C., 37° C. to 95° C., 37° C. to 100° C., 50° C. to 60° C., 50° C. to 63.5° C., 50° C. to 65° C., 50° C. to 70° C., 50° C. to 80° C., 50° C. to 90° C., 50° C. to 95° C., 50° C. to 100° C., 60° C. to 63.5° C., 60° C. to 65° C., 60° C. to 70° C., 60° C. to 80° C., 60° C. to 90° C., 60° C. to 95° C., 60° C. to 100° C., 65° C. to 80° C., 65° C. to 90° C., 65° C. to 95° C., 65° C. to 100° C., 70° C. to 80° C., 70° C. to 90° C., 70° C. to 95° C., 70° C. to 100° C., 80° C. to 90° C., 80° C. to 95° C., 80° C. to 100° C., or 90° C. to 100° C. In certain instances, the second time period is at least 1 minute, at least 2 minutes, at least 3 minutes, at least 4 minutes, at least 5 minutes, at least 10 minutes, at least 15 minutes, at least 20 minutes, at least 30 minutes, at least 40 minutes, at least 50 minutes, at least 55 minutes, or at least 60 minutes. In certain instances, the second time period is in a range from 1 to 3 minutes, 1 to 5 minutes, 1 to 10 minutes, 1 to 15 minutes, 1 to 20 minutes, 1 to 30 minutes, 1 to 40 minutes, 1 to 50 minutes, 1 to 55 minutes, 1 to 60 minutes, 3 to 5 minutes, 3 to 10 minutes, 3 to 15 minutes, 3 to 20 minutes, 3 to 30 minutes, 3 to 40 minutes, 3 to 50 minutes, 3 to 55 minutes, 3 to 60 minutes, 5 to 10 minutes, 5 to 15 minutes, 5 to 20 minutes, 5 to 30 minutes, 5 to 40 minutes, 5 to 50 minutes, 5 to 55 minutes, 5 to 60 minutes, 10 to 20 minutes, 10 to 30 minutes, 10 to 40 minutes, 10 to 50 minutes, 10 to 55 minutes, 10 to 60 minutes, 20 to 30 minutes, 20 to 40 minutes, 20 to 50 minutes, 20 to 55 minutes, 20 to 60 minutes, 30 to 40 minutes, 30 to 50 minutes, 30 to 55 minutes, 30 to 60 minutes, 40 to 50 minutes, 40 to 55 minutes, 40 to 60 minutes, or 50 to 60 minutes.

### Nucleic Acid Amplification

Following cell lysis and, in some embodiments, nucleic acid extraction and purification, one or more target nucleic acids (e.g., a nucleic acid of a target pathogen) are amplified. Methods of amplifying ribonucleic acids (RNA) and deoxyribonucleic acids (DNA) are specifically contemplated herein. In certain instances, for example, a target pathogen is an RNA virus (e.g., a coronavirus, an influenza virus), and therefore has RNA as its genetic material. In some such cases, the target pathogen's RNA may need to be reverse transcribed to DNA prior to amplification.

In some embodiments, reverse transcription is performed by exposing lysate to one or more reverse transcription reagents. In certain instances, the one or more reverse transcription reagents comprise a reverse transcriptase, a DNA-dependent polymerase, and/or a ribonuclease (RNase). A reverse transcriptase generally refers to an enzyme that transcribes RNA to complementary DNA (cDNA) by polymerizing deoxyribonucleotide triphosphates (dNTPs). An RNase generally refers to an enzyme that catalyzes the degradation of RNA. In some cases, an RNase may be used to digest RNA from an RNA-DNA hybrid. In some embodiments, the reverse transcriptase and DNA-dependent polymerase are inactive at room temperature (e.g., are “warm start”, e.g., require a step of heating in order to activate).

In some embodiments, DNA may be amplified according to any nucleic acid amplification method known in the art. In some embodiments, amplification is performed under essentially isothermal conditions. In some embodiments, the nucleic acid amplification method is an isothermal amplification method. Isothermal amplification methods include, but are not limited to, loop-mediated isothermal amplification (LAMP), recombinase polymerase amplification (RPA), nicking enzyme amplification reaction (NEAR), nucleic acid sequence-based amplification (NASBA), strand displacement amplification (SDA), helicase-dependent amplification (HDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), and whole genome amplification (WGA). In one embodiment, the nucleic acid amplification method is loop-mediated isothermal amplification (LAMP). In another embodiment, the nucleic acid amplification method is recombinase polymerase amplification (RPA). In another embodiment, the nucleic acid amplification method is nicking enzyme amplification reaction (NEAR). In some embodiments, the nucleic acid amplification method consists of applying one or more nucleic acid amplification reagents to a sample.

Loop-Mediated Isothermal Amplification (LAMP)

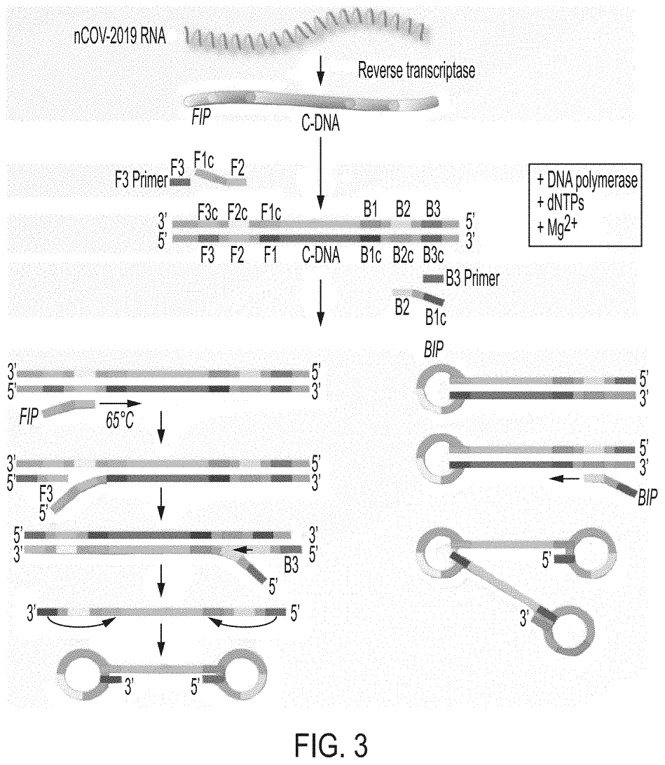

In some embodiments, the nucleic acid amplification method is an isothermal amplification method comprising loop-mediated isothermal amplification (LAMP). Accordingly, in some embodiments, the nucleic acid amplification reagents are LAMP reagents. LAMP generally refers to a DNA amplification technique originally developed by Notomi, et al., (Nucleic Acids Research 28:E63 (2000)) in which a target nucleic acid is amplified using at least four primers through the creation of a series of stem-loop structures. Due to its use of multiple primers, LAMP may be highly specific for a target nucleic acid sequence. FIG. 3 is a schematic illustration of an exemplary LAMP amplification method.

LAMP employs a primer set of four essential primers, termed the forward inner primer (FIP), backward inner primer (BIP), forward outer primer (also known as forward displacement primer) (F3), and backward outer primer (also known as backward displacement primer) (B3). The 4-primer LAMP method, using FIP, BIP, F3, and B3 primers, is the basic form of LAMP that was originally described for isothermal nucleic acid amplification. In some embodiments, the LAMP reagents comprise four or more primers. In certain embodiments, the four or more primers comprise a FIP, a BIP, a F3, and a B3 primer. In some cases, the four or more primers target at least six specific regions of a target gene.

Additionally, two optional primers, a forward loop primer (Loop F or LF) and a backward loop primer (Loop B or LB), can also be included in the LAMP reaction. In certain cases, the loop primers target cyclic structures formed during amplification and can accelerate amplification. One or both of the LF and LB primers may be included; the addition of both loop primers significantly accelerates LAMP. In some embodiments, the LAMP reagents further comprise an LF primer and/or an LB primer.

In some cases, LAMP primers may be designed for each target nucleic acid a diagnostic device is configured to detect. For example, a diagnostic device configured to detect a first target nucleic acid (e.g., a nucleic acid of SARS-CoV-2) and a second target nucleic acid (e.g., a nucleic acid of an influenza virus) may comprise a first set of LAMP primers directed to the first target nucleic acid and a second set of LAMP primers directed to the second target nucleic acid. In some embodiments, the LAMP primers may be designed by alignment and identification of conserved sequences in a target pathogen (e.g., using Clustal X or a similar program) and then using a software program (e.g., PrimerExplorer). The specificity of different candidate primers may be confirmed using a BLAST search of the GenBank nucleotide database. Primers may be synthesized using any method known in the art.

In certain embodiments, the target pathogen is SARS-CoV-2. In some cases, primers for amplification of a SARS-CoV-2 nucleic acid sequence are selected from regions of the virus's nucleocapsid (N) gene, envelope (E) gene, membrane (M) gene, and/or spike (S) gene. In some instances, primers were selected from regions of the SARS-CoV-2 nucleocapsid (N) gene to maximize inclusivity across known SARS-CoV-2 strains and minimize cross-reactivity with related viruses and genomes that may be presence in the sample. In some embodiments, six SARS-CoV-2 LAMP primers target the Open Reading Frame 1ab (orf1ab) region of the SARS-CoV-2 genome. In certain embodiments, the six SARS-CoV-2 LAMP primers comprise SEQ ID NOS. 1-6. In certain embodiments, the six SARS-CoV-2 LAMP primers comprise SEQ ID Nos. 9-14. In some cases, more than six LAMP primers may be used. In certain instances, for example, eight primers may be used. In some embodiments, the eight primers comprise SEQ ID NOS. 1-8.

Exemplary LAMP primers for detection of a SARS-CoV-2 nucleic acid sequence are provided in Table 1 below.

TABLE 1
Exemplary LAMP Primers (SARS-CoV-2)

| | | SEQ ID |
|---|---|---|
| Primer | Sequence (5′ to 3′) | NO: |
| F3_Set1 | CGGTGGACAAATTGTCAC | 1 |
| B3_Set1 | CTTCTCTGGATTTAACACACTT | 2 |
| Loop F_Set1 | TTACAAGCTTAAAGAATGTCTGAACACT | 3 |
| Loop F_Set1 | /56-FAM/TTACAAGCTTAAAGAATGTCTGAACACT | 3 |
| conjugated to | | |
| label | | |
| Loop B_Set1 | TTGAATTTAGGTGAAACATTTGTCACG | 4 |
| Loop B_Set1 | /5Biosg/TTGAATTTAGGTGAAACATTTGTCAC G | 4 |
| conjugated to | | |
| label | | |
| FIP1_Set1 | TCAGCACACAAAGCCAAAAATTTATTTTTCTGTGCAAAG | 5 |
| | GAAATTAAGGAG | |
| BIP1_Set1 | TATTGGTGGAGCTAAACTTAAAGCCTTTTCTGTACAATC | 6 |
| | CCTTTGAGTG | |
| FIP2_Set1 | TCAGCACACAAAGCCAAAAATTTATCTGTGCAAAGGAA | 7 |
| | ATTAAGGAG | |
| BIP2_Set1 | TATTGGTGGAGCTAAACTTAAAGCCCTGTACAATCCCTT | 8 |
| | TGAGTG | |
| F3_Set2 | TGCTTCAGTCAGCTGATG | 9 |
| B3_Set2 | TTAAATTGTCATCTTCGTCCTT | 10 |
| FIP_Set2 | TCAGTACTAGTGCCTGTGCCCACAATCGTTTTTAAACGG | 11 |
| | GT | |
| BIP_Set2 | TCGTATACAGGGCTTTTGACATCTATCTTGGAAGCGACA | 12 |
| | ACAA | |
| Loop F_Set2 | CTGCACTTACACCGCAA | 13 |
| Loop B_Set2 | GTAGCTGGTTTTGCTAAATTCC | 14 |

In some embodiments, the LAMP reagents comprise a FIP and a BIP for one or more target nucleic acids. In some embodiments, the FIP and BIP each have a sequence that is at least 90%, at least 95%, at least 96%, at least 97%, at least 98%, at least 99%, at least 99.5%, or 100% identical to a primer sequence provided in Table 1. In some embodiments, the concentrations of FIP and BIP are each at least 0.5 μM, at least 0.6 μM, at least 0.7 μM, at least 0.8 μM, at least 0.9 μM, at least 1.0 μM, at least 1.1 μM, at least 1.2 μM, at least 1.3 μM, at least 1.4 μM, at least 1.5 μM, at least 1.6 μM, at least 1.7 μM, at least 1.8 μM, at least 1.9 μM, or at least 2.0 μM. In some embodiments, the concentrations of FIP and BIP are each in a range from 0.5 μM to 1 μM, 0.5 μM to 1.5 μM, 0.5 μM to 2.0 μM, 1 μM to 1.5 μM, 1 μM to 2 μM, or 1.5 μM to 2 μM.

In some embodiments, the LAMP reagents comprise an F3 primer and a B3 primer for one or more target nucleic acids. In some embodiments, the F3 primer and the B3 primer each have a sequence that is at least 90%, at least 95%, at least 96%, at least 97%, at least 98%, at least 99%, at least 99.5%, or 100% identical to a primer sequence provided in Table 1. In some embodiments, the concentrations of the F3 primer and the B3 primer are each at least 0.05 μM, at least 0.1 μM, at least 0.15 μM, at least 0.2 μM, at least 0.25 μM, at least 0.3 μM, at least 0.35 μM, at least 0.4 μM, at least 0.45 μM, or at least 0.5 μM. In some embodiments, the concentrations of the F3 primer and the B3 primer are each in a range from 0.05 μM to 0.1 μM, 0.05 μM to 0.2 μM, 0.05 μM to 0.3 μM, 0.05 μM to 0.4 μM, 0.05 μM to 0.5 μM, 0.1 μM to 0.2 μM, 0.1 μM to 0.3 μM, 0.1 μM to 0.4 μM, 0.1 μM to 0.5 μM, 0.2 μM to 0.3 μM, 0.2 μM to 0.4 μM, 0.2 μM to 0.5 μM, 0.3 μM to 0.4 μM, 0.3 μM to 0.5 μM, or 0.4 μM to 0.5 μM.

In some embodiments, the LAMP reagents comprise a forward loop primer and a backward loop primer for one or more target nucleic acids. In some embodiments, the forward loop primer and the backward loop primer each have a sequence that is at least 90%, at least 95%, at least 96%, at least 97%, at least 98%, at least 99%, at least 99.5%, or 100% identical to a primer sequence provided in Table 1. In some embodiments, the concentrations of the forward loop primer and the backward loop primer are each at least 0.1 μM, at least 0.2 μM, at least 0.3 μM, at least 0.4 μM, at least 0.5 μM, at least 0.6 μM, at least 0.7 μM, at least 0.8 μM, at least 0.9 μM, or at least 1.0 μM. In some embodiments, the concentrations of the forward loop primer and the backward loop primer are each in a range from 0.1 μM to 0.2 μM, 0.1 μM to 0.5 μM, 0.1 μM to 0.8 μM, 0.1 μM to 1.0 μM, 0.2 μM to 0.5 μM, 0.2 μM to 0.8 μM, 0.2 μM to 1.0 μM, 0.3 μM to 0.5 μM, 0.3 μM to 0.8 μM, 0.3 μM to 1.0 μM, 0.4 μM to 0.8 μM, 0.4 μM to 1.0 μM, 0.5 μM to 0.8 μM, 0.5 μM to 1.0 μM, or 0.8 μM to 1.0 μM.

In some embodiments, the LAMP reagents comprise LAMP primers designed to simultaneously amplify a human or animal nucleic acid that is not associated with a pathogen, a cancer cell, or a contaminant in a multiplexed reaction. In some such embodiments, the human or animal nucleic acid may act as a control (e.g., an internal sample processing control). For example, successful amplification and detection of the control nucleic acid may indicate that a sample was properly collected and the diagnostic test was properly run. On the other hand, failure to detect the control nucleic acid may indicate one or more of the following: improper specimen collection resulting in the lack of sufficient human sample material, improper extraction/purification of nucleic acid from the sample, ineffective inhibition of RNAse in the sample, improper assay set up and execution, and/or reagent or equipment malfunction.

In some instances, the control nucleic acid is a nucleic acid sequence encoding human RNase P. In some embodiments, the RPA reagents comprise primers (e.g., forward primers, reverse primers) and probes configured to detect a nucleic acid sequence encoding human RNase P.

In some embodiments, the control nucleic acid is a nucleic acid sequence encoding human RNase P. Exemplary LAMP primers for RNase P are shown in Table 2. In some instances, the one or more LAMP reagents comprise at least four primers that each have a sequence that is at least 90%, at least 95%, at least 96%, at least 97%, at least 98%, at least 99%, at least 99.5%, or 100% identical to a primer sequence provided in Table 2.

TABLE 2
Exemplary RNase P Primers

| | | SEQ ID |
|---|---|---|
| Primer | Sequence (5′ to 3′) | NO: |
| F3 | TTGATGAGCTGGAGCCA | 15 |
| B3 | CACCCTCAATGCAGAGTC | 16 |
| FTP | GTGTKACCCTGAAGACTCGGTTTTAGCC | 17 |
| | ACTGACTCGGATC | |
| BIP | CCTCCGTGATATGGCTCTTCGTTTTTTT | 18 |
| | CTTACATGGCTCTGGTC | |
| Loop F | ATGTGGATGGCTGAGTTGTT | 19 |
| Loop F | /5DigN/ATGTGGATGGCTGAGTTGTT | 19 |
| conjugated to | | |
| label | | |
| Loop B | CATGCTGAGTACTGGACCTC | 20 |
| Loop B | /5Biosg/CATGCTGAGTACTGGACCTC | 20 |
| conjugated to | | |
| label | | |
| Quencher | CAGCCATCCACAT-BHQ1 | 21 |

In some embodiments, one or more LAMP primers (e.g., a target nucleic acid LAMP primer, or a control nucleic acid LAMP primer) are conjugated to a label. Conjugation of one or more LAMP primers to a label is desirable in embodiments to visualize readout results, for example on a lateral flow assay strip. Non-limiting examples of suitable labels include biotin, streptavidin, fluorescein isothiocyanate (FITC), fluorescein amidite (FAM), fluorescein, and digoxigenin (DIG). In some cases, labeling one or more LAMP primers may result in labeled amplicons, which may facilitate detection (e.g., via a lateral flow assay). In some embodiments, one or more LAMP primers are conjugated to FAM. In some embodiments, one or more LAMP primers are conjugated to biotin. In some embodiments, one of the six LAMP primers is conjugated to FAM, and another one of the six LAMP primers is conjugated to biotin. In such embodiments, successful on-target amplification involving all six primers generates amplicons labeled with both FAM and biotin. In some embodiments, one of the six LAMP primers is conjugated to DIG. In some embodiments, one of the six LAMP primers is conjugated to DIG, and another LAMP primer is conjugated to biotin. In such embodiments, successful on-target amplification involving all six primers generates amplicons labeled with both DIG and biotin. In certain embodiments, the label is a fluorescent label. In some instances, the fluorescent label is associated with a quenching moiety that prevents the fluorescent label from signaling until the quenching moiety is removed. In certain embodiments, a LAMP primer is labeled with two or more labels.

In some embodiments, the LAMP reagents comprise a DNA polymerase with high strand displacement activity. Non-limiting examples of suitable strand-displacing DNA polymerases include a DNA polymerase long fragment (LF) of a thermophilic bacterium, such as Bacillus stearothermophilus (Bst), Bacillus Smithii (Bsm), Geobacillus sp. M (GspM), or Thermodesulfatator indicus (Tin), or a Taq DNA polymerase. In certain embodiments, the DNA polymerase is Bst LF DNA polymerase, GspM LF DNA polymerase, GspSSD LF DNA polymerase, Tin exo-LF DNA polymerase, or SD DNA polymerase. In each case, the DNA polymerase may be a wild type or mutant polymerase.

In some embodiments, the concentration of the DNA polymerase is at least 0.1 U/μL, at least 0.2 U/μL, at least 0.3 U/μL, at least 0.4 U/μL, at least 0.5 U/μL, at least 0.6 U/μL, at least 0.7 U/μL, at least 0.8 U/μL, at least 0.9 U/μL, or at least 1.0 U/μL. In some embodiments, the concentration of the DNA polymerase is in a range from 0.1 U/μL to 0.5 U/μL, 0.1 U/μL to 1.0 U/μL, 0.2 U/μL to 0.5 U/μL, 0.2 U/μL to 1.0 U/μL, or 0.5 U/μL to 1.0 U/μL.

In some embodiments, the LAMP reagents comprise deoxyribonucleotide triphosphates (“dNTPs”). In certain embodiments, the LAMP reagents comprise deoxyadenosine triphosphate (“dATP”), deoxyguanosine triphosphate (“dGTP”), deoxycytidine triphosphate (“dCTP”), and deoxythymidine triphosphate (“dTTP”). In certain embodiments, the concentration of each dNTP (i.e., dATP, dGTP, dCTP, dTTP) is at least 0.5 mM, at least 0.6 mM, at least 0.7 mM, at least 0.8 mM, at least 0.9 mM, at least 1.0 mM, at least 1.1 mM, at least 1.2 mM, at least 1.3 mM, at least 1.4 mM, at least 1.5 mM, at least 1.6 mM, at least 1.7 mM, at least 1.8 mM, at least 1.9 mM, or at least 2.0 mM. In some embodiments, the concentration of each dNTP is in a range from 0.5 mM to 1.0 mM, 0.5 mM to 1.5 mM, 0.5 mM to 2.0 mM, 1.0 mM to 1.5 mM, 1.0 mM to 2.0 mM, or 1.5 mM to 2.0 mM.

In some embodiments, the LAMP reagents comprise magnesium sulfate (MgSO₄). In certain embodiments, the concentration of MgSO₄ is at least 1 mM, at least 2 mM, at least 3 mM, at least 4 mM, at least 5 mM, at least 6 mM, at least 7 mM, at least 8 mM, at least 9 mM, or at least 10 mM. In certain embodiments, the concentration of MgSO₄ is in a range from 1 mM to 2 mM, 1 mM to 5 mM, 1 mM to 8 mM, 1 mM to 10 mM, 2 mM to 5 mM, 2 mM to 8 mM, 2 mM to 10 mM, 5 mM to 8 mM, 5 mM to 10 mM, or 8 mM to 10 mM.

In some embodiments, the LAMP reagents comprise betaine. In certain embodiments, the concentration of betaine is at least 0.1 M, at least 0.2 M, at least 0.3 M, at least 0.4 M, at least 0.5 M, at least 0.6 M, at least 0.7 M, at least 0.8 M, at least 0.9 M, at least 1.0 M, at least 1.1 M, at least 1.2 M, at least 1.3 M, at least 1.4 M, or at least 1.5 M. In certain embodiments, the concentration of betaine is in a range from 0.1 M to 0.2 M, 0.1 M to 0.5 M, 0.1 M to 0.8 M, 0.1 M to 1.0 M, 0.1 M to 1.2 M, 0.1 M to 1.5 M, 0.2 M to 0.5 M, 0.2 M to 0.8 M, 0.2 M to 1.0 M, 0.2 M to 1.2 M, 0.2 M to 1.5 M, 0.5 M to 0.8 M, 0.5 M to 1.0 M, 0.5 M to 1.2 M, 0.5 M to 1.5 M, 0.8 M to 1.0 M, 0.8 M to 1.2 M, 0.8 M to 1.5M, 1.0 M to 1.2 M, or 1.0 M to 1.5M.

In some embodiments, a modified LAMP protocol is used. For example, LAMP may be performed using small labels (e.g., labeled haptens) for the undiluted (direct) detection of LAMP products (e.g., amplicons) on lateral flow strips. Smaller analytes (e.g., haptens) employed in conjunction with formamidopyrimidine DNA glycosylase (Fpg) have been shown to permit direct (undiluted) lateral flow detection of amplicons (Powell et al., Analytical Biochem., 2018, 543(15): 108-115). In some embodiments, dual hapten probes are used in the LAMP method.

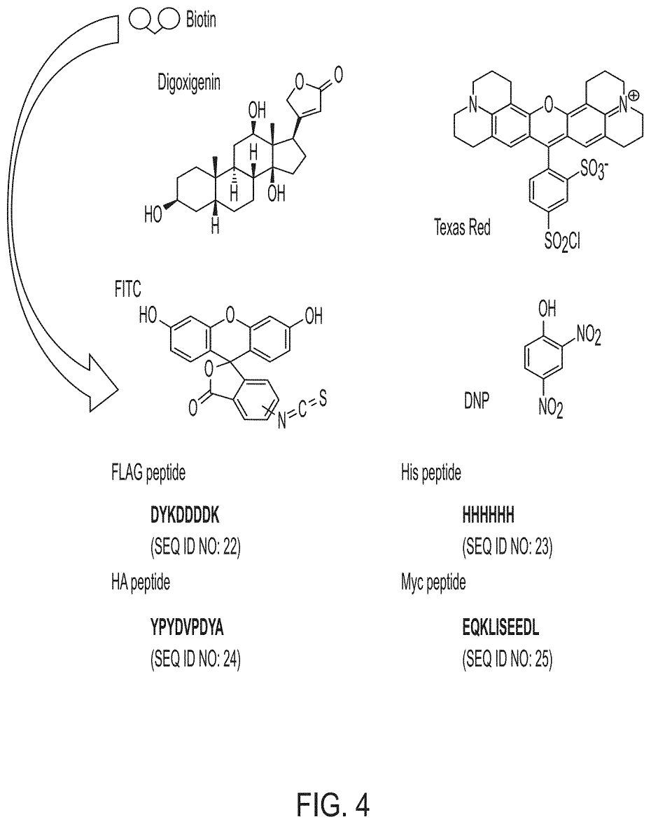

In some embodiments, the dual hapten probes comprise two haptens joined by a linker, such as a lysine residue. The smaller probes (e.g., dual hapten probes) may be separated from the reaction until after amplification has occurred. Then, an Fpg probe against a specific amplicon target conjugated to the dual-hapten label may be released. The probe may bind to the specific amplicon target, and Fpg may cleave the dual-hapten label. Since the dual-hapten label is relatively small, it may be able to readily advance to the lateral flow strip. Then, the label may be detected by any means known in the art, such as with a sandwich immunoassay on a lateral flow strip (e.g., a lateral flow test comprising an antibody against one of the analytes and gold particles comprising antibodies against the second analyte). Non-limiting examples of dual haptens for labeling are shown in FIG. 4 and include biotin in combination with digoxigenin, FITC, Texas Red, dinitrophenol (DNP), FLAG peptide (DYKDDDDK; SEQ ID NO: 22), His peptide (HHHHHH; SEQ ID NO: 23), HA peptide (YPYDVPDYA; SEQ ID NO: 24), and Myc peptide (EQKLISEEDL; SEQ ID NO: 25).

Recombinase Polymerase Amplification (RPA)

In some embodiments, the nucleic acid amplification method is an isothermal amplification method comprising recombinase polymerase amplification (RPA). Accordingly, in some embodiments the nucleic acid amplification reagents are RPA reagents. RPA generally refers to a method of amplifying a target nucleic acid using a recombinase, a single-stranded DNA binding protein, and a strand-displacing polymerase.

In some embodiments, the RPA reagents comprise a probe, a forward primer, and a reverse primer. The probe, forward primer, and reverse primer may be designed for each target nucleic acid a diagnostic device is configured to detect.

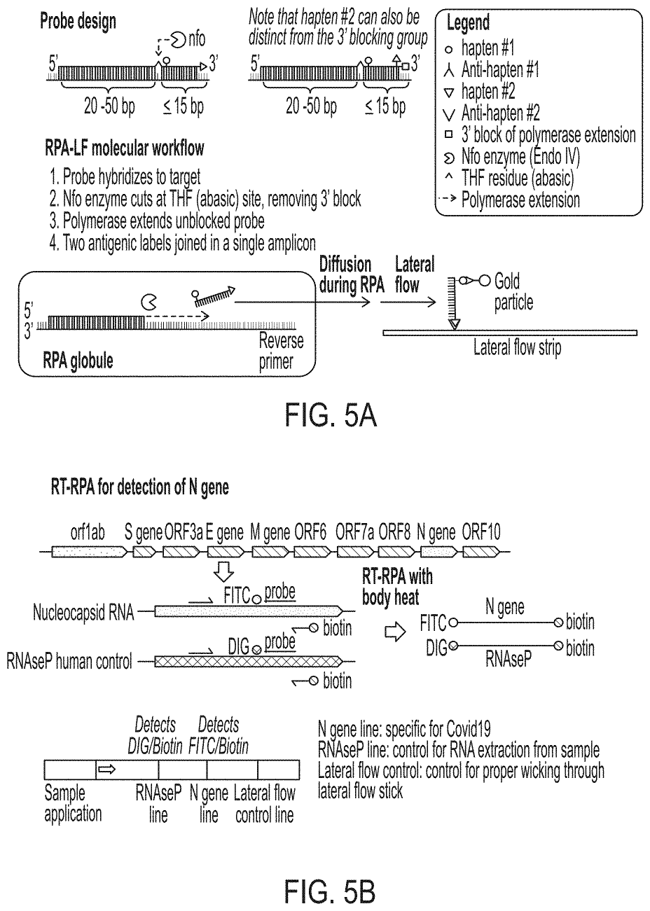

In some embodiments, RPA is performed with a single-stranded DNA probe comprising a 5′ initial hybridization region, an abasic site, a detection region downstream of the abasic site, and a 3′ blocking group. In some embodiments, the initial hybridization region is located toward the 5′ end of the probe and the detection region is located toward the 3′ end of the probe. In one embodiment, illustrated in FIG. 5A, the probe comprises, from 5′ to 3′, the initial hybridization region, the abasic site, the detection region, and the 3′ blocking group.

As illustrated in FIG. 5A, the probe binds its intended target in the sample, forming duplex DNA. The single-stranded probe is long enough (≥15 bp) that it remains trapped within RPA globules. After the probe binds its target and forms a duplex, a DNA repair enzyme or structure-specific endonuclease or exonuclease creates a single base pair gap at the abasic site of the probe. Non-limiting examples of DNA repair enzymes include mutH, mutL, mutM, mutS, mutY, dam, thymidine DNA glycosylase (TDG), uracil DNA glycosylase, formamidopyrimidine DNA glycosylase, AlkA, MLH1, MSH2, MSH3, MSH6, FEN1 (RAD27), dnaQ (mutD), polC (dnaE), or combinations thereof. Examples of endonucleases that recognize an abasic (e.g., apurinic or apyrimidinic) site include, but are not limited to, APE 1 (or HAP 1 or Ref-1), Endonuclease III, Endonuclease IV, T4 endonuclease V, Endonuclease VIII, Fpg, and Hogg1. Examples of exonucleases include, but are not limited to, Exonuclease I, Exonuclease III Exonuclease V, RecJ exonuclease, Exonuclease T, S1 nuclease, P1 nuclease, mung bean nuclease, T4 DNA polymerase, and CEL I nuclease.

Cleavage at the abasic site yields the duplexed 5′ initial hybridization region and a short (<15 bp) single-stranded oligonucleotide from the 3′ end (the detection region), which is removed from the duplex when strand displacing polymerases extend from the cleavage site. Exemplary polymerases include, but are not limited to, pol-α, pol-β, poi-δ, poi-ε, E. coli DNA polymerase I Klenow fragment, bacteriophage T4 gp43 DNA polymerase, Bacillus stearothermophilus polymerase I large fragment, Phi-29 DNA polymerase, T7 DNA polymerase, Bacillus subtilis Pol I, E. coli DNA polymerase I, E. coli DNA polymerase II, E. coli DNA polymerase III, E. coli DNA polymerase IV, E. coli DNA polymerase V and derivatives and combinations thereof.

The displaced oligonucleotide is able to diffuse from the RPA globule, owing to its relatively small size (<15 bp). The sample is then assayed and the presence or absence of the oligonucleotide is determined. For example, the sample may be loaded onto a lateral flow strip, where the low viscosity RPA product advances along the strip via capillary action. As described herein, the lateral flow strip comprises immobilized antibodies specific for at least one of the hapten labels on the oligonucleotide, so that the oligonucleotide, if present, will be captured in the correct location (e.g., first test line) on the lateral flow test. The oligonucleotide may then be visualized using a second labeled antibody against a second hapten present on the oligonucleotide (e.g., with a gold-conjugated second antibody).

In certain embodiments, each primer comprises at least 15 base pairs, at least 20 base pairs, at least 25 base pairs, at least 30 base pairs, at least 35 base pairs, at least 40 base pairs, at least 45 base pairs, or at least 50 base pairs. In certain embodiments, each primer comprises 15-20 base pairs, 15-30 base pairs, 15-40 base pairs, 15-50 base pairs, 20-30 base pairs, 20-40 base pairs, 20-50 base pairs, 30-40 base pairs, 30-50 base pairs, or 40-50 base pairs. In some embodiments, each primer does not have any mismatches within 3 base pairs of its 3′ terminus. In some embodiments, each primer comprises 10 or fewer, 9 or fewer, 8 or fewer, 7 or fewer, 6 or fewer, 5 or fewer, 4 or fewer, 3 or fewer, 2 or fewer, 1 or fewer, or no mismatches. In some embodiments, each mismatch is at least 3 base pairs, at least 4 base pairs, at least 5 base pairs, at least 6 base pairs, at least 7 base pairs, at least 8 base pairs, at least 9 base pairs, or at least 10 base pairs from the 3′ terminus. While mismatches more than 3 base pairs away from the 3′ terminus of the primer have been found to be well tolerated in RPA, multiple mismatches within 3 base pairs of the 3′ terminus have been found to inhibit the reaction.

As an illustrative example, in some instances, a first target nucleic acid is a nucleic acid of SARS-CoV-2. RPA typically includes a recombinase agent, which is contacted with a forward and a reverse nucleic acid primer to form a first and a second nucleoprotein primer. The oligonucleotide primers and probes for amplification and detection of SARS-CoV-2 were selected from regions of the virus nucleocapsid (N) gene to maximize inclusivity across known SARS-CoV-2 strains and minimize cross-reactivity with related viruses and genomes likely to be present in the sample. In other embodiments, the oligonucleotide primers and probes are selected from regions of the virus' envelope (E) gene, membrane (M) gene, and/or spike (S) gene. The panel, in some embodiments, is designed for specific detection of the SARS-CoV-2 (one primer/probe set). An additional primer/probe set to detect the human RNase P gene (RP) in control samples and clinical specimens is also included in some embodiments. RT-RPA for detection of the N gene of SARS-CoV-2 is illustrated in FIG. 5B.

Exemplary RPA primers for detection of a nucleic acid sequence from the SARS-CoV-2 nucleocapsid (N) gene are provided in Table 3 below.

TABLE 3
Exemplary Recombination Polymerase
Amplification Primers

| | | SEQ |
|---|---|---|
| | | ID |
| RPA_primer | Sequence | NO: |
| Forward | GTACTGCCACTAAAGCATACAATGTAACAC | 26 |
| Primer | | |
| Reverse | {6-FAM}AATATGCTTATTCAGCAAAATGACTT | 27 |
| Primer | GATCT | |
| Probe | {biotin}CAGACAAGGAACTGATTACA | 28 |
| | AACATTGGCCGCA{dS | |
| | pacer}ATTGCACAATTTGCC{phos} | |
| RPA_fwd_1 | TCTGATAATGGACCCCAAAATCAGCGAAAT | 31 |
| RPA_rev_1 | CTCCATTCTGGTTACTGCCAGTTGAATCTG | 32 |
| RPA_fwd_3 | GCAACTGAGGGAGCCTTGAATACACCAAAA | 33 |
| RPA_rev_3 | TGAGGAAGTTGTAGCACGATTGCAGCATTG | 34 |
| RPA_fwd_2 | AAGGAACTGATTACAAACATTGGCCGCAAA | 35 |
| RPA_rev_2 | TTCCATGCCAATGCGCGACATTCCGAAGAA | 36 |
| RPA_fwd_4 | AAATTTTGGGGACCAGGAACTAATCAGACA | 37 |
| RPA_rev_4 | TGGCACCTGTGTAGGTCAACCACGTTCCCG | 38 |
| Set1_fwd | ACCCCAAAATCAGCGAAATGCACCCCGCATTA | 39 |
| Set1_rev | GTAGAAATACCATCTTGGACTGAGA | 40 |
| Set2_fwd | GTCTGATAATGGACCCCAAAATCAGCGA | 41 |
| Set2_rev | TAGTAGAAATACCATCTTGGACTGAGATCTTT | 42 |
| Set1_probe | AGAATGGAGAACGCAGTGGGGCGCGATCAAAACA | 43 |
| | ACGTCGGCCCC | |
| Set1_probe | AGAATGGAGAACGCAGTGGGGCGCGATCA | 44 |
| v1 | [dSpacer]AACAACGTCGGCCCC[Block] | |
| Set2_probe | CAGTAACCAGAATGGAGAACGCAGTGGGGCGCGA | 45 |
| | TCAAAACAACGTCGGC | |
| Set2_probe | CAGTAACCAGAATGGAGAACGCAGTGGGGCGCGA | 46 |
| v1 | TCA[dSpacer]AACAACGTCGGCC[Block] | |

The primers and probes in Table 3 were designed to incorporate all COVID-19 variants with a 99% threshold. Mismatches more than 3 bp away from the 3′ terminus of the primer were found to be well tolerated in RPA; however, multiple mismatches within 3 bp of the 3′ terminus may inhibit the reaction completely. Therefore, in some embodiments, the primer has at least one mismatch at least 3, 4, 5, 6, 7, 8, 9, 10, or more bp away from the 3′ terminus of the primer. In some embodiments, the primer comprises 1, 2, 3, 4, 5, 6, 7, 8, 9, 10, or more mismatches. In one embodiment, the primer comprises 1 mismatch. In some embodiments, the primer has one mismatch within 3 bp of its 3′ terminus. In some embodiments, the primer does not have a mismatch within 3 bp of its 3′ terminus. The primers, in some embodiments, comprise 18, 19, 20, 21, 22, 23, 24, 25, 26, 27, 28, 29, 30, or more base pairs. In some embodiments, the enzymes used in the amplification (e.g., RPA) do not have any 3′ exonuclease activity (i.e., the enzymes cannot remove the 3′ end of the primer).

In some embodiments, the RPA reagents comprise one or more forward primers. In certain embodiments, at least one forward primer is at least 70%, at least 75%, at least 80%, at least 85%, at least 90%, at least 95%, at least 99%, or 100% identical to SEQ ID NO: 26. In some embodiments, at least one forward primer is at least 1 base pair, at least 2 base pairs, at least 3 base pairs, at least 4 base pairs, or at least 5 base pairs longer or shorter than SEQ ID NO: 26. In some embodiments, one or more forward primers comprise SEQ ID NOs: 31, 33, 35, 37, 39, and/or 41. In some embodiments, one or more forward primers comprise an antigenic tag. In certain embodiments, the concentration of the one or more forward primers is at least 100 nM, at least 200 nM, at least 300 nM, at least 400 nM, at least 500 nM, at least 600 nM, at least 700 nM, at least 800 nM, at least 900 nM, or at least 1000 nM. In certain embodiments, the concentration of the one or more forward primers is in a range from 100 nM to 200 nM, 100 nM to 500 nM, 100 nM to 800 nM, 100 nM to 1000 nM, 200 nM to 500 nM, 200 nM to 800 nM, 200 nM to 1000 nM, 500 nM to 800 nM, 500 nM to 1000 nM, or 800 nM to 1000 nM.

In some embodiments, the RPA reagents comprise one or more reverse primers. In certain embodiments, at least one reverse primer is at least 70%, at least 75%, at least 80%, at least 85%, at least 90%, at least 95%, at least 99%, or 100% identical to SEQ ID NO: 27. In some embodiments, at least one reverse primer is at least 1 base pair, at least 2 base pairs, at least 3 base pairs, at least 4 base pairs, or at least 5 base pairs longer or shorter than SEQ ID NO: 27. In some embodiments, the one or more reverse primers, in some embodiments, comprise SEQ ID NOs: 32, 34, 36, 38, 40, and/or 42. In some embodiments, the one or more reverse primers comprise an antigenic tag. In certain embodiments, the concentration of the one or more reverse primers is at least 100 nM, at least 200 nM, at least 300 nM, at least 400 nM, at least 500 nM, at least 600 nM, at least 700 nM, at least 800 nM, at least 900 nM, or at least 1000 nM. In certain embodiments, the concentration of the one or more reverse primers is in a range from 100 nM to 200 nM, 100 nM to 500 nM, 100 nM to 800 nM, 100 nM to 1000 nM, 200 nM to 500 nM, 200 nM to 800 nM, 200 nM to 1000 nM, 500 nM to 800 nM, 500 nM to 1000 nM, or 800 nM to 1000 nM.

In some embodiments, the RPA reagents further comprises a probe. In certain embodiments, the probe is at least 70%, at least 75%, at least 80%, at least 85%, at least 90%, at least 95%, at least 99%, or 100% identical to SEQ ID NO: 28. In some embodiments, the probe is at least 70%, at least 75%, at least 80%, at least 85%, at least 90%, at least 95%, at least 99%, or 100% identical to SEQ ID Nos: 43-46. In some embodiments, the concentration of the probe is at least 50 nM, at least 60 nM, at least 70 nM, at least 80 nM, at least 90 nM, at least 100 nM, at least 110 nM, at least 120 nM, at least 130 nM, at least 140 nM, at least 150 nM, at least 160 nM, at least 170 nM, at least 180 nM, at least 190 nM, or at least 200 nM. In some embodiments, the concentration of the probe is in a range from 50 nM to 100 nM, 50 nM to 120 nM, 50 nM to 150 nM, 50 nM to 180 nM, 50 nM to 200 nM, 100 nM to 120 nM, 100 nM to 150 nM, 100 nM to 180 nM, 100 nM to 200 nM, 120 nM to 180 nM, 120 nM to 200 nM, or 150 nM to 200 nM.

In one embodiment, the RPA primers comprise SEQ ID NOs: 26-28; SEQ ID NOs: 39, 40, and 43; SEQ ID NOs: 39, 40, and 44; SEQ ID NOs. 41, 42, and 45; or SEQ ID NOs. 41, 42, and 46.

In some embodiments, the RPA reagents comprise RPA primers designed to amplify a human or animal nucleic acid that is not associated with a pathogen, a cancer cell, or a contaminant. In some such embodiments, the human or animal nucleic acid may act as a control. For example, successful amplification and detection of the control nucleic acid may indicate that the diagnostic test was properly run (e.g., sample was collected, cells were lysed, nucleic acids were amplified). On the other hand, failure to detect the control nucleic acid may indicate one or more of the following: improper specimen collection resulting in the lack of sufficient human sample material, improper extraction/purification of nucleic acid from the sample, ineffective inhibition of RNAse in the sample, improper assay set up and execution, and/or reagent or equipment malfunction.

In some instances, the control nucleic acid is a nucleic acid sequence encoding human RNase P. In some embodiments, the RPA reagents comprise primers (e.g., forward primers, reverse primers) and probes configured to detect a nucleic acid sequence encoding human RNase P.

In some embodiments, the RPA reagents comprise one or more recombinase enzymes. Non-limiting examples of suitable recombinase enzymes include T4 UvsX protein and T4 UvsY protein. In some embodiments, the concentration of each recombinase enzyme is at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, at least 0.10 mg/mL, at least 0.11 mg/mL, at least 0.12 mg/mL, at least 0.13 mg/mL, at least 0.14 mg/mL, or at least 0.15 mg/mL. In some embodiments, the concentration of each recombinase enzyme is in a range from 0.01 mg/mL to 0.05 mg/mL, 0.01 mg/mL to 0.1 mg/mL, 0.01 mg/mL to 0.15 mg/mL, 0.05 mg/mL to 0.1 mg/mL, 0.05 mg/mL to 0.15 mg/mL, or 0.10 mg/mL to 0.15 mg/mL.

In some embodiments, the RPA reagents comprise one or more single-stranded DNA binding proteins. A non-limiting example of a suitable single-stranded DNA binding protein is T4 gp32 protein. In certain embodiments, the concentration of the single-stranded DNA binding protein is at least 0.1 mg/mL, at least 0.2 mg/mL, at least 0.3 mg/mL, at least 0.4 mg/mL, at least 0.5 mg/mL, at least 0.6 mg/mL, at least 0.7 mg/mL, at least 0.8 mg/mL, at least 0.9 mg/mL, or at least 1.0 mg/mL. In certain embodiments, the concentration of the single-stranded DNA binding protein is in a range from 0.1 mg/mL to 0.2 mg/mL, 0.1 mg/mL to 0.5 mg/mL, 0.1 mg/mL to 0.8 mg/mL, 0.1 mg/mL to 1.0 mg/mL, 0.2 mg/mL to 0.5 mg/mL, 0.2 mg/mL to 0.8 mg/mL, 0.2 mg/mL to 1.0 mg/mL, 0.5 mg/mL to 0.8 mg/mL, 0.5 mg/mL to 1.0 mg/mL, or 0.8 mg/mL to 1.0 mg/mL.

In some embodiments, the RPA agents comprise a DNA polymerase. A non-limiting example of a suitable DNA polymerase is Staphylococcus aureus DNA polymerase (Sau). In certain embodiments, the concentration of the DNA polymerase is at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, or at least 0.1 mg/mL. In certain embodiments, the concentration of the single-stranded DNA binding protein is in a range from 0.01 mg/mL to 0.02 mg/mL, 0.01 mg/mL to 0.05 mg/mL, 0.01 mg/mL to 0.08 mg/mL, 0.01 mg/mL to 0.1 mg/mL, 0.02 mg/mL to 0.05 mg/mL, 0.02 mg/mL to 0.08 mg/mL, 0.02 mg/mL to 0.1 mg/mL, 0.05 mg/mL to 0.08 mg/mL, 0.05 mg/mL to 0.1 mg/mL, or 0.08 mg/mL to 0.1 mg/mL.

In some embodiments, the RPA agents comprise an endonuclease. A non-limiting example of a suitable endonuclease is Endonuclease IV. In some embodiments, the concentration of the endonuclease is at least 0.001 mg/mL, at least 0.002 mg/mL, at least 0.003 mg/mL, at least 0.004 mg/mL, at least 0.005 mg/mL, at least 0.006 mg/mL, at least 0.007 mg/mL, at least 0.008 mg/mL, at least 0.009 mg/mL, at least 0.01 mg/mL, at least 0.02 mg/mL, or at least 0.05 mg/mL. In some embodiments, the concentration of the endonuclease is in a range from 0.001 mg/mL to 0.005 mg/mL, 0.001 mg/mL to 0.01 mg/mL, 0.001 mg/mL to 0.02 mg/mL, 0.001 mg/mL to 0.05 mg/mL, 0.005 mg/mL to 0.01 mg/mL, 0.005 mg/mL to 0.02 mg/mL, 0.005 mg/mL to 0.05 mg/mL, 0.01 mg/mL to 0.02 mg/mL, or 0.01 mg/mL to 0.05 mg/mL.

In some embodiments, the RPA reagents comprise dNTPs (e.g., dATP, dGTP, dCTP, dTTP). In certain embodiments, the concentration of each dNTP is at least 0.1 mM, at least 0.2 mM, at least 0.3 mM, at least 0.4 mM, at least 0.5 mM, at least 0.6 mM, at least 0.7 mM, at least 0.8 mM, at least 0.9 mM, at least 1.0 mM, at least 1.1 mM, at least 1.2 mM, at least 1.3 mM, at least 1.4 mM, at least 1.5 mM, at least 1.6 mM, at least 1.7 mM, at least 1.8 mM, at least 1.9 mM, or at least 2.0 mM. In some embodiments, the concentration of each dNTP is in a range from 0.1 mM to 0.2 mM, 0.1 mM to 0.5 mM, 0.1 mM to 0.8 mM, 0.1 mM to 1.0 mM, 0.1 mM to 1.5 mM, 0.1 mM to 2.0 mM, 0.2 mM to 0.5 mM, 0.2 mM to 0.8 mM, 0.2 mM to 1.0 mM, 0.2 mM to 1.5 mM, 0.2 mM to 2.0 mM, 0.5 mM to 1.0 mM, 0.5 mM to 1.5 mM, 0.5 mM to 2.0 mM, 1.0 mM to 1.5 mM, 1.0 mM to 2.0 mM, or 1.5 mM to 2.0 mM.

In some embodiments, the RPA reagents comprise one or more additional components. Non-limiting examples of suitable components include DL-Dithiothreitol, phosphocreatine disodium hydrate, creatine kinase, and adenosine 5′-triphosphate disodium salt.

In some embodiments, a modified RPA protocol is used. For example, RPA may be performed using small labels (e.g., labeled haptens) for the undiluted (direct) detection of RPA products on lateral flow strips (Powell et al., Analytical Biochem., 2018, 543(15): 108-115). In some cases, RPA reactions present as a phase-separated system in which the core RPA proteins are found in colloidal globules, which may result in the sequestration of signals (e.g., biotin) and poor signaling results. To avoid this, smaller analytes (e.g., haptens) may be employed in conjunction with formamidopyrimidine DNA glycosylase (Fpg). In some embodiments, dual hapten probes are used. In some embodiments, the dual hapten probes comprise two haptens joined by a linker, such as a lysine residue. The smaller probes (e.g., dual hapten probes) may be separated from the reaction until after amplification has occurred. Then, an Fpg probe against a specific amplicon target conjugated to the dual-hapten label may be released. The probe may bind to the specific amplicon target, and Fpg may cleave the dual-hapten label. Since the dual-hapten label is relatively small, it may be able to readily leave the RPA globules. Then, the label may be detected by any means known in the art, such as with a sandwich immunoassay on a lateral flow strip (e.g., a lateral flow test comprising an antibody against one of the analytes and gold particles comprising antibodies against the second analyte). Non-limiting examples of dual haptens for labeling are shown in FIG. 4 and include biotin in combination with digoxigenin, FITC, Texas Red, dinitrophenol (DNP), FLAG peptide (DYKDDDDK; SEQ ID NO: 22), His peptide (HHHHHH; SEQ ID NO: 23), HA peptide (YPYDVPDYA; SEQ ID NO: 24), and Myc peptide (EQKLISEEDL; SEQ ID NO: 25).

Nicking Enzyme Amplification Reaction (NEAR)

In some embodiments, amplification of one or more target nucleic acids is accomplished through the use of a nicking enzyme amplification reaction (NEAR) reaction. Accordingly, in some embodiments the nucleic acid amplification reagents are NEAR reagents. NEAR generally refers to a method for amplifying a target nucleic acid using a nicking endonuclease and a strand displacing DNA polymerase. In some cases, NEAR may allow for amplification of very small amplicons.

In some embodiments, the NEAR reagents comprise a forward template and a reverse template. In certain embodiments, the forward template comprises a nucleic acid sequence having a hybridization region at the 3′ end that is complementary to the 3′ end of a target antisense strand (e.g., an antisense sequence to the reverse-transcribed SARS-CoV-2 nucleocapsid sequence), a nicking enzyme binding site and a nicking site upstream of the hybridization region, and a stabilizing region upstream of the nicking site. In certain embodiments, the first reverse template comprises a nucleic acid sequence having a hybridization region at the 3′ end that is complementary to the 3′ end of a target gene sense strand (e.g., a SARS-CoV-2 nucleocapsid gene sense strand), a nicking enzyme binding site and a nicking site upstream of the hybridization region, and a stabilizing region upstream of the nicking site. Designs of templates suitable for NEAR methods disclosed herein are provided in, for example, U.S. Pat. Nos. 9,617,586 and 9,689,031, each of which are incorporated herein by reference.

In some embodiments, the NEAR composition further comprises a probe oligonucleotide. In certain embodiments, the probe comprises a nucleotide sequence complementary to the target gene nucleotide sequence. In some instances, for example, the probe is a SARS-CoV-2 specific probe.

In some embodiments, the probe is conjugated to a detectable label. In some embodiments, the detectable label is selected from the group consisting of a fluorophore, an enzyme, a quencher, an enzyme inhibitor, a radioactive label, a member of a binding pair, and a combination thereof. In some embodiments, one or more of the forward template and the reverse template comprises a least one modified nucleotide, spacer, or blocking group. In some embodiments, at least one modified nucleotide includes a 2′ modification.

In some embodiments, the NEAR reagents comprise a DNA polymerase. Examples of suitable DNA polymerases include, but are not limited to, Geobacillus bogazici DNA polymerase, Bst (large fragment), exo-DNA Polymerase, and Manta 1.0 DNA Polymerase (Enzymatics 3 e). In some embodiments, the NEAR reagents comprise at least one nicking enzyme. Non-limiting examples of suitable nicking enzymes include Nt. BspQI, Nb. BbvCi, Nb. BsmI, Nb. BsrDI, Nb. BtsI, Nt. AlwI, Nt. BbvCI, Nt. BstNBI, Nt. CviPII, Nb. Bpul OI, Nt. BpulOI, and N. BspD61. In some embodiments, the NEAR reagents further comprise dNTPs (e.g., dATP, dGTP, dCTP, dTTP).

Amplification Heating Protocol

In some embodiments, an isothermal amplification method described herein comprises applying heat to a sample according to an amplification heating protocol. In certain instances, an amplification method comprises applying an amplification heating protocol comprising heating the sample at one or more temperatures for one or more time periods using any heater described herein. However, other embodiments of the present invention do not require a step of applying heat to a sample. In such embodiments, the step of applying an amplification heating protocol as described below would not be necessary for nucleic acid amplification, and would not be performed.

In some embodiments, an amplification heating protocol comprises heating the sample at a first temperature for a first time period. In certain instances, the first temperature is at least 30° C., at least 32° C., at least 37° C., at least 50° C., at least 60° C., at least 63.5° C., at least 65° C., at least 70° C., at least 80° C., or at least 90° C. In some embodiments, the first temperature is 37° C. In certain instances, the first temperature is in a range from 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the first time period is at least 1 minute, at least 2 minutes, at least 3 minutes, at least 4 minutes, at least 5 minutes, at least 10 minutes, at least 15 minutes, at least 20 minutes, at least 30 minutes at least 40 minutes, at least 50 minutes, or at least 60 minutes. In some embodiments, the first time period is 3 minutes. In certain instances, the first time period is in a range from 1 to 3 minutes, 1 to 5 minutes, 1 to 10 minutes, 1 to 15 minutes, 1 to 20 minutes, 1 to 30 minutes, 1 to 40 minutes, 1 to 50 minutes, 1 to 60 minutes, 3 to 5 minutes, 3 to 10 minutes, 3 to 15 minutes, 3 to 20 minutes, 3 to 30 minutes, 3 to 40 minutes, 3 to 50 minutes, 3 to 60 minutes, 5 to 10 minutes, 5 to 15 minutes, 5 to 20 minutes, 5 to 30 minutes, 5 to 40 minutes, 5 to 50 minutes, 5 to 60 minutes, 10 to 20 minutes, 10 to 30 minutes, 10 to 40 minutes, 10 to 50 minutes, 10 to 60 minutes, 20 to 30 minutes, 20 to 40 minutes, 20 to 50 minutes, 20 to 60 minutes, 30 to 40 minutes, 30 to 50 minutes, 30 to 60 minutes, 40 minutes to 50 minutes, 40 minutes to 60 minutes, or 50 minutes to 60 minutes.

In some embodiments, an amplification heating protocol comprises heating the sample at a second temperature for a second time period. In certain instances, the second temperature is at least 30° C., at least 32° C., at least 37° C., at least 50° C., at least 60° C., at least 63.5° C., at least 65° C., at least 70° C., at least 80° C., or at least 90° C. In some embodiments, the second temperature is 63.5° C. In certain instances, the second temperature is in a range from 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the second time period is at least 1 minute, at least 2 minutes, at least 3 minutes, at least 4 minutes, at least 5 minutes, at least 10 minutes, at least 15 minutes, at least 20 minutes, at least 30 minutes, at least 40 minutes, at least 45 minutes, at least 50 minutes, or at least 60 minutes. In some embodiments, the second time period is 40 minutes. In certain instances, the second time period is in a range from 1 to 3 minutes, 1 to 5 minutes, 1 to 10 minutes, 1 to 15 minutes, 1 to 20 minutes, 1 to 30 minutes, 1 to 45 minutes, 1 to 50 minutes, 1 to 60 minutes, 3 to 5 minutes, 3 to 10 minutes, 3 to 15 minutes, 3 to 20 minutes, 3 to 30 minutes, 3 to 45 minutes, 3 to 50 minutes, 3 to 60 minutes, 5 to 10 minutes, 5 to 15 minutes, 5 to 20 minutes, 5 to 30 minutes, 5 to 45 minutes, 5 to 50 minutes, 5 to 60 minutes, 10 to 20 minutes, 10 to 30 minutes, 10 to 45 minutes, 10 to 50 minutes, 10 to 60 minutes, 20 to 30 minutes, 20 to 45 minutes, 20 to 50 minutes, 20 to 60 minutes, 30 to 45 minutes, 30 to 50 minutes, 30 to 60 minutes, or 45 to 60 minutes. In some embodiments, an amplification heating protocol does not comprise a second time period for heating.

In some embodiments, an amplification heating protocol comprises heating the sample at a third temperature for a third time period. In certain instances, the third temperature is at least 30° C., at least 32° C., at least 37° C., at least 50° C., at least 60° C., at least 63.5° C., at least 65° C., at least 70° C., at least 80° C., or at least 90° C. In some embodiments, the third temperature is 37° C. In certain instances, the third temperature is in a range from 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the third time period is at least 1 minute, at least 2 minutes, at least 3 minutes, at least 4 minutes, at least 5 minutes, at least 10 minutes, at least 15 minutes, at least 20 minutes, at least 30 minutes, at least 45 minutes, or at least 60 minutes. In certain instances, the third time period is in a range from 1 to 3 minutes, 1 to 5 minutes, 1 to 10 minutes, 1 to 15 minutes, 1 to 20 minutes, 1 to 30 minutes, 1 to 45 minutes, 1 to 60 minutes, 3 to 5 minutes, 3 to 10 minutes, 3 to 15 minutes, 3 to 20 minutes, 3 to 30 minutes, 3 to 45 minutes, 3 to 60 minutes, 5 to 10 minutes, 5 to 15 minutes, 5 to 20 minutes, 5 to 30 minutes, 5 to 45 minutes, 5 to 60 minutes, 10 to 20 minutes, 10 to 30 minutes, 10 to 45 minutes, 10 to 60 minutes, 20 to 30 minutes, 20 to 45 minutes, 20 to 60 minutes, 30 to 45 minutes, 30 to 60 minutes, or 45 to 60 minutes. In some embodiments, an amplification heating protocol does not comprise a third time period for heating.

In some embodiments, an amplification heating protocol may comprise heating a sample at one or more additional temperatures for one or more additional time periods.

### Lyophilized Reagents

In some cases, one or more reagents described herein (e.g., lysis reagents, nucleic acid amplification reagents, reagents for reducing or eliminating cross contamination) are in solid form (e.g., lyophilized, dried, crystallized, air jetted). In certain cases, one or more (and, in some cases, all) nucleic acid amplification reagents are in solid form. In some cases, one or more (and, in some cases, all) lysis reagents are in solid form. In certain embodiments, all reagents of a diagnostic test, system, or method are in solid form. In some embodiments, the one or more reagents in solid form are in the form of one or more beads and/or tablets. The one or more beads and/or tablets may comprise any reagent or combination of reagents described herein.

In some embodiments, the one or more beads and/or tablets are stable at room temperature for a relatively long period of time. In certain embodiments, the one or more beads and/or tablets are stable at room temperature for at least 1 month, at least 3 months, at least 6 months, at least 9 months, at least 1 year, at least 2 years, at least 3 years, at least 4 years, at least 5 years, at least 6 years, at least 7 years, at least 8 years, at least 9 years, or at least 10 years. In some embodiments, the one or more beads and/or tablets are stable at room temperature for 1-3 months, 1-6 months, 1-9 months, 1 month to 1 year, 1 month to 2 years, 1 month to 5 years, 1 month to 10 years, 3-6 months, 3-9 months, 3 months to 1 year, 3 months to 2 years, 3 months to 5 years, 3 months to 10 years, 6-9 months, 6 months to 1 year, 6 months to 2 years, 6 months to 5 years, 6 months to 10 years, 9 months to 1 year, 9 months to 2 years, 9 months to 5 years, 9 months to 10 years, 1-2 years, 1-3 years, 1-4 years, 1-5 years, 1-6 years, 1-7 years, 1-8 years, 1-9 years, 1-10 years, 2-5 years, 2-10 years, 3-5 years, 3-10 years, 4-10 years, 5-10 years, 6-10 years, 7-10 years, 8-10 years, or 9-10 years.

In some embodiments, the one or more beads and/or tablets are thermostabilized and is stable across a wide range of temperatures. In some embodiments, the one or more beads and/or tablets are stable at a temperature of at least 0° C., at least 10° C., at least 20° C., at least 25° C., at least 30° C., at least 37° C., at least 40° C., at least 50° C., at least 60° C., at least 65° C., at least 70° C., at least 80° C., at least 90° C., or at least 100° C. In some embodiments, the one or more beads and/or tablets are stable at a temperature in a range from 0° C. to 10° C., 0° C. to 20° C., 0° C. to 25° C., 0° C. to 30° C., 0° C. to 37° C., 0° C. to 40° C., 0° C. to 50° C., 0° C. to 60° C., 0° C. to 65° C., 0° C. to 70° C., 0° C. to 80° C., 0° C. to 90° C., 0° C. to 100° C., 10° C. to 20° C., 10° C. to 25° C., 10° C. to 30° C., 10° C. to 37° C., 10° C. to 40° C., 10° C. to 50° C., 10° C. to 60° C., 10° C. to 65° C., 10° C. to 70° C., 10° C. to 80° C., 10° C. to 90° C., 10° C. to 100° C., 20° C. to 25° C., 20° C. to 30° C., 20° C. to 37° C., 20° C. to 40° C., 20° C. to 50° C., 20° C. to 60° C., 20° C. to 65° C., 20° C. to 70° C., 20° C. to 80° C., 20° C. to 90° C., 20° C. to 100° C., 25° C. to 30° C., 25° C. to 37° C., 25° C. to 40° C., 25° C. to 50° C., 25° C. to 60° C., 25° C. to 65° C., 25° C. to 70° C., 25° C. to 80° C., 25° C. to 90° C., 25° C. to 100° C., 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C.

Lyophilized Amplification Pellets

In some embodiments, the amplification step comprises contacting the sample to be amplified with a lyophilized amplification pellet. The lyophilized amplification pellet may comprise one or more (and, in some cases, all) of the nucleic acid amplification reagents for a nucleic acid amplification reaction. In some embodiments, the lyophilized amplification pellet comprises one or more of the following components: Reverse Transcriptase, murine RNAse inhibitor, T4 UvsX Protein, T4 UvsY Protein, T4 gp32 Protein, Endonuclease IV, Staphylococcus aureus DNA polymerase (Sau), Test Primer1 Fwd, Test Primer1 Rev Test Probe1 Control Primer1 Fwd, Control Primer1 Rev, Control Probe1, DL-Dithiothreitol, Phosphocreatine disodium hydrate, Creatine Kinase, Adenosine 5′-triphosphate disodium salt, Tris(hydroxymethyl)aminomethane (Tris), Deoxy-nucleotide triphosphates (dATP:dCTP:dGTP:dTTP), and Deoxyuridine triphosphate Solution (dU). In some embodiments, the lyophilized pellet comprises Reverse Transcriptase, murine RNAse inhibitor, T4 UvsX Protein, T4 UvsY Protein, T4 gp32 Protein, Endonuclease IV, Staphylococcus aureus DNA polymerase (Sau), Test Primer1 Fwd, Test Primer1 Rev Test Probe1 Control Primer1 Fwd, Control Primer1 Rev, Control Probe1, DL-Dithiothreitol, Phosphocreatine disodium hydrate, Creatine Kinase, Adenosine 5′-triphosphate disodium salt, Tris(hydroxymethyl)aminomethane (Tris), Deoxy-nucleotide triphosphates (dATP:dCTP:dGTP:dTTP), and Deoxyuridine triphosphate Solution (dU).

As an illustrative, non-limiting example, a lyophilized amplification pellet may comprise the following components:

| Component | Target Concentration | |
|---|---|---|
| Reverse Transcriptase | 10 | U/μL |
| murine RNAse inhibitor | 1 | U/μL |
| T4 UvsX Protein | 0.12 | mg/mL |
| T4 UvsY Protein | 0.06 | mg/mL |
| T4 gp32 Protein | 0.6 | mg/mL |
| Endonuclease IV | 0.0046 | mg/mL |
| Staphylococcus aureus DNA polymerase | 0.0128 | mg/mL |
| (Sau) | | |
| Test Primer 1 Fwd | 420 | nM |
| Test Primer 1 Rev | 420 | nM |
| Test Probe 1 | 120 | nM |
| Control Primer 1 Fwd | 420 | nM |
| Control Primer 1 Rev | 420 | nM |
| Control Probe 1 | 120 | nM |
| DL-Dithiothreitol | 2 | mM |
| Phosphocreatine disodium hydrate | 50 | mM |
| Creatine Kinase | 0.1 | mg/mL |
| Adenosine 5′-triphosphate disodium salt | 3 | mM |
| Tris(hydroxymethyl)aminomethane (Tris) | 50 | mM |
| Deoxy-nucleotide triphosphates | 0.2 | mM each |
| (dATP:dCTP:dGTP:dTTP) | | |
| Deoxyuridine triphosphate Solution (dU) | 0.2 | mM |

Lyophilized Lysis Pellets

In some embodiments, the lysis step comprises contacting the sample to be amplified with a lyophilized lysis pellet. The lyophilized lysis pellet may comprise one or more (and, in some cases, all) of the lysis reagents required for lysis of cells within a sample. In some embodiments, the lyophilized lysis pellet comprises Thermolabile Uracil-DNA Glycosylase (UDG). In some embodiments, the lyophilized lysis pellet comprises a murine RNase inhibitor. In one non-limiting embodiment, the lyophilized lysis pellet comprises 0.02 U/μL of UDG and 1 U/μL of murine RNase inhibitor.

### Additional Reagents

In some embodiments, one or more reagents of a diagnostic system further comprise one or more additives that may enhance reagent stability (e.g., protein stability). Non-limiting examples of suitable additives include trehalose, polyethylene glycol (PEG), polyvinyl alcohol (PVA), and glycerol.

### Molecular Switches

As described herein, a sample may undergo lysis and amplification prior to detection. The reagents associated with lysis and/or detection may be in solid form (e.g., lyophilized, dried, crystallized, air jetted, etc.). In certain embodiments, one or more (and, in some cases, all) of the reagents necessary for lysis and/or amplification may be present in a single bead, pellet, capsule, gelcap, or tablet. In some embodiments, the bead, pellet, capsule, gelcap, or tablet may comprise two or more enzymes, and it may be necessary for the enzymes to be activated in a particular order. Therefore, in some embodiments of the present technology, the enzyme-containing bead, pellet, capsule, gelcap, or tablet may further comprise one or more molecular switches. Molecular switches, as used or described herein, may be molecules that, in response to certain conditions, reversibly switch between two or more stable states. In some embodiments, the condition that causes the molecular switch to change its configuration may be associated with any one or any combination of: pH, light, temperature, an electric current, microenvironment, and the presence of ions and/or other ligands. In one embodiment, the condition may be heat. In some embodiments, the molecular switches described herein may be aptamers. Aptamers generally refer to oligonucleotides or peptides that bind to specific target molecules (e.g., the enzymes described herein). The aptamers, upon exposure to heat or other conditions, may dissociate from the enzymes. With the use of molecular switches, the processes described herein (e.g., lysis, decontamination, reverse transcription, and amplification, etc.) may be performed in a single test tube with a single enzymatic tablet, pellet, capsule, or gelcap.

In one illustrative embodiment, an enzymatic bead, pellet, capsule, gelcap, or tablet may comprise UDG, reverse transcriptase, and DNA polymerase (e.g., Bst DNA polymerase). Initially, the sample may be heated at 37° C., which may be a temperature at which UDG is active, in order to decontaminate the sample. At 37° C., molecular switches may bind to, and inactivate, the reverse transcriptase and DNA polymerase. This may advantageously ensure that they do not interfere with the UDG decontamination reaction. Next, following decontamination, the sample may be heated at 65° C., which may deactivate heat-sensitive UDG but may cause the molecular switches to release, and therefore activate, the reverse transcriptase and DNA polymerase. Reverse transcription may then proceed.

Therefore, in some embodiments, the molecular switches (aptamers) may specifically bind the enzymes described herein, such that the enzymes are inactivated. The term “inactivated,” as used herein, may refer to or be used to describe an enzyme that is not enzymatically active; that is, it cannot perform its enzymatic function. Aptamers, as described herein, may be single-stranded nucleic acid molecules (about 5-25 kDa) having unique configurations that may allow them to bind to molecular targets with high specificity and affinity. In one embodiment, the aptamers may be DNA or RNA aptamers or hybrid DNA/RNA aptamers. Similar to antibodies, aptamers may possess binding affinities in the low nanomolar to picomolar range.

The small size of an aptamer may enhance its ability to bind to a specific site on an enzyme, thus enabling the aptamer to alter the function of that site without affecting the functions of other sites on the enzyme. In some embodiments of the present technology, the aptamers may inhibit the enzymatic activity of a reverse transcriptase, a DNA polymerase (e.g., Bst DNA polymerase), and/or a glycosylase. In some embodiments, the presently disclosed methods may produce at least about a 10%, 15%, 20%, 25%, 30%, 35%, 40%, 45%, 50%, 55%, 60%, 65%, 70%, 75%, 80%, 85%, 90%, 95%, or even 100% inhibition of enzymatic activity relative to enzymatic activity measured in absence of aptamers (e.g., a control) in an assay.

The term “specifically binds,” as used herein, may refers to a molecule (e.g., aptamer) that binds to a target (e.g., an enzyme) with at least five-fold greater affinity as compared to any non-targets, e.g., at least 10-, 20-. 50-, or 100-fold greater affinity,

The length of the aptamers is not limited, but typical aptamers may have a length of about 10 to about 120 nucleotides, such as about 15 nucleotides, about 20 nucleotides, about 25 nucleotides, about 30 nucleotides, about 35 nucleotides, about 40 nucleotides, about 45 nucleotides, about 50 nucleotides, about 55 nucleotides, about 60 nucleotides, about 65 nucleotides, about 70 nucleotides, about 75 nucleotides, about 80 nucleotides, about 85 nucleotides, about 90 nucleotides, about 95 nucleotides, about 100 nucleotides, about 105 nucleotides, about 110 nucleotides, about 115 nucleotides, about 120 nucleotides, or more nucleotides. In certain embodiments, the aptamer may have additional nucleotides attached to the 5′- and/or 3′ end,

The polynucleotide aptamers may be comprised of ribonucleotides only (RNA aptamers), deoxyribonucleotides only (DNA aptamers), or a combination of ribonucleotides and deoxyribonucleotides. The nucleotides may be naturally occurring nucleotides (e.g., ATP, TTP, GTP, CTP, UTP) or modified nucleotides. As used herein, the term “modified nucleotide” may refer to a nucleotide comprising a base such as, for example, adenine, guanine, cytosine, thymine, and uracil, xanthine, inosine, and queuosine that may have been modified by the replacement or addition of one or more atoms or groups. For example, the modification may comprise a nucleotide that is modified with respect to the base moiety, such as a/an alkylated, halogenated, thiolated, aminated, amidated, or acetylated base, in various combinations. Modified nucleotides also may include nucleotides that comprise a sugar moiety modification (e.g., 2′-fluoro or 2′-O-methyl nucleotides), as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles.

### Nucleic Acid Detection and Readout

Following cell lysis, nucleic acid extraction/purification (if applicable), and nucleic acid amplification, aspects of the invention include a step of detection, wherein target nucleic acids are detected within the amplified nucleic acid population of the sample. In some embodiments, targeted subsets of the amplified nucleic acids (i.e., “amplicons”) may be detected using any suitable methods, including, but not limited to, those described herein.

In some embodiments, one or more target nucleic acid sequences are detected using a lateral flow assay strip. In some embodiments, one or more target nucleic acid sequences are detected using a colorimetric assay. In some embodiments, one or more target nucleic acid sequences are detected using CRISPR/Cas-mediated detection.

Such detection of target and/or control nucleic acid sequences may in some embodiments by visualized directly by the user, for example, as an opaque line on a lateral flow assay, as a change in color in a colorimetric assay, or by any other detectable moiety able to be visualized as described herein. Accordingly, the readout of the rapid diagnostic test may in some embodiments consist of direct visualization by the user of the test results. As used herein, “readout” refers to the communication of the rapid diagnostic test detection results to a user of the rapid diagnostic test, system, or kit.

Additionally or alternatively, in some embodiments use of a rapid diagnostic test of the present invention is guided by a downloadable software application which detects the presence of target nucleic acid(s). In such embodiments, the readout of the rapid diagnostic test may be presented by the software application to the user. Additionally or alternatively, in some embodiments the readout of the rapid diagnostic test is integrated into a software-based testing ecosystem.

### Lateral Flow Test

In some embodiments, one or more target nucleic acid sequences are detected using a lateral flow test or lateral flow assay strip (e.g., in a “chimney” readout device). As used herein, lateral flow “test” or “test strip” and lateral flow “assay strip” are used interchangeably and refer to a device intended to detect the presence of a target substance in a sample by moving the sample along a surface comprising reactive molecules that indicate the presence of the target substance. A lateral flow test may comprise a lateral flow assay strip.

Generally, lateral flow tests comprise an assay strip comprising, in order of flow direction, a sample region and a results region. The sample region and/or results region may each comprise multiple sub-regions. For example, the sample region may comprise first and second sample sub-regions, and the results region may comprise first and second results sub-regions. As will be understood by reference to the accompanying drawings, the first and second sample sub-regions may also be considered the first and second sub-regions of the lateral flow assay strip. Likewise, the first and second results sub-regions may also be considered the third and fourth sub-regions of the lateral flow assay strip.

A processed sample (e.g., a sample which has undergone the steps of cell lysis, nucleic acid extraction/purification (if applicable), and nucleic acid amplification) is added to the sample region. The results region comprises at least one test line and at least one control line. The test and/or control lines comprise a probe, for example, an antibody, that recognizes a specific nucleic acid sequence. For example, a test line probe will recognize the target nucleic acid sequence. If the sample comprises the target nucleic acid sequence, the sample will interact with the test line, and the target nucleic acid sequence is detectable. If the sample does not comprise the target nucleic acid sequence, the sample will not interact with the test line, and the target nucleic acid sequence is not detectable by virtue of its absence. Typically, a positive result (e.g., detection of the presence of the target nucleic acid in the sample) is visualized to a user via the opaque marking of a test line which can be readily observed by the user. A negative result (e.g., no detection of the target nucleic acid in the sample) is visualized by the lack of darkening of the test line.

As described elsewhere herein, a sample may in some embodiments be combined with reagents, buffers, or other types of fluid in order to lyse, amplify, or otherwise process the sample material prior to the step of detection. Such a processed sample may in some embodiments be referred to as a “fluidic sample” or a “processed sample.” In some embodiments, a fluidic sample comprises amplicons. In some embodiments, a fluidic sample (e.g., fluidic contents of a reaction tube comprising the sample) is transported through the lateral flow assay strip via capillary flow. The lateral flow test relies on capillary flow of the amplified sample through a membrane and across discrete strips of capture antibodies. If the sample does not wick completely through the strip, then the assay is invalid.

In certain cases, the lateral flow assay strip may comprise one or more fluid-transporting layers comprising one or more absorbent materials that allow fluid transport (e.g., via capillary action). Non-limiting examples of suitable materials may include polyethersulfone, cellulose, polycarbonate, nitrocellulose, sintered polyethylene, and glass fibers. It should be understood that the word “flow” in the term “lateral flow assay strip” indicates movement, which may occur by wicking movement or capillary-action movement, and which need not require flowing movement of any liquid, although flowing movement may occur.

In some embodiments of the present technology, the one or more fluid-transporting layers of the lateral flow assay strip may comprise a plurality of fibers (e.g., woven or non-woven fabrics). In some embodiments, the one or more fluid-transporting layers may comprise a plurality of pores. In some embodiments, pores and/or interstices between fibers may advantageously facilitate fluid transport (e.g., via capillary action). The pores may have any suitable average pore size. In certain embodiments, the plurality of pores may have an average pore size of 30 μm or less, 25 μm or less, 20 μm or less, 15 μm or less, 10 μm or less, 5 μm or less, 2 μm or less, 1 μm or less, 0.9 μm or less, 0.8 μm or less, 0.7 μm or less, 0.6 μm or less, 0.5 μm or less, 0.4 μm or less, 0.3 μm or less, 0.2 μm or less, or 0.1 μm or less. In certain embodiments, the plurality of pores may have an average pore size of at least 0.1 μm, at least 0.2 μm, at least 0.3 μm, at least 0.4 μm, at least 0.5 μm, at least 0.6 μm, at least 0.7 μm, at least 0.8 μm, at least 0.9 μm, at least 1 μm, at least 2 μm, at least 5 μm, at least 10 μm, at least 15 μm, at least 20 μm, at least 25 μm, or at least 30 μm. In some embodiments, the plurality of pores may have an average pore size in a range from 0.1 μm to 0.5 μm, 0.1 μm to 1 μm, 0.1 μm to 5 μm, 0.1 μm to 10 μm, 0.1 μm to 15 μm, 0.1 μm to 20 μm, 0.1 μm to 25 μm, 0.1 μm to 30 μm, 0.5 μm to 1 μm, 0.5 μm to 5 μm, 0.5 μm to 10 μm, 0.5 μm to 15 μm, 0.5 μm to 20 μm, 0.5 μm to 25 μm, 0.5 μm to 30 μm, 1 μm to 5 μm, 1 μm to 10 μm, 1 μm to 15 μm, 1 μm to 20 μm, 1 μm to 25 μm, 1 μm to 30 μm, 5 μm to 10 μm, 5 μm to 15 μm, 5 μm to 20 μm, 5 μm to 25 μm, 5 μm to 30 μm, 10 μm to 15 μm, 10 μm to 20 μm, 10 μm to 25 μm, 10 μm to 30 μm, 15 μm to 20 μm, 15 μm to 25 μm, 15 μm to 30 μm, or 20 μm to 30 μm.

The one or more fluid-transporting layers of the lateral flow assay strip may have any suitable porosity. In some embodiments of the present technology, the one or more fluid transporting layers may have a porosity of at least 10%, at least 20%, at least 30%, at least 40%, at least 50%, or at least 60%. In some embodiments, the one or more fluid-transporting layers may have a porosity in a range from 10% to 20%, 10% to 30%, 10% to 40%, 10% to 50%, 10% to 60%, 20% to 40%, 20% to 50%, 20% to 60%, 30% to 50%, 30% to 60%, 40% to 60%, or 50% to 60%.

Sample Region

In some embodiments, the fluidic sample is introduced to a sample region of the lateral flow test. In some embodiments, the sample region comprises two or more sample sub-regions (e.g., 2, 3, 4, or 5 sample sub-regions). In some embodiments, the sample region comprises two sample sub-regions. In some embodiments, the fluidic sample is introduced to a first sample sub-region (e.g., a sample pad) of the lateral flow assay strip.

In certain embodiments, the fluidic sample subsequently flows through a second sample sub-region (e.g., a particle conjugate pad) comprising a plurality of labeled particles. In some cases, the particles comprise gold nanoparticles (e.g., colloidal gold nanoparticles). The particles may be labeled with any suitable label. Non-limiting examples of suitable labels include biotin, streptavidin, fluorescein isothiocyanate (FITC), fluorescein amidite (FAM), fluorescein, and digoxigenin (DIG). In some cases, as an amplicon-containing fluidic sample flows through the second sub-region (e.g., a particle conjugate pad), a labeled nanoparticle binds to a label of an amplicon, thereby forming a particle-amplicon conjugate.

Results Region

In some embodiments, after being introduced to the sample region, the fluidic sample is subsequently introduced to a results region of the lateral flow test. The results region comprises at least one test line and at least one control line. The test line comprises a probe (e.g., an antibody) that recognizes a target nucleic acid sequence. The control line comprises a probe (e.g., an antibody) that recognizes a control nucleic acid sequence. In some embodiments, the results region comprises a single region. In some embodiments, the results region comprises two or more results sub-regions.

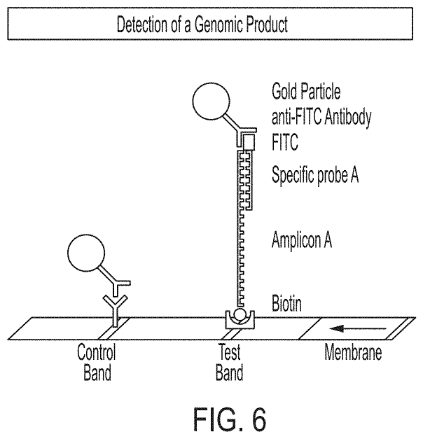

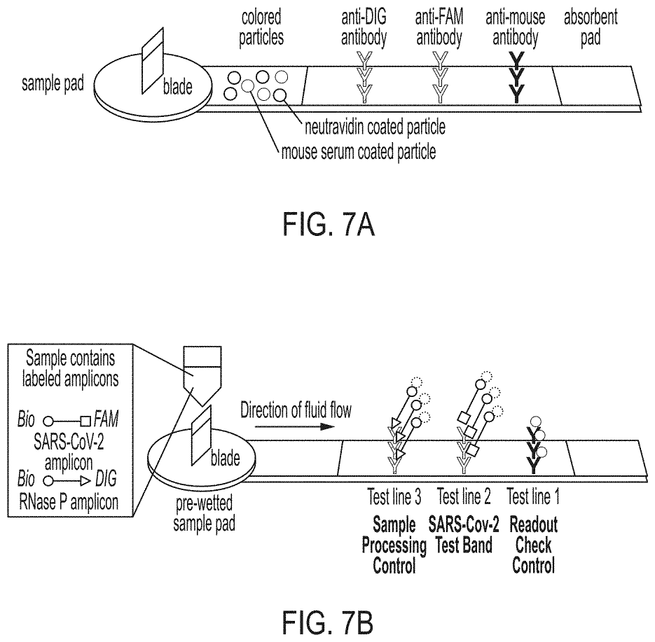

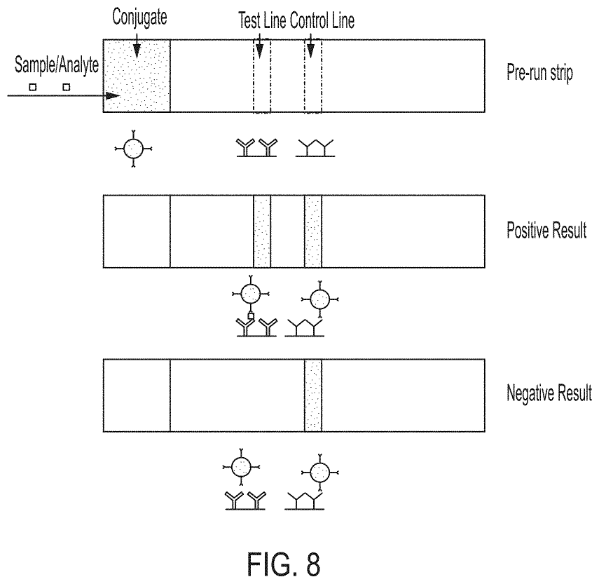

In some embodiments, the fluidic sample (e.g., comprising a particle-amplicon conjugate) subsequently flows through a first results sub-region (e.g., a test pad) comprising a test line. In some embodiments, the test line comprises a capture reagent (e.g., a probe, such as an immobilized antibody) configured to detect a target nucleic acid. In some embodiments, the test line comprises a capture reagent that will bind to a target nucleic acid sequence, and is detectable. In one embodiment, the test line is detectable using an anti-FITC antibody conjugated to a gold particle. In such embodiments, only one target nucleic acid will be detected. In some embodiments, a particle-amplicon conjugate may be captured by one or more capture reagents (e.g., immobilized antibodies), and an opaque marking may appear. The marking may have any suitable shape or pattern (e.g., one or more straight lines, curved lines, dots, squares, check marks, x marks). An exemplary illustration is shown in FIG. 6.

In some embodiments, the fluidic sample (e.g., comprising a particle-amplicon conjugate) subsequently flows through a first results sub-region (e.g., a test pad) comprising more than one test line. In some instances, each test line of the lateral flow assay strip is configured to detect a different target nucleic acid (e.g., multiplexed detection). In such embodiments, multiple target nucleic acids may be detected. Lateral flow assay strips for multiplexed testing are described in more detail elsewhere herein. In some instances, two or more test lines of the lateral flow assay strip are configured to detect the same target nucleic acid. The test line(s) may have any suitable shape or pattern (e.g., one or more straight lines, curved lines, dots, squares, check marks, x marks).

In some embodiments, the target nucleic acid sequence or sequences is one or more sequences from the processed sample (e.g., a coronavirus- and/or influenza-specific nucleic acid sequence). The target sequence or sequences may, in some embodiments, be the same sequence or sequences targeted by the amplification primers as described elsewhere herein (e.g., LAMP primers, RPA primers, NEAR primers, etc.). In some embodiments, the target sequence is specific for COVID-19. In some embodiments, the target sequence is specific for influenza type A. In some embodiments, the target sequence is specific for influenza type B.

In certain embodiments, the first results sub-region (e.g., the test pad) further comprises one or more control lines. In some embodiments, the control line comprises a capture reagent that will bind to a nucleic acid sequence, and is detectable. In one embodiment, the control line is detectable using an anti-FITC antibody conjugated to a gold particle. In some embodiments, a control nucleic acid may be captured by one or more capture reagents (e.g., immobilized antibodies), and an opaque marking may appear. The marking may have any suitable shape or pattern (e.g., one or more straight lines, curved lines, dots, squares, check marks, x marks).

In some embodiments, the control nucleic acid sequence or sequences is one or more sequences from the processed sample. In certain instances, the control line is a human (or animal) nucleic acid control line. In some embodiments, for example, the control line is configured to detect a control nucleic acid (e.g., RNase P) sequence that is generally present in all humans (or animals). In some embodiments, the control sequence or sequences may in some embodiments be the same sequence or sequences targeted by the amplification primers as described elsewhere herein (e.g., LAMP primers, RPA primers, NEAR primers, etc.). In some cases, the control line becoming detectable indicates that the sample was successfully collected, nucleic acids from the sample were amplified, and the amplicons were transported through the entire lateral flow assay strip. In some embodiments, the target sequence is specific for COVID-19.

In certain instances, the control line is a lateral flow control line. In some embodiments, the lateral flow control line is located at the very end of the assay strip. In some cases, the lateral flow control line becoming detectable indicates that a liquid was successfully transported through the lateral flow assay strip.

In some embodiments, the lateral flow assay strip comprises two or more control lines. In some embodiments, the lateral flow assay strip comprises two control lines. In some embodiments, the lateral flow assay strip comprises a human (or animal) nucleic acid control line and a lateral flow control line.

Failure to detect the control line on the lateral flow assay strip indicates failure of the sample to be properly delivered to the lateral flow assay strip, and an invalid result. The failure to detect a positive control may indicate one or more of the following: improper specimen collection resulting in the lack of sufficient sample material in the diagnostic assay, improper extraction of nucleic acids from clinical materials resulting in loss of nucleic acids and/or nucleic acid degradation, ineffective inhibition of RNAse in the patient sample resulting in RNA degradation, improper assay set up and execution, and/or reagent or equipment malfunction. In instances where the control line is not detected, the test is invalid.

Successful detection of the control line on the lateral flow assay strip indicates successful collection, extraction, RNase protection, and amplification of nucleic acids from the sample. Amplification of all samples is expected to result in the appearance of a visible band on the lateral flow strip at the positive control location. A positive result on the positive control band indicates that the user successfully obtained the sample material, the lysis and extraction (if applicable) steps were completed effectively, and the included RNAse inhibitor prevented RNA degradation by RNAse in the sample. In instances where the control line is detected, the test is valid.

In certain embodiments, the results region of the lateral flow assay strip comprises a second results sub-region (e.g., a wicking area) to absorb fluid flowing through the lateral flow assay strip. Any excess fluid may flow through the second results sub-region.

As an illustrative example, a fluidic sample comprising an amplicon labeled with biotin and FITC may be introduced into a lateral flow assay strip (e.g., through a sample pad of a lateral flow assay strip). In some embodiments, as the labeled amplicon is transported through the lateral flow assay strip (e.g., through a particle conjugate pad of the lateral flow assay strip), a gold nanoparticle labeled with streptavidin may bind to the biotin label of the amplicon. In some cases, the lateral flow assay strip (e.g., a test pad of the lateral flow assay strip) may comprise a first test line comprising an anti-FITC antibody. In some embodiments, the gold nanoparticle-amplicon conjugate may be captured by the anti-FITC antibody, and an opaque band may develop as additional gold nanoparticle-amplicon conjugates are captured by the anti-FITC antibodies of the first test line. The development of said opaque band indicates the successful detection of the presence of the target nucleic acid within the sample.

In some cases, the lateral flow assay strip (e.g., a test pad of the lateral flow assay strip) further comprises a first lateral flow control line comprising biotin. In some embodiments, excess gold nanoparticles labeled with streptavidin (i.e., gold nanoparticles that were not conjugated to an amplicon) transported through the lateral flow assay strip may bind to the biotin of the first lateral flow control line, demonstrating that liquid was successfully transported to the first lateral flow control line.

In one embodiment, following amplification, processed nucleic acids (control, and if present, test) are released onto the sample pad of a lateral flow assay strip. By passive capillary flow, the nucleic acids of the sample are wicked over a conjugate pad where a visible dye attaches to the nucleic acids. As the labeled amplicons migrate across the assay strip, they pass over multiple discrete lines of immobilized antibodies. The antibodies in a given line will capture a subset of the nucleic acids (e.g., control or test) with high specificity. In this fashion, control nucleic acids are captured on a control line, test products are captured on one or more test lines. When the nucleic acids are captured on their respective lines, the dye attached to each nucleic acid generates a colored line on the assay strip. The presence of a visible Positive Control line indicates that the lateral flow test ran successfully, while the presence of the test line indicates the target nucleic acid was detected in the sample.

Multiplexed Testing

When a subject is ill with vague symptoms and/or symptoms common to a plurality of different possible ailments, determining which of several possible ailments is afflicting the subject may be time consuming, inconvenient, and expensive, especially when doctors and/or laboratory analysis is required. Moreover, if the subject is uncooperative (e.g., a young child), it may be difficult to obtain a suitable sample for a test, and this difficulty may be compounded when multiple samples are needed for multiple tests. Thus, a mechanism for testing for multiple different pathogens in a single test procedure with a single sample from the subject would be advantageous.

In some embodiments, a mechanism is provided in which a single test sample obtained from a subject may be used to test for multiple different target nucleic acids (“multiplexed testing”), and in which a user may obtain test results for the multiple different target nucleic acid sequences on a single test substrate. In some embodiments, the single test substrate may be a lateral flow assay strip on which reagents are present for indicating the presence of each of the multiple different target nucleic acid sequences. In some embodiments, the lateral flow assay strip may be pre-loaded in a test device, thus avoiding handling and possible contamination by a user. A lateral flow test with multiple detection reagents, including that of a control, is performed in one embodiment.

In some embodiments of the present technology, the lateral flow assay strip may be configured to detect two or more target nucleic acid sequences. In such embodiments, where the lateral flow test is configured for multiplexed detection (e.g., detection of multiple target nucleic acid sequences), the test region may comprise multiple test lines. In some embodiments, the test region comprises 2, 3, 4, 5, 6, 7, 8, 9, or 10 test lines and thereby may screen for the presence of 1, 2, 3, 4, 5, 6, 7, 8, 9, or 10 target nucleic acid sequences. Each test line may detect different target nucleic acids, the same target nucleic acid, or a combination thereof. For example, in some embodiments a results region may comprise two test lines, wherein the first test line is specific for a first target nucleic acid (e.g., a coronavirus nucleic acid sequence; e.g., COVID-19) and the second test line is specific for a second target nucleic acid sequence (e.g., an influenza nucleic acid sequence; e.g., influenza type A or influenza type B). In other embodiments, where a results region comprises three test lines, two of the test lines may each be specific for a same first target nucleic acid, and the third test line may be specific for a second target nucleic acid. Alternatively, each of the three test lines may be specific for a different target nucleic acid (e.g., three target nucleic acids may be detected) or each of the three test lines may be specific for the same target nucleic acid (e.g., one target nucleic acid may be detected).

Accordingly, in some embodiments a lateral flow assay strip comprises multiple test lines on a single test strip for detection of one or more pathogens. In one embodiment, the lateral flow test comprises a test line for SARS-CoV-2 and a test line for an influenza (e.g., Type A or Type B). In another embodiment, the lateral flow test comprises a test line for each of SARS-CoV-2, influenza Type A, and influenza Type B. In one embodiment, the test comprises a test line for SARS-CoV-2 and a test line for SARS-CoV-2 having a D614G mutation in its spike protein (see, e.g., Korber et al., 2020). In further embodiments, the test may be used to differentiate between infections caused by different types of pathogens, such as, for example, viral and bacterial infections.

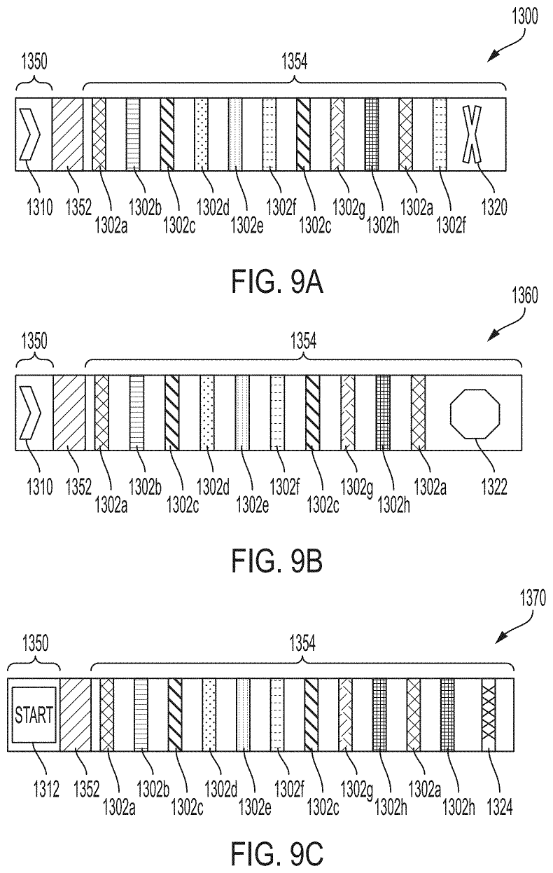

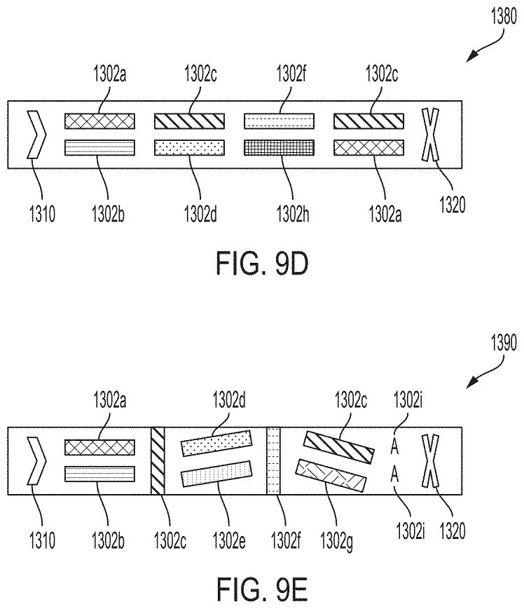

FIGS. 9A to 9E schematically show examples of lateral flow assay strips 1300, 1360, 1370, 1380, 1390 useable for multiplexed testing, according to some embodiments of the present technology. In certain embodiments, the lateral flow assay strip may comprise one or more subregions as described herein. In some instances, the lateral flow assay strip may comprise a first sub-region 1350 (e.g., a sample intake pad or region) where a fluidic sample is introduced to the lateral flow assay strip. In some instances, the lateral flow assay strip may comprise a second sub-region 1352 (e.g., a particle conjugate pad or region) comprising a plurality of labeled particles. In some cases, the particles may comprise gold nanoparticles (e.g., colloidal gold nanoparticles). The particles may be labeled with any suitable label. Non-limiting examples of suitable labels may include biotin, streptavidin, fluorescein isothiocyanate (FITC), fluorescein amidite (FAM), fluorescein, and digoxigenin (DIG).

In certain embodiments of the present technology, the lateral flow assay strip may comprise a third sub-region 1354 (e.g., a test region) comprising a plurality of test lines 1302a, 1302b . . . 1302i, each of which may detect a different target nucleic acid sequence. In some embodiments, a first test line (e.g., 1302a) may comprise a first capture reagent (e.g., an immobilized antibody) configured to detect a first target nucleic acid sequence, and a second test line (e.g., 1302b) may comprise a second capture reagent configured to detect a second target nucleic acid. In some instances, the lateral flow assay strip may include one or more duplicate test lines. For example, in FIG. 9A there are two instances of the test line 1302a so that the lateral flow assay strip may enable self-confirmation of the presence (or absence) of the first target nucleic acid sequence. In some instances, more than two test lines of the lateral flow assay strip may be configured to detect the same target nucleic acid sequence.

In certain embodiments of the present technology, the third sub-region 1354 (e.g., the test region) of the lateral flow assay strip may comprise one or more control lines 1310, 1312, 1320, 1322, 1324. The test lines 1302a, 1302b . . . 1302i and the one or more control lines 1310, 1312, 1320, 1322, 1324 may have any suitable shape or pattern (e.g., one or more straight lines, curved lines, dots, squares, check marks, x marks, geometrical shapes, alphanumeric characters, etc.), and thus the word “line” in the terms “test lines” and “control lines” may encompass a region and need not be limited to a line shape (see, e.g., 1310, 1320, 1322, 1302h). The test lines 1302a, 1302b . . . 1302i and the one or more control lines 1310, 1312, 1320, 1322, 1324 may have any orientation relative to the lateral flow assay strip, as depicted in FIGS. 9E and 9F. In certain instances, a first control line 1310, 1312 may be a human (or animal) nucleic acid control line. In some embodiments, for example, the human (or animal) nucleic acid control line 1310, 1312 may be configured to detect a nucleic acid sequence (e.g., RNase P) that may be generally present in all humans (or animals). In some cases, the human (or animal) nucleic acid control line 1310, 1312 becoming detectable may indicate that a human (or animal) sample was successfully collected, nucleic acids from the sample were amplified, and the amplicons (i.e., the amplified nucleic acids) were transported to the lateral flow assay strip.

In some embodiments of the present technology, the lateral flow assay strip may comprise a second control line 1320, 1322, 1324, which may be a liquid movement control line. The second control line 1320, 1322, 1320 becoming detectable may indicate that a liquid (e.g., the fluidic sample) was successfully transported through the lateral flow assay strip. In some embodiments, the second control line 1320, 1322, 1324 becoming detectable may indicate a completion of a reaction phase of the testing procedure.

In some embodiments of the present technology, the lateral flow assay strip may comprise two or more control lines 1310, 1312, 1320, 1322, 1324. The two or more control lines 1310, 1312, 1320, 1322, 1324 may each have any suitable shape or pattern (e.g., one or more straight lines, curved lines, dots, squares, check marks, x marks, geometric shapes, alphanumeric characters, etc.), which may be different from each other. In some instances, for example, the lateral flow assay strip may comprise a human (or animal) nucleic acid control line (e.g., 1310, 1312) and a liquid movement control line (e.g., 1320, 1322, 1324).

In some embodiments of the present technology, a front-side control line (e.g., 1310, 1312) may be present in the first sub-region 1350, and an end-side control line (e.g., 1320, 1322, 1324) may be present in the third sub-region 1354 as a final line or one of the final lines with which the fluidic sample may interact, as depicted in FIGS. 9A to 9C. For example, the plurality of test lines 1302a, 1302b . . . 1302i may be located between the front-side and end-side control lines.

As noted above, the plurality of test lines 1302a, 1302b . . . 1302i, may be configured to perform multiplexed testing to detect two or more different pathogens or target nucleic acid sequences. The front-side control line (e.g., 1310, 1312) may have a pattern or shape to indicate an inlet side of the lateral flow assay strip. The front-side control line (may be contacted by amplicon-containing fluid (e.g., the fluidic sample) before the amplicon-containing fluid contacts the plurality of test lines 1302a, 1302b . . . 1302i and the end-side control line. The end-side control line may be down-flow from the plurality of test lines 1302a, 1302b . . . 1302i, such that the end-side control line may be contacted by the amplicon-containing fluid after the plurality of test lines 1302a, 1302b . . . 1302i have been contacted. The end-side control line may serve as a control line for confirming that the amplicon-containing fluid passed over all the test lines (e.g., 1302a, 1302b . . . 1302i) of the lateral flow assay strip. The end-side control line may have a pattern or shape different from that of the front-side control line and may be located at a known distance from the front-side control line, which may facilitate reading of the lateral flow assay strip by machine vision.

### Colorimetric Test

In some embodiments, one or more target nucleic acid sequences are detected using a colorimetric test. As used herein, colorimetric “test” and colorimetric “assay” are used interchangeably, and refer to a method of detecting the presence of a target substance in a sample with the aid of a color reagent.

In some embodiments, a colorimetric assay comprises both test reagents (e.g., that turn a certain color when bound to a target nucleic acid) and stop (e.g., control) reagents (e.g., that turn a certain color when bound to a non-target nucleic acid so as to indicate a successful test). Thus, in certain embodiments a fluidic sample is exposed to a reagent that undergoes a color change when bound to a target nucleic acid (e.g., viral DNA or RNA). In some embodiments, the colorimetric assay further comprises a stop reagent, such as sulfonic acid. That is, when the fluidic sample is mixed with the reagents, the solution turns a specific color (e.g., red) if the target nucleic acid is present, thereby indicating that the sample is positive. If the solution turns a different color (e.g., green), the target nucleic acid is not present, thereby indicating that the sample is negative. In some embodiments, the colorimetric assay may be a colorimetric LAMP assay; that is, the LAMP reagents may react in the presence or absence of a target nucleic acid sequence (e.g., from SARS-CoV-2) to turn one of two colors.

In some embodiments, a colorimetric assay of the present invention is multiplexed such that multiple target nucleic acids may be detected at the same time. For example, in certain embodiments, the colorimetric assay comprises a cartridge comprising a central sample chamber in fluidic communication with a plurality of peripheral chambers (e.g., at least four peripheral chambers). In some embodiments, each peripheral chamber comprises isothermal nucleic acid amplification reagents comprising a unique set of primers (e.g., primers specific for one or more target nucleic acid sequences, primers specific for a positive test control, primers specific for a negative test control).

In some embodiments, two nucleic acids (e.g., one target and one control) are detected at the same time (if present in the sample). In some embodiments, three nucleic acids (e.g., two targets and one control) are detected at the same time (if present in the sample). In some embodiments, four nucleic acids (e.g., three targets and one control) are detected at the same time (if present in the sample). In some embodiments, five nucleic acids (e.g., four targets and one control) are detected at the same time (if present in the sample). Thus, multiple nucleic acids, including control nucleic acids, may each be detected simultaneously (if present in the same).

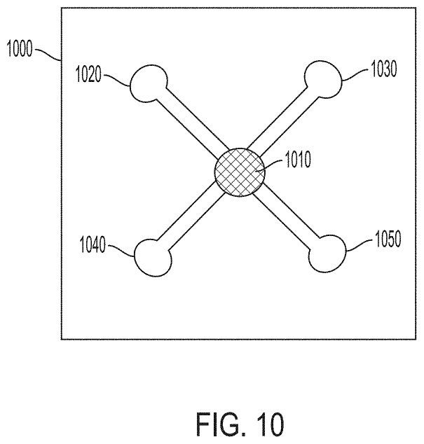

FIG. 10 shows a top-down view of exemplary colorimetric device 1000. In FIG. 10, colorimetric device 1000 comprises central sample chamber 1010, which is in fluidic communication with first peripheral chamber 1020, second peripheral chamber 1030, third peripheral chamber 1040, and fourth peripheral chamber 1050. Each of peripheral chambers 1020, 1030, 1040, and 1050 may comprise a unique set of primers. In an exemplary, non-limiting embodiment, one peripheral chamber (e.g., 1020) comprises primers specific for one or more target nucleic acid sequences (e.g., a target nucleic acid sequence of SARS-CoV-2, a SARS-CoV-2 variation, or an influenza virus). In some cases, one peripheral chamber (e.g., 1030) comprises primers specific for a positive test control (e.g., primers for RNase P). In some cases, one peripheral chamber (e.g., 1040) comprises primers specific for a second target nucleic acid sequence.

In operation, a sample may be deposited in central sample chamber 1010. In some cases, the sample may be combined with a reaction buffer in central sample chamber 1010. In some instances, central sample chamber 1010 may be heated to lyse cells within the sample. In some cases, the lysate may be directed to flow from central sample chamber 1010 to each of the plurality of peripheral chambers 1020, 1030, 1040, and 1050 comprising unique primers. In some cases, a colorimetric reaction may occur in each peripheral chamber, resulting in varying colors in the peripheral chambers. In some cases, the results within each peripheral chamber may be visible (e.g., through a clear film or other covering). Accordingly, in some embodiments successful detection of nucleic acids may in some embodiments be visualized by the user as distinct colors.

### CRISPR/Cas-Mediated Detection

In some embodiments, one or more target nucleic acid sequences are detected using CRISPR/Cas-mediated detection. CRISPR generally refers to Clustered Regularly Interspaced Short Palindromic Repeats, and Cas generally refers to a particular family of proteins. In some cases, a CRISPR/Cas-mediated detection platform can be combined with an isothermal amplification method to create a single step reaction (Joung et al., “Point-of-care testing for COVID-19 using SHERLOCK diagnostics,” 2020).

In some embodiments, CRISPR/Cas-mediated detection of one or more target nucleic acids is combined with LAMP. In some embodiments, CRISPR/Cas-mediated detection of one or more target nucleic acids is combined with RPA. In some embodiments, CRISPR/Cas-mediated detection of one or more target nucleic acids is combined with NEAR. In some embodiments, CRISPR/Cas-mediated detection comprises the addition of one or more additional reagents to the amplification procedure (e.g., LAMP, RPA, NEAR, etc.). For example, the amplification and CRISPR detection methods may be performed using reagents having compatible chemistries (e.g., reagents that do not interact detrimentally with one another and are sufficiently active to perform amplification and detection). Accordingly, in some embodiments, one or more reagents included in the step of amplification comprise one or more reagents for CRISPR/Cas detection.

CRISPR/Cas detection platforms are known in the art. Examples of such platforms include SHERLOCK® and DETECTR® (see, e.g., Kellner et al., Nature Protocols, 2019, 14: 2986-3012; Broughton et al., Nature Biotechnology, 2020; Joung et al., 2020). In some embodiments, CRISPR/Cas methods are used to detect a target nucleic acid sequence (e.g., from a pathogen). In particular, a guide nucleic acid designed to recognize a target nucleic acid sequence (e.g., a SARS-CoV-2-specific sequence) may be used to detect target nucleic acid sequences present in a sample. If the sample comprises the target nucleic acid sequence, gRNA will bind to the target nucleic acid sequence and activate a programmable nuclease (e.g., a Cas protein), which may then cleave a reporter molecule and release a detectable moiety (e.g., a reporter molecule tagged with specific antibodies, a fluorophore, a dye, a polypeptide). In some embodiments, the detectable moiety binds to a capture reagent (e.g., an antibody) on a lateral flow strip, as described herein.

In some embodiments, the one or more reagents for CRISPR/Cas-mediated detection comprise one or more guide nucleic acids. As noted above, a guide nucleic acid may comprise a segment with reverse complementarity to a segment of the target nucleic acid sequence. In some embodiments, the guide nucleic acid is selected from a group of guide nucleic acids that have been screened against the nucleic acid of a strain of an infection or genomic locus of interest. In certain instances, for example, the guide nucleic acid may be selected from a group of guide nucleic acids that have been screened against the nucleic acid of a strain of SARS-CoV-2. In some embodiments, guide nucleic acids that are screened against the nucleic acid of a target sequence of interest can be pooled. Without wishing to be bound by a particular theory, it is thought that pooled guide nucleic acids directed against a single target nucleic acid can ensure broad coverage of the target nucleic acid within a single reaction. The pooled guide nucleic acids, in some embodiments, are directed to different regions of the target nucleic acid and may be sequential or non-sequential.

In some embodiments, a guide nucleic acid comprises a crRNA and/or tracrRNA. The guide nucleic acid may not be naturally occurring and may be made by artificial combination of otherwise separate segments of sequence. For example, in some embodiments, an artificial guide nucleic acid may be synthesized by chemical synthesis, genetic engineering techniques, and/or artificial manipulation of isolated segments of nucleic acids. In some embodiments, the targeting region of a guide nucleic acid is at least 10, 15, 20, 25, 30, 35, 40, 45, 50, 55, or 60 nucleotides (nt) in length. In some embodiments, the targeting region of a guide nucleic acid has a length in a range from 10 to 20 nt, 10 to 30 nt, 10 to 40 nt, 10 to 50 nt, 10 to 60 nt, 20 to 30 nt, 20 to 40 nt, 20 to 50 nt, 20 to 60 nt, 30 to 40 nt, 30 to 50 nt, 30 to 60 nt, 40 to 50 nt, 40 to 60 nt, or 50 to 60 nt.

In some embodiments, the one or more reagents for CRISPR/Cas-mediated detection comprise one or more programmable nucleases. In some embodiments, a programmable nuclease is capable of sequence-independent cleavage after the gRNA binds to its specific target sequence. In some instances, the programmable nuclease is a Cas protein. Non-limiting examples of suitable Cas proteins include Cas9, Cas12a, Cas12b, Cas13, and Cas14. In general, Cas9 and Cas12 nucleases are DNA-specific, Cas13 is RNA-specific, and Cas14 targets single-stranded DNA.

In some embodiments, the one or more reagents for CRISPR/Cas-mediated detection comprise a plurality of guide nucleic acids and a plurality of programmable nucleases. In some embodiments, each guide nucleic acid of the plurality of guide nucleic acids targets a different nucleic acid and is associated with a different programmable nuclease. As an illustrative example, if a diagnostic test or device is configured to detect two different target nucleic acids, the one or more CRISPR/Cas reagents may comprise at least two different guide nucleic acids and at least two different programmable nucleases. If two target nucleic acids are present in a sample, then two different programmable nucleases will be activated, which will result in the release of two unique detectable moieties. Thus, in this manner, the CRISPR/Cas-mediated detection system may be used to detect more than one target nucleic acid. In some embodiments, the CRISPR/Cas-mediated detection system may be used to detect at least 2, at least 3, at least 4, at least 5, at least 6, at least 7, at least 8, at least 9, or at least 10 target nucleic acids.

### Diagnostic System Components

### Readout Device

In some embodiments, a diagnostic system comprises a readout device comprising a detection component (e.g., a lateral flow assay strip, a colorimetric assay). In certain embodiments, the detection component is a lateral flow assay strip. As described herein, the lateral flow assay strip may comprise one or more test lines configured to detect one or more target nucleic acid sequences and/or one or more control lines.

In some embodiments, the readout device is configured to receive a reaction tube (e.g., a reaction tube comprising fluidic contents, such as a sample from a subject, one or more reagents, and/or one or more buffers). In certain cases, for example, the readout device comprises a chimney comprising at least one opening configured to receive a reaction tube. In some embodiments, the chimney is in fluidic communication with the detection component. According to certain embodiments, the readout device further comprises a puncturing component configured to pierce at least a portion of the reaction tube upon insertion of the reaction tube into the readout device (e.g., such that at least a portion of any fluidic contents of the reaction tube are released and directed to flow towards the detection component). The puncturing component may comprise one or more blades, needles, or other elements capable of puncturing a reaction tube.

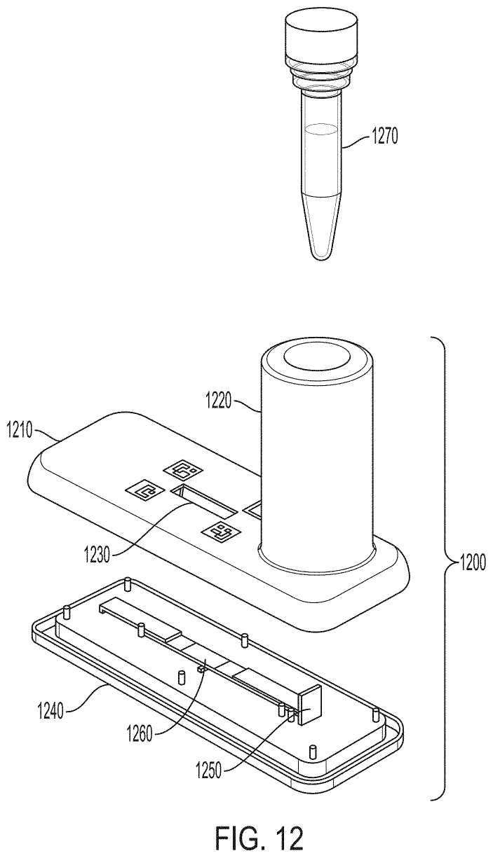

A non-limiting, illustrative embodiment of an exemplary readout device is shown in FIG. 12. In FIG. 12, readout device 1200 comprises upper component 1210, which comprises chimney 1220 and opening 1230, and lower component 1240, which comprises puncturing component 1250 and lateral flow assay strip 1260. As shown in FIG. 12, chimney 1220 may comprise an opening configured to receive a reaction tube (e.g., reaction tube 1270), and puncturing component 1250 may be located at the base of chimney 1220. Chimney 1220 may be in fluidic communication with lateral flow assay strip 1260.

Upper component 1210 and lower component 1240 may be integrally formed or may be separately formed components that are attached to each other (e.g., via one or more adhesives, one or more screws or other fasteners, and/or one or more interlocking components). When upper component 1210 and lower components 1240 are integrally formed or attached to each other, at least a portion of lateral flow assay strip 1260 may be visible through opening 1230 in upper component 1210. In some embodiments, upper component 1210 comprises one or more markings (e.g., ArUco markers) to facilitate alignment of an electronic device (e.g., a smartphone, a tablet) with opening 1230.

In operation, reaction tube 1270 comprising fluidic contents may be inserted into chimney 1220. In some embodiments, reaction tube 1270 comprises a cap (e.g., a screw-top cap, a hinged cap) and a bottom end (e.g., a tapered or rounded bottom end). In certain cases, as shown in FIG. 12, the bottom end of reaction tube 1270 is inserted into chimney 1220 prior to the cap of the reaction tube. In certain cases, the reaction tube is inverted, and the cap of reaction tube 1270 is inserted into chimney 1220 prior to the bottom end of the reaction tube. In some embodiments, upon insertion into chimney 1220, reaction tube 1270 may lock or snap into place (or may otherwise have a secure fit) such that reaction tube 1270 may not be easily removed from chimney 1270 by a user. In certain cases, locking or snapping the reaction tube into place (or otherwise preventing easy removal of reaction tube 1270 from chimney 1220) may reduce or prevent contamination.

In some embodiments, reaction tube 1270 may be punctured by puncturing component 1250 (e.g., upon insertion into chimney 1220). As a result, at least a portion of the fluidic contents of reaction tube 1270 may be directed to flow (e.g., via gravity) towards lateral flow assay strip 1260 and may come into contact with at least a portion of lateral flow assay strip 160. In some cases, at least a portion of the fluidic contents of reaction tube 1270 may be transported through lateral flow assay strip 1260 (e.g., via capillary action). In some cases, the formation (or lack of formation) of one or more visual indicators (e.g., one or more opaque lines) may indicate the presence or absence of one or more target nucleic acid sequences. In certain cases, the one or more visual indicators on lateral flow assay strip 1260 may be visible to a user through opening 1230 of upper component 1210.

The chimney, the upper component, and the lower component of the readout device may be formed from any suitable materials. In some cases, for example, the chimney, the upper component, and/or the lower component comprise one or more thermoplastic materials and/or metals. In some embodiments, the chimney, the upper component, and/or the lower component may be manufactured by injection molding, an additive manufacturing technique (e.g., 3D printing), and/or a subtractive manufacturing technique (e.g., laser cutting). In some embodiments, the upper and lower components may be sealed together. In some embodiments, the upper and lower compartments are attached to each other via one or more adhesives, one or more screws or other fasteners, and/or one or more interlocking components.

The chimney may have any suitable size and shape for receiving a reaction tube. In some embodiments, the chimney is hollow (e.g., a hollow cylinder). In certain embodiments, the chimney has an opening having an inner diameter of at least 5 mm, at least 10 mm, at least 15 mm, at least 20 mm, at least 25 mm, or at least 30 mm. In certain embodiments, the chimney has an opening having an inner diameter of 5 mm to 10 mm, 5 mm to 15 mm, 5 mm to 20 mm, 5 mm to 25 mm, 5 mm to 30 mm, 10 mm to 15 mm, 10 mm to 20 mm, 10 mm to 25 mm, 10 mm to 30 mm, 15 mm to 20 mm, 15 mm to 25 mm, 15 mm to 30 mm, or 20 mm to 30 mm. In some embodiments, the chimney has a height of 60 mm or less, 55 mm or less, 50 mm or less, 45 mm or less, 40 mm or less, 35 mm or less, 30 mm or less, 25 mm or less, 20 mm or less, 15 mm or less, or 10 mm or less. In some embodiments, the chimney has a height in a range from 10 mm to 20 mm, 10 mm to 30 mm, 10 mm to 40 mm, 10 mm to 50 mm, 10 mm to 60 mm, 20 mm to 30 mm, 20 mm to 40 mm, 20 mm to 50 mm, 20 mm to 60 mm, 30 mm to 40 mm, 30 mm to 50 mm, 30 mm to 60 mm, 40 mm to 50 mm, 40 mm to 60 mm, or 50 mm to 60 mm.

### Sample-Collecting Components

In some embodiments, the diagnostic system comprises one or more sample-collecting components. The one or more sample-collecting components may be configured to collect a sample (e.g., a nasal secretion, an oral secretion, a cell scraping, blood, urine) from a subject (e.g., a human subject, an animal subject).

In some embodiments, the sample-collecting component comprises a swab element. In certain cases, the swab element comprises an absorbent material. Non-limiting examples of suitable absorbent materials include cotton, filter paper, cellulose, cellulose-derived materials, polyurethane, polyester, rayon, nylon, microfiber, viscose, and alginate. In some instances, the swab element is a foam swab and/or a flocked swab (e.g., comprising flocked fibers of a material). In some embodiments, the swab element comprises a thermoplastic polymer (e.g., a polystyrene, a polyolefin such as polyethylene or polypropylene) and/or a metal (e.g., aluminum). In some such embodiments, the swab element may be formed by injection molding, an additive manufacturing process (e.g., 3D printing), and/or a subtractive manufacturing process (e.g., laser cutting).

In certain embodiments, at least a portion of the swab element is wrapped in a material (e.g., plastic) to ensure sterility until use. In some embodiments, the swab element is pre-moistened. The swab element may have any suitable size and shape. In some embodiments, the swab element has a relatively small diameter (i.e., largest cross-sectional dimension). In certain cases, a relatively small diameter may facilitate insertion of the swab element into a nasal cavity (e.g., anterior nares) or an oral cavity of a subject. In certain cases, a relatively small diameter may facilitate insertion of the swab element (after sample collection) into a diagnostic system component (e.g., a reaction tube, a reservoir of a cartridge, a sample port of a blister pack). In certain embodiments, the swab element has a maximum diameter of 20 mm or less, 15 mm or less, 10 mm or less, 9 mm or less, 8 mm or less, 7 mm or less, 6 mm or less, 5 mm or less, 4 mm or less, 3 mm or less, 2 mm or less, or 1 mm or less. In some embodiments, the swab element has a maximum diameter in a range from 1 mm to 2 mm, 1 mm to 5 mm, 1 mm to 10 mm, 2 mm to 5 mm, 2 mm to 10 mm, 2 mm to 15 mm, 2 mm to 20 mm, 5 mm to 10 mm, 5 mm to 15 mm, 5 mm to 20 mm, 10 mm to 15 mm, or 10 mm to 20 mm.

In some embodiments, the swab element of the sample-collecting component is proximal to a stem element (e.g., a handle, an applicator). In certain cases, the stem element facilitates collection of a sample with the swab element. In some instances, for example, the stem element facilitates insertion of the swab element into a nasal cavity (e.g., anterior nares) or an oral cavity of a subject. The stem element may be formed from any suitable material. In some embodiments, the stem element comprises a thermoplastic polymer (e.g., a polystyrene, a polyolefin such as polyethylene or polypropylene), a metal (e.g., aluminum), wood, paper, and/or another type of material. In some embodiments, the stem element comprises one or more markings and/or flanges. The markings and/or flanges may, in some instances, facilitate sample collection by indicating the appropriate depth of insertion (e.g., into a nasal cavity).

### Reaction Tube(s)

In some embodiments, at least one reagent is not contained within a diagnostic device, and a diagnostic system comprises one or more reaction tubes. The one or more reaction tubes may contain any reagent(s) described above. In some embodiments, the one or more reaction tubes comprise at least one reagent in liquid form. In some embodiments, the one or more reaction tubes comprise at least one reagent in solid form.

A reaction tube of a diagnostic system may be formed from any suitable material. In some embodiments, the reaction tube is formed from a polymer. Non-limiting examples of suitable polymers include polypropylene (PP), polytetrafluoroethylene (PTFE), polyurethane (PU), polyvinyl chloride (PVC), polystyrene, neoprene, nitrile, nylon and polyamide. In some embodiments, the reaction tube comprises glass and/or a ceramic. The glass may, in some instances, be an expansion-resistant glass (e.g., borosilicate glass or fused quartz). In some embodiments, the reaction tube is an Eppendorf tube. In some embodiments, the reaction tube has a substantially flat bottom (e.g., the reaction tube can stand on its own), a substantially round bottom, or a substantially conical bottom. If the reaction tube has a round or conical bottom, or any other bottom that does not allow the reaction tube to readily stand on its own, the diagnostic system may further comprise a stand for the reaction tube. In some embodiments, the reaction tube is sterile.

The reaction tubes, in some embodiments, further comprise at least one cap. In some embodiments, the reaction tube comprises a partially removable cap (e.g., a hinged cap) or one or more wholly removable caps (e.g., one or more screw-top caps, one or more stoppers). In some embodiments, the one or more caps comprise reagents in solid form (e.g., lyophilized, dried, crystallized, air jetted reagents).

The reaction tube may be configured to hold any suitable volume of liquid. In some embodiments, the reaction tube is configured to hold a volume of at least 5 μL, at least 10 μL, at least 15 μL, at least 20 μL, at least 25 μL, at least 30 μL, at least 40 μL, at least 50 μL, at least 60 μL, at least 70 μL, at least 80 μL, at least 90 μL, at least 100 μL, at least 150 μL, at least 200 μL, at least 250 μL, at least 300 μL, at least 400 μL, at least 500 μL, at least 600 μL, at least 700 μL, at least 800 μL, at least 900 μL, at least 1 mL, at least 1.5 mL, or at least 2 mL. In some embodiments, the reaction tube is configured to hold a volume in a range from 5 μL to 10 μL, 5 μL to 20 μL, 5 μL to 50 μL, 5 μL to 70 μL, 5 μL to 100 μL, 5 μL to 200 μL, 5 μL to 500 μL, 5 μL to 1 mL, 5 μL to 1.5 mL, 5 μL to 2 mL, 10 μL to 20 μL, 10 μL to 50 μL, 10 μL to 70 μL, 10 μL to 100 μL, 10 μL to 200 μL, 10 μL to 500 μL, 10 μL to 1 mL, 10 μL to 1.5 mL, 10 μL to 2 mL, 20 μL to 50 μL, 20 μL to 70 μL, 20 μL to 100 μL, 20 μL to 200 μL, 20 μL to 500 μL, 20 μL to 1 mL, 20 μL to 1.5 mL, 20 μL to 2 mL, 50 μL to 70 μL, 50 μL to 100 μL, 50 μL to 200 μL, 50 μL to 500 μL, 50 μL to 1 mL, 50 μL to 1.5 mL, 50 μL to 2 mL, 70 μL to 100 μL, 70 μL to 200 μL, 70 μL to 500 μL, 70 μL to 1 mL, 70 μL to 1.5 mL, 70 μL to 2 mL, 100 μL to 200 μL, 100 μL to 500 μL, 100 μL to 1 mL, 100 μL to 1.5 mL, 100 μL to 2 mL, 200 μL to 500 μL, 200 μL to 1 mL, 200 μL to 1.5 mL, 200 μL to 2 mL, 500 μL to 1 mL, 500 μL to 1.5 mL, 500 μL to 2 mL, 1 mL to 1.5 mL, or 1 mL to 2 mL.

In some embodiments, the reaction tube contains a volume of liquid (i.e., fluidic contents). In certain embodiments, the fluidic contents of the reaction tube have a volume sufficient to facilitate fluid flow through a lateral flow assay strip. In some embodiments, the fluidic contents of the reaction tube have an initial volume of at least 5 μL, at least 10 μL, at least 15 μL, at least 20 μL, at least 25 μL, at least 30 μL, at least 40 μL, at least 50 μL, at least 60 μL, at least 70 μL, at least 80 μL, at least 90 μL, at least 100 μL, at least 150 μL, at least 200 μL, at least 250 μL, at least 300 μL, at least 400 μL, at least 500 μL, at least 600 μL, at least 700 μL, at least 800 μL, at least 900 μL, at least 1 mL, at least 1.5 mL, or at least 2 mL. In some embodiments, the fluidic contents of the reaction tube have an initial volume in a range from 5 μL to 10 μL, 5 μL to 20 μL, 5 μL to 50 μL, 5 μL to 70 μL, 5 μL to 100 μL, 5 μL to 200 μL, 5 μL to 500 μL, 5 μL to 1 mL, 5 μL to 1.5 mL, 5 μL to 2 mL, 10 μL to 20 μL, 10 μL to 50 μL, 10 μL to 70 μL, 10 μL to 100 μL, 10 μL to 200 μL, 10 μL to 500 μL, 10 μL to 1 mL, 10 μL to 1.5 mL, 10 μL to 2 mL, 20 μL to 50 μL, 20 μL to 70 μL, 20 μL to 100 μL, 20 μL to 200 μL, 20 μL to 500 μL, 20 μL to 1 mL, 20 μL to 1.5 mL, 20 μL to 2 mL, 50 μL to 70 μL, 50 μL to 100 μL, 50 μL to 200 μL, 50 μL to 500 μL, 50 μL to 1 mL, 50 μL to 1.5 mL, 50 μL to 2 mL, 70 μL to 100 μL, 70 μL to 200 μL, 70 μL to 500 μL, 70 μL to 1 mL, 70 μL to 1.5 mL, 70 μL to 2 mL, 100 μL to 200 μL, 100 μL to 500 μL, 100 μL to 1 mL, 100 μL to 1.5 mL, 100 μL to 2 mL, 200 μL to 500 μL, 200 μL to 1 mL, 200 μL to 1.5 mL, 200 μL to 2 mL, 500 μL to 1 mL, 500 μL to 1.5 mL, 500 μL to 2 mL, 1 mL to 1.5 mL, or 1 mL to 2 mL.

In some embodiments, the fluidic contents of the reaction tube comprise a reaction buffer. In certain instances, the reaction buffer comprises one or more buffers. Non-limiting examples of suitable buffers include phosphate-buffered saline (“PBS”), Tris, and/or Tris-HCl. In some embodiments, the reaction buffer comprises one or more salts. Non-limiting examples of suitable salts include magnesium sulfate, ammonium sulfate, potassium chloride, potassium acetate, and magnesium acetate tetrahydrate.

In some embodiments, the fluidic contents of the reaction tube comprise one or more lysis reagents. In certain embodiments, the fluidic contents comprise a detergent. Non-limiting examples of suitable detergents include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40. In some embodiments, the fluidic contents comprise one or more enzymes and/or one or more pH-changing reagents.

In some embodiments, the fluidic contents of the reaction tube comprise one or more nucleic acid amplification reagents. In some embodiments, the fluidic contents of the reaction tube comprise one or more other reagents (e.g., a RNase inhibitor).

In one non-limiting embodiment, the fluidic contents of the reaction tube comprise 20 mM Tris-HCl (pH 8.8 at 25° C.), 0.1% (v/v) Tween 20, 8 mM magnesium sulfate, 10 mM ammonium sulfate, and 50 mM potassium chloride. In another non-limiting embodiment, the fluidic contents of the reaction tube comprise 25 mM Tris buffer, 5% (w/v) poly(ethylene glycol) 35,000 kDa, 14 mM magnesium acetate tetrahydrate, 100 mM potassium acetate, and greater than 85% volume nuclease free water.

In some embodiments, the fluidic contents of the reaction tube have a relatively neutral pH. In some embodiments, the fluidic contents of the reaction tube have a pH in a range from 5.0 to 6.0, 5.0 to 7.0, 5.0 to 7.5, 5.0 to 8.0, 5.0 to 8.5, 5.0 to 9.0, 5.0 to 9.5, 5.0 to 10.0, 6.0 to 7.0, 6.0 to 7.5, 6.0 to 8.0, 6.0 to 8.5, 6.0 to 9.0, 6.0 to 9.5, 6.0 to 10.0, 7.0 to 8.0, 7.0 to 8.5, 7.0 to 9.0, 7.0 to 9.5, 7.0 to 10.0, 8.0 to 9.0, 8.0 to 9.5, 8.0 to 10.0, or 9.0 to 10.0.

The fluidic contents of the reaction tube may have any suitable volume. In some embodiments, the volume of the fluidic contents of the reaction tube is at least about 500 μL, at least about 750 μL, 1 mL, 1.5 mL, 1.65 mL, or at least about 2 mL. In some embodiments, the volume of the fluidic contents of the reaction tube is in a range from 500 μL to 750 μL, 500 μL to 1 mL, 500 μL to 1.5 mL, 500 μL to 1.65 mL, 500 μL to 2 mL, 750 μL to 1 mL, 750 μL to 1.5 mL, 750 μL to 1.65 mL, 750 μL to 2 mL, 1 mL to 1.5 mL, 1 mL to 1.65 mL, 1 mL to 2 mL, 1.5 mL to 1.65 mL, or 1.5 mL to 2 mL.

### Caps

In some embodiments, the diagnostic system comprises at least one cap comprising one or more reagents. The one or more reagents may be any reagents described herein (e.g., lysis, nucleic acid amplification, decontamination, and/or stabilization reagents). In certain embodiments, the cap is a caged cap comprising a cap portion and a cage portion attached to the cap portion. The cage portion may be structured to retain one or more reagents, and the cap portion may be structured to cover an opening of a reaction vessel (e.g., a reaction tube, a reaction chamber, etc.). As described herein, the one or more reagents may comprise a lyophilized material solidified into a desired form (e.g., a bead, a tablet, a pellet etc.) that fits in the cage, in some embodiments. An amount of the lyophilized material appropriate for a test procedure may be included in each solidified form of the lyophilized material. In some embodiments, the one or more reagents may comprise particulates (e.g., powder) or a liquid surrounded by a dissolvable covering (e.g., a shell, a capsule, a gelcap, etc.) containing the particulates or the liquid therein.

In some embodiments, the cage may have an open structure that permits fluid to flow into the cage to interact with the one or more reagents but does not permit easy removal of the one or more reagents from the cage. In some such embodiments, the one or more reagents (e.g., in the form of a lyophilized bead, tablet, pellet, etc.) may be dissolved in place without being released. In some embodiments, the cage releasably holds one or more reagents. In certain instances, for example, the cage may have a deformable structure that a user may controllably deform to release the reagent(s) into, e.g., a reaction vessel, but without the user directly contacting or handling the reagent(s).

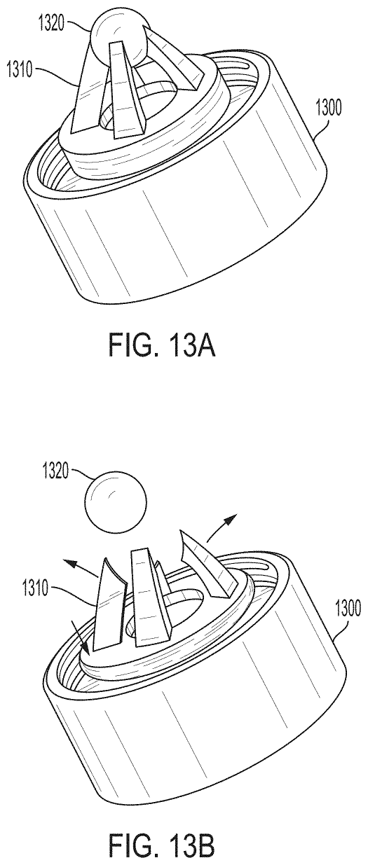

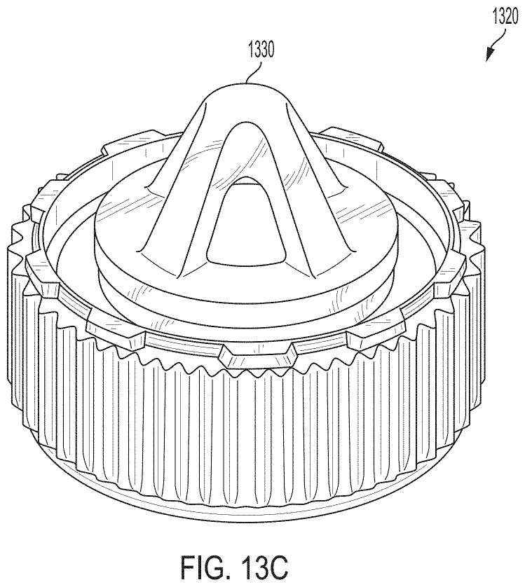

FIGS. 13A-13B are schematic illustrations of an exemplary caged cap that releasably holds one or more reagents. In FIG. 13A, caged cap 1300 comprises a plurality of deformable fingers 1310, which hold lyophilized bead 1320 within a cage. In FIG. 13B, the plurality of deformable fingers 1310 have been deformed such that lyophilized bead 1310 has been released from the cage.

FIG. 13C is a schematic illustration of an exemplary non-releasable-reactant caged cap. As shown in FIG. 13C, caged cap 1320 comprises cage portion 1330, which does not permit easy removal of any reagents located within the cage portion.

### Heater

The diagnostic system, in some embodiments, comprises a heater. In some embodiments, the diagnostic system comprises a separate heater (i.e., a heater that is not integrated with other system components). The heater may be a single-well heater or a multi-well heater. In some cases, the heater comprises a battery-powered heat source, a USB-powered heat source, a hot plate, a heating coil, and/or a hot water bath. In certain embodiments, the heating unit is contained within a thermally-insulated housing to ensure user safety. In certain instances, the heating unit is an off-the-shelf consumer-grade device. In some embodiments, the heat source is a thermocycler or other specialized laboratory equipment known in the art. In some embodiments, the heater is configured to receive a reaction tube.

In certain embodiments, the heater is integrated with the diagnostic device. In some instances, for example, the heater is a printed circuit board (PCB) heater. The PCB heater, in some embodiments, comprises a bonded PCB with a microcontroller, thermistors, and/or resistive heaters. In some embodiments, the PCB heater is in thermal communication with at least a portion of a readout device.

In some embodiments, the heater is configured to heat one or more components of a diagnostic system (e.g., fluidic contents of a reaction tube) at a temperature of at least 37° C., at least 40° C., at least 45° C., at least 50° C., at least 55° C., at least 60° C., at least 63.5° C., at least 65° C., at least 70° C., at least 75° C., at least 80° C., at least 85° C., or at least 90° C. In some embodiments, the heater is configured to heat one or more components of a diagnostic system (e.g., fluidic contents of a reaction tube) at a temperature in a range from 37° C. to 60° C., 37° C. to 70° C., 37° C. to 80° C., 37° C. to 90° C., 40° C. to 60° C., 40° C. to 70° C., 40° C. to 80° C., 40° C. to 90° C., 50° C. to 60° C., 50° C. to 70° C., 50° C. to 80° C., 50° C. to 90° C., 60° C. to 70° C., 60° C. to 80° C., 60° C. to 90° C., 70° C. to 80° C., 70° C. to 90° C., or 80° C. to 90° C.

In some embodiments, the heater is configured to heat one or more components of a diagnostic system (e.g., fluidic contents of a reaction tube) at a temperature for at least 5 minutes, at least 10 minutes, at least 15 minutes, at least 30 minutes, at least 45 minutes, at least 60 minutes, or at least 90 minutes. In certain embodiments, the heating unit is configured to heat one or more components of a diagnostic system (e.g., fluidic contents of a reaction tube) at a desired temperature for a time in a range from 5 minutes to 10 minutes, 5 minutes to 15 minutes, 5 minutes to 30 minutes, 5 minutes to 45 minutes, 5 minutes to 60 minutes, 5 minutes to 90 minutes, 10 minutes to 15 minutes, 10 minutes to 30 minutes, 10 minutes to 45 minutes, 10 minutes to 60 minutes, 10 minutes to 90 minutes, 15 minutes to 30 minutes, 15 minutes to 45 minutes, 15 minutes to 60 minutes, 15 minutes to 90 minutes, 30 minutes to 45 minutes, 30 minutes to 60 minutes, 30 minutes to 90 minutes, or 60 minutes to 90 minutes.

In some embodiments, the heater comprises at least two temperature zones. In certain instances, for example, the heater is an off-the-shelf consumer-grade heating coil connected to a microcontroller that is used to switch between two temperature zones. In some embodiments, the first temperature zone is in a range from 60° C. to 100° C., 60° C. to 90° C., 60° C. to 80° C., 60° C. to 70° C., or 60° C. to 65° C. In certain cases, the first temperature zone has a temperature of approximately 65° C. In some embodiments, the second temperature zone is in a range from 30° C. to 40° C. In certain cases, the second temperature zone has a temperature of approximately 37° C.

In some embodiments, the heater is configured to heat one or more components of a diagnostic system (e.g., fluidic contents of a reaction tube) to a plurality of temperatures for a plurality of time periods. In some embodiments, for example, a heater is configured to heat one or more components of a diagnostic system (e.g., fluidic contents of a reaction tube) at a first temperature for a first period of time and at a second temperature for a second period of time. The first temperature and the second temperature may be the same or different, and the first period of time and the second period of time may be the same or different.

In some embodiments, the heater is pre-programmed with one or more protocols. In some embodiments, for example, the heater is pre-programmed with a lysis heating protocol and/or an amplification heating protocol. A lysis heating protocol generally refers to a set of one or more temperatures and one or more time periods that facilitate lysis of the sample. An amplification heating protocol generally refers to a set of one or more temperatures and one or more time periods that facilitate nucleic acid amplification. In some embodiments, the heater comprises an auto-start mechanism that corresponds to the temperature profile needed for lysis and/or amplification. That is, a user may insert a reaction tube into the heater, and the heater may automatically run a lysis and/or amplification heating protocol. In some embodiments, the heater is controlled by a mobile application.

### Dropper

In some embodiments, the diagnostic system comprises a dropper configured to dispense an amount of a liquid (e.g., a diluent). In certain embodiments, the liquid comprises a buffer. Non-limiting examples of suitable buffers include Tris, Tris-HCl, and PBS. The dropper may be configured to dispense any amount of liquid. In some embodiments, the dropper is configured to dispense at least 10 μL, at least 20 μL, at least 50 μL, at least 100 μL, at least 200 μL, at least 250 μL, at least 300 μL, at least 350 μL, at least 400 μL, or at least 500 μL of liquid. In some embodiments, the dropper is configured to dispense an amount of liquid having a volume in a range from 10 μL to 20 μL, 10 μL to 50 μL, 10 μL to 100 μL, 10 μL to 200 μL, 10 μL to 250 μL, 10 μL to 300 μL, 10 μL to 400 μL, 10 μL to 500 μL, 50 μL to 100 μL, 50 μL to 200 μL, 50 μL to 250 μL, 50 μL to 300 μL, 50 μL to 400 μL, 50 μL to 500 μL, 100 μL to 200 μL, 100 μL to 250 μL, 100 μL to 300 μL, 100 μL to 400 μL, 100 μL to 500 μL, 200 μL to 300 μL, 200 μL to 400 μL, 200 μL to 500 μL, 300 μL to 400 μL, 300 μL to 500 μL, or 400 μL to 500 μL. In some embodiments, the diagnostic system does not comprise a dropper, and the diagnostic method does not comprise a step of dispensing an amount of liquid into a readout device (e.g., a chimney of a readout device).

### Pipette

In some embodiments, the diagnostic system comprises a pipette configured to transfer an amount of liquid. The pipette may be configured to transfer any amount of liquid. In some embodiments, the pipette is configured to transfer at least 50 μL, at least 100 μL, at least 200 μL, at least 250 μL, at least 300 μL, at least 350 μL, or at least 400 μL of liquid. In certain embodiments, the pipette is configured to transfer an amount of liquid having a volume in a range from 50 μL to 100 μL, 50 μL to 200 μL, 50 μL to 250 μL, 50 μL to 300 μL, 50 μL to 400 μL, 100 μL to 200 μL, 100 μL to 250 μL, 100 μL to 300 μL, 100 μL to 400 μL, 200 μL to 250 μL, 200 μL to 300 μL, 200 μL to 400 μL, 250 μL to 300 μL, 250 μL to 400 μL, or 300 μL to 400 μL. In some embodiments, the diagnostic system does not comprise a pipette, and the diagnostic method does not comprise a step of transferring an amount of liquid from a first reaction tube to a second reaction tube.

### Cartridge

In some embodiments, a diagnostic system comprises a cartridge (e.g., a microfluidic cartridge). In some embodiments, the cartridge comprises a body comprising one or more reagent reservoirs connected via one or more fluidic channels. A reagent reservoir generally refers to a reservoir comprising one or more reagents (e.g., lysis reagents, nucleic acid amplification reagents) and/or one or more buffers. The one or more reagents may be in solid form (e.g., lyophilized, dried, crystallized, air jetted) or liquid form. In certain embodiments, the cartridge comprises a first reagent reservoir comprising a first set of reagents (e.g., lysis reagents) and a second reagent reservoir comprising a second set of reagents (e.g., nucleic acid amplification reagents). In certain embodiments, the cartridge further comprises one or more additional reagent reservoirs comprising one or more additional sets of reagents and/or buffers (e.g., a dilution buffer). In some embodiments, the cartridge further comprises one or more gas expansion reservoirs and/or vent paths in fluidic communication with at least reagent reservoir. In some embodiments, the one or more gas expansion reservoirs and/or vent paths are configured to maintain a desired pressure in at least one reagent reservoir.

In some embodiments, the cartridge further comprises a lateral flow assay strip in fluidic communication with at least one of the one or more reagent reservoirs via one or more fluidic channels. In some instances, the lateral flow assay strip comprises a lateral flow assay strip configured to detect one or more target nucleic acids (e.g., one or more target nucleic acids from one or more pathogens).

In some embodiments, the cartridge further comprises a pumping tool configured to facilitate fluid flow to and from one or more reagent reservoirs. In some cases, the pumping tool comprises a peristaltic pump (e.g., a roller pump) and/or a reciprocating pump. In some cases, the cartridge comprises one or more pump lanes along which a user can move the pumping tool. In some embodiments, the one or more pump lanes comprise one or more valves (e.g., passive valves) and/or bypass sections. In some instances, the one or more pump lanes are configured to permit fluid flow in only one direction.

In some embodiments, the cartridge comprises an integrated heater (e.g., a PCB heater).

### Blister Pack

In some embodiments, a diagnostic system comprises one or more blister packs. In some embodiments, a blister pack comprises one or more chambers. In some cases, each chamber may comprise one or more reagents (e.g., lysis reagents, nucleic acid amplification reagents) and/or one or more buffers (e.g., dilution buffer). In certain, a chamber may be separated from an adjacent chamber by a breakable seal (e.g., a frangible seal) or a valve (e.g., a rotary valve). Diagnostic devices and systems described herein may comprise any number of blister packs, arranged in such a way so as to process a sample as described herein. In some embodiments, the blister packs comprise one or more seals (e.g., differential seals, frangible seals) that allow reagents to be delivered in a controlled manner (e.g., using differential seal technology). In some embodiments, the blister packs comprise one or more chambers, where each chamber comprises one or more reagents. In certain embodiments, one or more chambers store one or more reagents in solid form (e.g., lyophilized, dried, crystallized, air jetted), and one or more chambers store one or more reagents and/or buffers in liquid form. In some cases, a chamber comprising one or more reagents in solid form may be separated from a chamber comprising one or more reagents and/or buffers in liquid form by a seal (e.g., a frangible seal). In some cases, breaking the frangible seal may result in the solid reagents being suspended in the one or more liquid reagents and/or buffers. In some cases, the suspended solid reagents may be added to a sample. In some embodiments, the delivery of each reagent in a blister pack is fully automated. For example, the user may insert a sample in a sample collection region of the blister pack and then activate the blister pack. Upon activation, all of the reagents may be added to the sample in the correct amount and at the appropriate time, such that the sample is processed as described herein. In some embodiments, the blister pack further comprises a detection component (e.g., a lateral flow assay strip, a colorimetric assay). The detection component may alert the user as to whether the sample was positive or negative for the target nucleic acid sequence.

### Methods of Making the Diagnostic System Components

Certain aspects are directed to methods of making one or more components of a diagnostic system. In some embodiments, a method of making a diagnostic system comprises making a readout device. In some embodiments, a readout device comprises an upper component comprising a chimney and an opening (e.g., to allow visualizing of an underlying lateral flow strip) and a lower component comprising a lateral flow strip and one or more puncturing components. In some embodiments, a method of making a readout device comprises affixing a lateral flow strip and/or a puncturing component to a lower component. In some embodiments, the method of making the readout device further comprises attaching an upper component to a lower component via one or more adhesives, one or more screws or other fasteners, and/or one or more interlocking components. In some embodiments, a method of making a diagnostic system further comprises providing any diagnostic system component described herein (e.g., a sample-collecting component, a reaction tube, a dropper, a pipette). In certain embodiments, the method of making a diagnostic system further comprises filling a dropper with an amount of a buffer.

### Software

### Downloadable Software Application

In some embodiments, a rapid diagnostic test of the present invention is guided by a downloadable software application that detects the presence of the target nucleic acid(s). In some embodiments, the software application guides a user through steps to administer a rapid diagnostic test as contemplated herein. The software application may therefore guide the user through the steps of setting up the test components (e.g., test kit components), collecting a sample from the subject (e.g., a human, such as a patient being tested for a disease), processing the sample, and/or analyzing the sample with a detection component.

In some embodiments, the readout of the rapid diagnostic test may be presented by the software application to the user. In some embodiments, a test reading of the sample analysis (e.g., as displayed on a lateral flow assay strip), may be read or may be uploadable to a smart device or communicated through a network. In an embodiment, the software application provides a user with an application for entering the reading. Alternatively, an image of the reading may be uploaded. The software application, in an embodiment, analyzes the reading and provides a test result.

In an embodiment, the software application can be downloaded to a device. In some embodiments, the device executes a software application associated with the rapid diagnostic test. In an embodiment, the device is a computer, a tablet, and/or a smart device. The smart device can be a smartphone, a smartwatch, and/or a smart home device.

The software application can provide the instructions and/or step(s) using any form recognizable by one of ordinary skill in the art as a suitable vehicle for containing such instructions. In an embodiment, the software application uses audio, sensory, and/or visual techniques to guide a user through the test, including but not limited to user interfaces, images, sounds, lights, haptic feedback, and/or the like. For example, the instructions may be written or published, verbal, audible (e.g., telephonic), digital, optical, visual (e.g., videotape, DVD, etc.) or electronic communications (including Internet or web-based communications).

In some embodiments, the instructions instruct a user on beginning and/or ending heating protocols. In some cases, a user may receive an alert (e.g., on a mobile application) when a heating protocol (e.g., a lysis heating protocol, an amplification heating protocol) is complete. In some embodiments, the software-based application may be connected (e.g., via a wired or wireless connection) to one or more components of a diagnostic system. In certain embodiments, for example, a heater may be controlled by a software-based application. In some cases, a user may select an appropriate heating protocol through the software-based application. In some cases, an appropriate heating protocol may be selected remotely (e.g., not by the immediate user). In some cases, the software-based application may store information (e.g., regarding temperatures used during the processing steps) from the heater.

The software application can be used to validate one or more steps of the test process were performed correctly. In an embodiment, the downloadable software application confirms that the one or more reagents were added in a correct order. In an embodiment, the downloadable software application confirms that the one or more reagents were added at a correct time. In an embodiment, the downloadable software application uses a camera function to validate a color of a solution formed by adding the one or more reagents to the sample.

In an embodiment, the downloadable software application provides a user with the ability to enter a test reading or result. The test can be self-read, read by another, or uploaded to a device containing the software application for automatic reading. In an embodiment, a user manually enters the reading or result to the downloadable software application. For example, through an image or user interface appearing on a device containing the software application, a user can tap the number of lines (bands) appearing positive on the readout strip and the software application will automatically read the results.

Alternatively, a user may take an image of the readout strip and upload that image to the device containing the software application for automatic reading of the test results. In some embodiments, a marked outline is displayed on the portable electronic device to help the user align the image to the detection component of the diagnostic test prior to capture. In some embodiments, the user may capture the photo by selecting a button (e.g., a camera icon). In some embodiments, the image may be captured automatically (e.g., when the detection component is aligned with a corresponding outline, or when the detection component is automatically detected in the image). In an embodiment, the test reading and/or result is uploaded through a wireless connection.

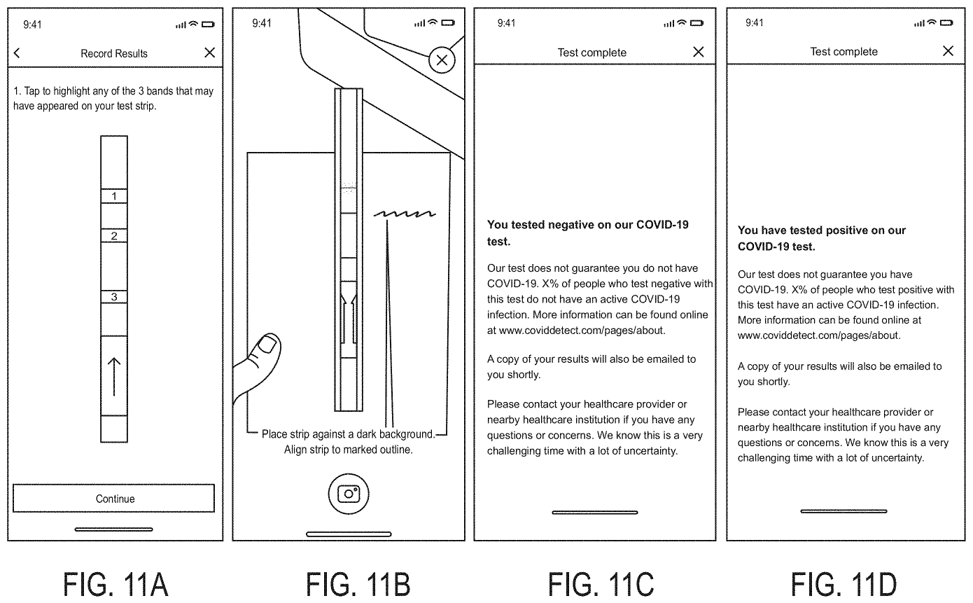

FIGS. 11A-D show images from an exemplary software application for reporting and analyzing results. FIG. 11A shows a “Record Results” screen, FIG. 11B shows an “Image Acquisition” screen, and FIGS. 11C and 11D show a “Test Complete” screen for a negative result (FIG. 11C, a sample not containing a target nucleic acid) and a positive result (FIG. 11D, a sample containing a target nucleic acid).

### Software-Based Testing Ecosystem

Additionally or alternatively to the downloadable software application described above, in some embodiments the readout of the rapid diagnostic test is integrated into a software-based testing ecosystem. Such ecosystem may therefore, in some embodiments, be configured to integrate test readings, test results, and/or other information. In an embodiment, the testing ecosystem stores test information and other information in a central database, and can disseminate information to other devices, including clinician databases/devices, agency databases/devices, medical record databases/devices, and/or the like. In an embodiment, the ecosystem can integrate aspects of patient health relating to disease progression. In an embodiment, the testing ecosystem can incorporate data from other data sources, including data provided by the users and/or data available from other data sources (e.g., clinician databases/devices, agency databases/devices, medical record databases, and/or the like). In an embodiment, the testing ecosystem stores tracking data for users of the testing ecosystem that the testing ecosystem can use to provide additional services (e.g., contact tracing).

In some embodiments, a software application can process test results (e.g., read, receive, analyze and/or generate the test results) and upload the results to the software-based ecosystem. Test results may be uploaded, manually or automatically, to the device and/or to a networked device. In an embodiment, the test reading and/or result is uploadable to a device running the downloadable software application which can upload the reading or result to the ecosystem.

In some embodiments, the ecosystem includes a device that downloads a software application that, when executed by the device, is configured to guide a user through administration of the testing. In some embodiments, the software application is further configured to upload test results and/or other subject data (e.g., subject age, subject health information, subject location data, other testing data, and/or the like), manually or automatically, to one or more of the components of the ecosystem. In some embodiments, the device can be in communication with the component(s) through the network and/or may be in direct wired and/or wireless communication with the component(s).

In some embodiments, the components of the ecosystem include one or more resources, which may include storage and/or computing resources. According to some embodiments, the resource is used to aggregate subject data, including testing data as well as other data (e.g., from the rapid diagnostic test and/or other test(s)). According to some embodiments, the resource is a remote server, a back-end server, a cloud resource, and/or the like. In some embodiments, the storage of the resource can provide a central database for the ecosystem that can be used to store user information, account information, medical information, and/or the like.

In some embodiments, the components of the ecosystem also include other computing resources, including one or more medical record databases, one or more clinician databases, one or more agency databases (e.g., the Center for Disease Control, state and/or federal authorities), and/or one or more test record databases (e.g., HIPAA-compliant databases). In an embodiment, users of the database(s) can access the databases via user devices. In some embodiments, the ecosystem can allow a device to communicate test results and/or other data directly to user devices, such as directly to a clinician device (e.g., without needing to store the data in the clinician database).

In some embodiments, the ecosystem can receive test record data. The test record data can include data for one or more other tests. The other tests can include, for example, tests performed by a third party, such as tests performed at a testing site, tests performed by a clinician, etc. In some embodiments, the test record data can include antibody test data, COVID-19 test data, influenza test data, and/or target nucleic acid test data. In some embodiments, the ecosystem can receive clinician data. The clinician data can include patient health data, historical patient data, medical examination data, surgical data, patient medical records, and/or any other clinical patient data. In some embodiments, the ecosystem can receive tracking data. In an embodiment, the tracking data can include user location data, residence data, address data, GPS-based data, and/or other tracking data available for a user.

In some embodiments, users of the ecosystem can create accounts with the ecosystem and store information within the ecosystem. In some embodiments, the software application is configured to guide a subject through setting up an account with the ecosystem (e.g., where the subject may be the user, for a self-administered test) and performing a test. In some embodiments, the user can create and/or sign into an account with the testing ecosystem. The user can manage information associated with the user's account, including personal data, healthcare data, and/or other data. The personal data can include the user's name, social security number, date of birth, address, phone number, email address, medical history, medications, and/or the like. The healthcare data can specify one or more clinicians of the user (e.g., physician(s), psychiatrist(s), psychologist(s), nurse(s), and/or other doctors or medical professionals that care for the user). The healthcare data can include data provided by the user of the software application. In some embodiments, the software application can provide forms for users to enter healthcare information, can allow a user to take an image of healthcare information, and/or the like, to provide the information to the ecosystem.

### Kits

Any of the rapid diagnostic tests described herein may formulated as a kit. As used herein a “kit” comprises a package or an assembly including one or more of the test compositions of the invention. Any one of the kits provided herein may comprise any number of reaction tubes, wells, chambers, or other vessels. Each of the components of the kit (e.g., reagents) may be provided in liquid form (e.g., in solution) or in solid form (e.g., a dried powder, lyophilized).

A kit may, in some cases, include instructions in any form that are provided in connection with the compositions of the invention in such a manner that one of ordinary skill in the art would recognize that the instructions are to be associated with the compositions of the invention. The instructions may include instructions for performing any one of the tests provided herein. The instructions may include instructions for the use, modification, mixing, diluting, preserving, administering, assembly, storage, packaging, and/or preparation of the compositions and/or other compositions associated with the kit. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications). In some embodiments, the instructions are provided as part of a software-based application, as described herein. Several exemplary kits and methods of using them are described below.

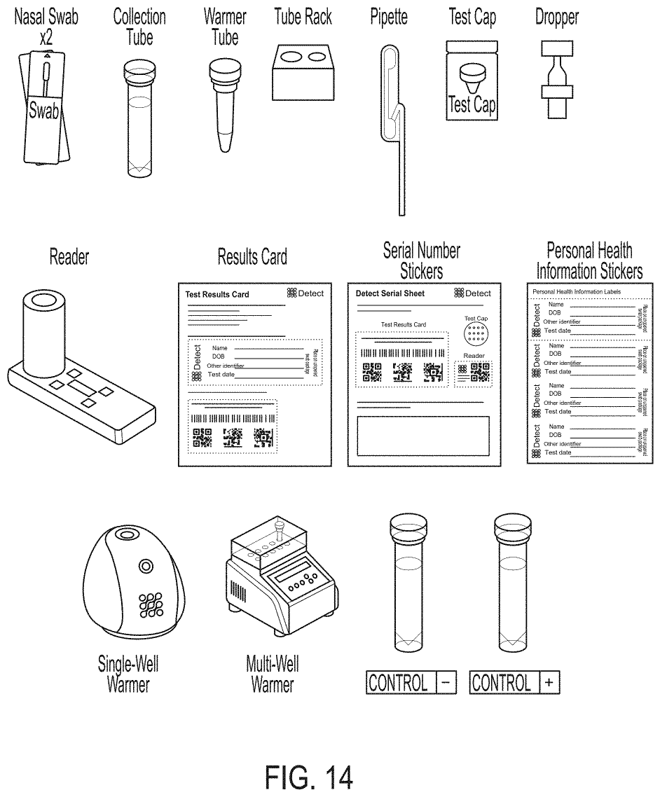

FIG. 14 depicts embodiments of certain components of a rapid diagnostic test kit, such as nasal swabs, a collection tube, a warmer tube, a tube rack, a pipette, a test cap, a dropper, a reader apparatus, a results card, serial number stickers, and personal health information stickers. In some embodiments, the kit may further comprise a single-well warmer, a multi-well warmer, a negative control, and a positive control.

Further kits are also included. In some embodiments, the kit comprises a sterile swab. After taking a nasal (anterior nares) or cheek swab sample, the swab is inserted into a sample tube containing a volume of rehydration (collection) buffer (e.g., 500 μL of PBS) and mixed around for 10 seconds. The swab is removed, and a lysis cap is added to the sample tube. The lysis cap comprises an UDG (thermolabile Uracil DNA glycosylase) lyophilized bead, which is exposed to the solution as the sample tube is inverted. After the bead has been fully dissolved (e.g., a 10-minute incubation at room temperature), the sample tube is placed in a heater at 37° C. for three minutes, and then ramped up to 65° C. and held there for 10 minutes. The temperature is then reduced back down to 37° C., which permanently denatures the UDG and simultaneously lyses cells and viral particles in the specimen, releasing their RNA. The lysis cap is removed and is replaced by an amplification cap. The amplification cap comprises a reverse transcriptase and RPA lyophilized bead. The sample tube, now comprising the amplification cap, is then inverted until the bead dissolves, reverse-transcribing the sample to cDNA and subsequently amplifying directly from the specimen in the buffer (e.g., without the need for a separate RNA extraction and purification step). Then, the sample tube is heated to 37° C. for 10-15 minutes for amplification. Then, in some embodiments, a dilution buffer is added.

In other embodiments, the sample tube is added to a readout device, and the addition of dilution buffer occurs with the readout device. In another embodiment, the amplification step utilizes LAMP reagents and dual hapten probes, and does not require dilution. The readout device then runs the same through a lateral flow test, and the results of the test (e.g., positive or negative for the target nucleic acids screened) are reported in a mobile app. In some embodiments, the sample tube is manually clicked into one end of the readout device, causing the contents of the test tube to flow through the lateral flow strip. The readout device comprises a clear window so that the user can view the lateral flow strip and the test results. In some embodiments, the readout device further comprises markings near the window so that the companion mobile app can register and acquire an image in order to process the results. The user waits five minutes, and then selects the band-pattern that is present on the lateral flow strip, using the mobile app as a guide.

In another embodiment, the kit comprises a sterile swab, a cap, an amplification cap, a heating device, and a readout device. After taking a nasal or cheek swab sample, the swab is inserted into a sample tube containing a volume of rehydration buffer (e.g., 500 μL of PBS) and mixed around for 10 seconds. The swab is removed, and a cap is added to the sample tube. The tube is then placed in the heating device (such as a USB-powered heating device) at 37° C. for three minutes, and then ramped up to 65° C. and held there for 10 minutes. The temperature is then reduced back down to 37° C. The cap is removed and is replaced by an amplification cap.

In some embodiments, the amplification cap comprises a foil seal top that is punctured or removed when the cap is placed on the tube, exposing the lyophilization bead to the solution. The amplification cap comprises a reverse transcriptase and RPA lyophilized bead. In other embodiments, the amplification cap comprises a lyophilized version of LAMP reagents. The sample tube, now comprising the amplification cap, is then inverted until the bead dissolves. Then, the sample tube is heated to 37° C. for 15 minutes for amplification, for example, using a USB-powered heating device. Then, in some embodiments, the sample tube is added to a readout device, and the readout device then runs the same through a lateral flow test, and the results of the test (e.g., positive or negative for the target nucleic acids screened) using ARUCO markers are reported in a mobile app. In some embodiments, the readout device dilutes the sample, if needed, prior to running the lateral flow test. In other embodiments, dilution is not necessary because an alternative probe, such as a dual-hapten probe described herein, has been used.

In another embodiment, the kit comprises a tube comprising UDG reagents, a cap comprising amplification reagents, a heating device, and a readout device. Optionally, the kit further comprises a swab, such as a foam swab. The user takes a sample, for example, a saliva sample or a nasal swab, and adds the sample to the tube. A cap is applied to the tube and then the tube is placed in the heating device for UDG treatment (to prevent potential cross-contamination) and lysis. In some embodiments, the heating device begins blinking when the tube is placed in the device, indicating that the heating and cooling protocol is occurring (e.g., 37° C. for three minutes, then ramped up to 65° C. for 10 minutes, and then reduced back down to 37° C.). When the heating device stops blinking, the user then removes the tube from the heating device. The user removes the cap from the tube and replaces it with the cap comprising amplification reagents (e.g., LAMP-associated reagents or RPA-associated reagents). By adding the cap, the amplification reagents are then added to the tube, where they contact the lysed sample.

In some embodiments, the amplification reagents are present in the cap as an amplification pellet, and connecting the cap to the tube releases the pellet into the tube. The user then shakes the tube briefly to mix the components, and then places it back in the heating device. The device, in some embodiments, begins flashing, as it runs the amplification protocol. As an example, if RPA reagents are used, the heating device heats the sample to 32° C. for 3 minutes, 65° C. for 10 minutes, and then cools the sample to 37° C. for 15 minutes. As another example, if LAMP reagents are used, the heating device heats the sample to 32° C. for 3 minutes, 65° C. for 40 minutes, and then cools the sample to a temperature less than 40° C. After the device stops blinking—indicating that amplification is complete—the user removes the tube from the heating device and runs the sample through a readout device (e.g., a lateral flow test designed to screen for COVID-19 and influenza). In some embodiments, the results of the test are interpreted and/or provided by a companion mobile application described herein.

As will be understood, a rapid diagnostic test kit of the present disclosure may comprise any one or more of the individual test kit components as described herein, and such components may be provided in any combination, in a single package, in multiple packages, etc.

### EXAMPLES

### Example 1—Performance of Exemplary Rapid Diagnostic Test

Aspects of the invention relate to a rapid diagnostic test capable of detecting SARS-CoV-2 nucleic acids in a patient sample. This Example investigated the performance of an exemplary qualitative Reverse Transcription Loop-mediated Isothermal Amplification (RT-LAMP) test that detected SARS-CoV-2 viral RNA from self-collected nasal swabs and indicated the result via a pattern of bands apparent on a lateral flow strip (referred to as the “Detect test”).

### Detect Test Components

The Detect test evaluated in this Example included the following components (shown in FIG. 14):
Two flocked nasal swabs. The swabs were sterilized by the manufacturer using an ethylene oxide sterilization procedure and individually packaged.
One Collection Tube containing Collection Buffer (details provided in Table 4)
One Warmer Tube (empty)
One Tube Rack
One fixed volume transfer Pipette
One Test Cap containing a lyophilized bead (details provided in Table 5)
One Dropper containing a buffer liquid designed for the Reader's lateral flow strip
One Reader containing a lateral flow strip
One Results Card for recording test results
One sheet of Serial Number Stickers for labeling the Reader, Results Card, and Test Cap
One sheet of Personal Health Information Stickers for recording patient information and labeling the Test Results Card
A reusable Single-Well Warmer or Multi-Well Warmer. The Detect Single-Well Warmer (Model 21101) was pre-programmed to automatically perform the heating protocol required to run the Detect test upon Warmer Tube insertion. After the heating protocol was complete, the Warmer beeped and its lights blinked to indicate that the reaction was complete. The Detect Multi-Well Warmer (Model 21091) fit 10 Detect Warmer Tubes and was pre-programmed to perform the heating protocol required to run the Detect test concurrently in all 10 wells after the operator pressed the start button. After the heating protocol was complete, the Warmer beeped and displayed “Finished” to indicate that the reaction was complete.

In order for the Warmer Tube to be inserted into the Reader, the Reader needed to be oriented properly (upright). After tube insertion, the performance of the Reader was independent of its orientation—the liquid flowed through the lateral flow strip driven by capillary forces which were independent of orientation.

TABLE 4
Collection Buffer Composition

| Detect Collection Buffer (1.65 mL per | |
|---|---|
| Collection Tube) | Final Composition in Reaction |
| Tris-HCl pH 8.8 at 25° C. | 20 mM |
| Tween 20 | 0.1% (v/v) |
| Magnesium Sulphate | 8 mM |
| Ammonium Sulphate | 10 mM |
| Potassium Chloride | 50 mM |

TABLE 5
Lyophilized Bead Composition

| | Final Composition |
|---|---|
| Lyophilized Bead Component | in Reaction |
| Thermolabile Uracil DNA Glycosylase | 0.02 U/μL |
| (UNG) (ArcticZymes) | |
| RNase Inhibitor, Murine (New England | 0.50 U/μL |
| Biolabs) | |
| Warmstart RTx Reverse Transcriptase (New | 0.30 U/μL |
| England Biolabs) | |
| Bst 2.0 Warmstart DNA Polymerase (New | 0.32 U/μL |
| England Biolabs) | |
| F3-Control Primer (SEQ ID NO: 15) | 0.16 μM |
| F3-Test Primer (SEQ ID NO: 1) | 0.20 μM |
| B3-Control Primer (SEQ ID NO: 16) | 0.16 μM |
| B3-Test Primer (SEQ ID NO: 2) | 0.20 μM |
| FIP-Control Primer (SEQ ID NO: 17) | 1.28 μM |
| FIP-Test Primer (SEQ ID NO: 5) | 1.60 μM |
| BIP-Control Primer (SEQ ID NO: 18) | 1.28 μM |
| BIP-Test Primer (SEQ ID NO: 6) | 1.60 μM |
| LoopF-DIG-Control Primer (SEQ ID NO: 19) | 0.32 μM |
| LoopB-BIOT-Control Primer (SEQ ID NO: | 0.32 μM |
| 20) | |
| LoopF-FAM-Test Primer (SEQ ID NO: 3) | 0.40 μM |
| LoopB-BIOT-Test Primer (SEQ ID NO: 4) | 0.40 μM |
| Deoxynucleotide triphosphates | 1.4 mM each |
| (dATP:dCTP:dGTP) | |
| Deoxynucleotide triphosphates (dTTP:dUTP) | 0.7 mM each |
| Dithiothreitol (DTT) | 5 mM |

These Detect test components included both sample processing and fluid flow controls. First, as indicated in Table 5 above, the Detect test contained primers that targeted the nucleic acid sequences encoding human ribonuclease P (RNase P) (RPP20, POP7). For SARS-CoV-2 negative samples, the detection of RNase P, as indicated by the presence of the Sample Processing Control line on the Reader's lateral flow strip, served as both an extraction control and an internal control. The detection of RNase P demonstrated that 1) the sample collected contained sufficient human genomic material to enable amplification, 2) the heating protocol resulted in successful sample lysis to allow for RNase P detection, and 3) the amplification reaction was successful, indicating that the enzymes involved in amplification were functional and that the heating protocol was correctly executed. This control was present in every Detect test, and its detection was required for the results of the test to be considered valid.

Second, in addition to the RNAse P control, the Detect test also included an additional control line—a Readout Check Control line—on the lateral flow strip of the Reader. This Readout Check Control line indicated that the user correctly dispensed the contents of the Dropper into the Reader's chimney and successfully inserted the Warmer Tube into the Reader, thereby puncturing the tube and initiating wicking of the reaction liquid on the lateral flow strip. The Readout Check Control aided in identifying the underlying root cause of an invalid test result by indicating whether the readout step was performed correctly.

### Positive and Negative SARS-CoV-2 Controls

In addition, in this Example, positive and negative SARS-CoV-2 controls were used to verify that the test components were in good condition and that the test methods were performed correctly. In some cases, tests with both positive and negative controls were performed for each new shipment of Detect tests, each new operator, and in accordance with guidelines or requirements of local, state and/or federal regulations or accrediting organizations on a regular interval as dictated by the laboratory.

The negative control was NATrol™ SARS Associated Coronavirus 2 (SARS-CoV-2) Negative Control (6×0.5 mL), supplied by ZeptoMetrix Corporation (Catalog #: NATSARS(COV2)-NEG), which comprised human A-549 cells at 50,000 cells/mL in a proprietary matrix. The negative control was stored at 2-8° C. The positive control was AccuPlex™ SARS-CoV-2 Control Kit FULL GENOME (6×0.6 mL), supplied by Seracare (Catalog #: 0505-0229). The positive control comprised SARS-CoV-2 genomic material encapsulated in an Accuplex™ recombinant and replication-defective Sindbis virus with full protein coat and lipid bilayer, provided at 3.0×10⁵ genome copies/mL and was stored at 2-8° C.

### Detect Test Steps

To conduct the Detect test, users (also referred to as operators) performed the following steps:

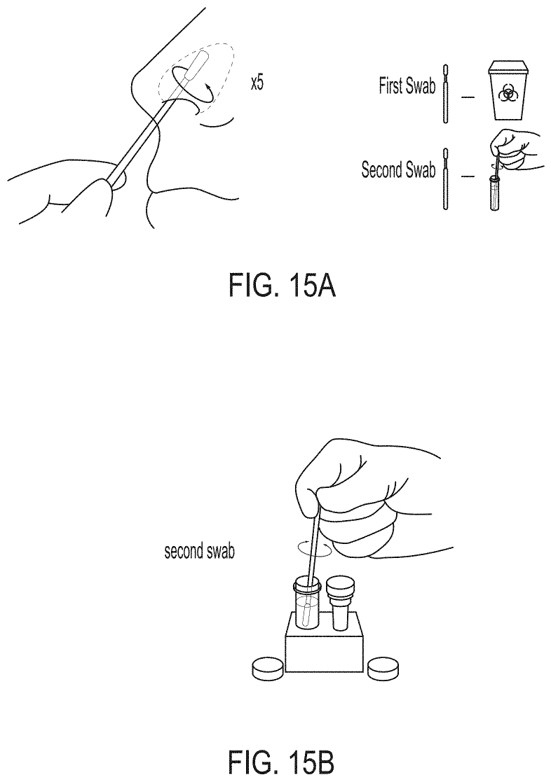

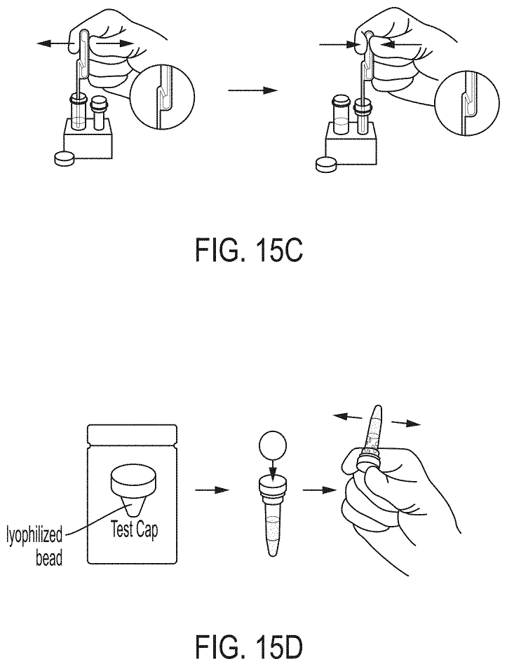

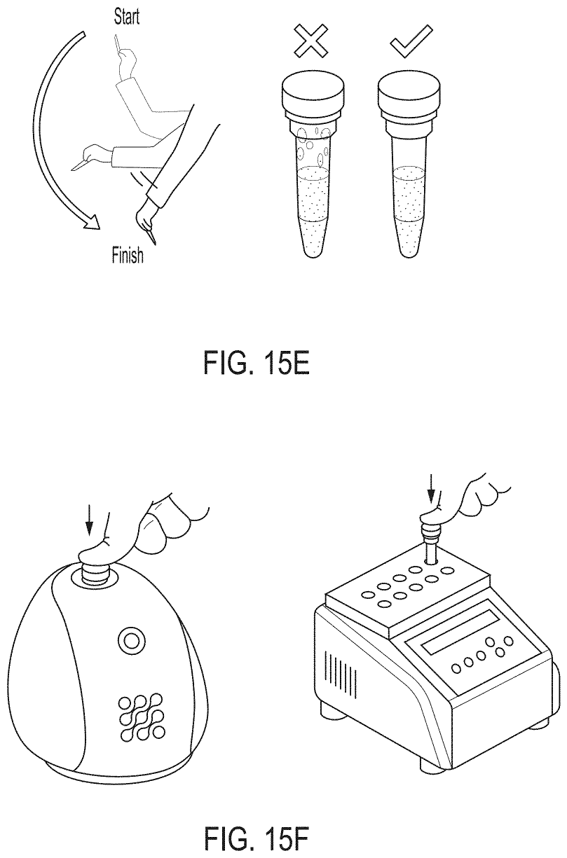

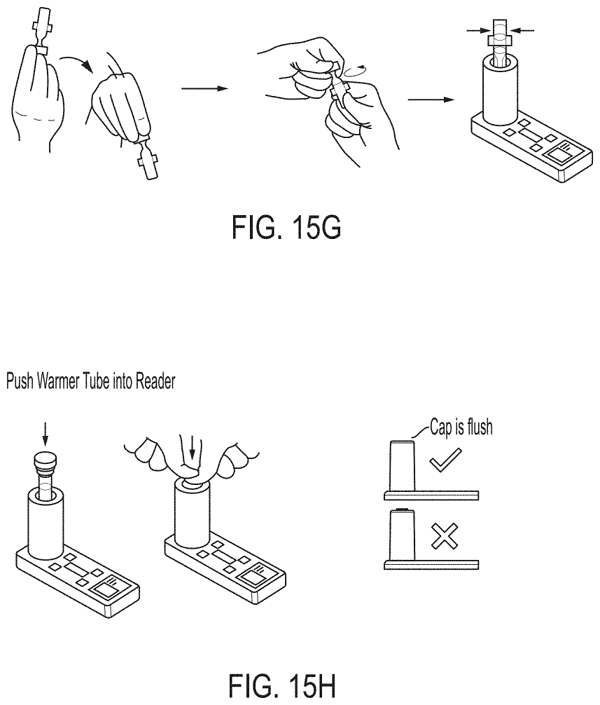

1. Both nostrils of a subject were swabbed using a first nasal Swab, and the first Swab, which was used to clear excess nasal mucus, was discarded (FIG. 15A). Both nostrils of the subject were then swabbed using a second nasal Swab. In some embodiments, the operator was the subject, and the sample was self-collected.

2. The operator inserted the second Swab into the Collection Buffer contained in the Test Kit's “Collection Tube,” releasing the nasal sample into the buffer (FIG. 15B) and then discarding the Swab.

3. The operator used the fixed-volume Transfer Pipette to move 250 μL of liquid (e.g., buffer) from the Collection Tube to the Warmer Tube (i.e., an empty second tube) (FIG. 15C). The volume transferred in this step could be any volume in the range between 200 μL-300 μL without impacting test performance (see Example 2). The Transfer Pipette, with its “overflow bulb,” was designed to make it challenging for an operator to transfer more than 300 μL in a single transfer event.

4. In case a retest was needed, the operator stored the Collection Tube with its remaining sample in the refrigerator. The remnant sample in Collection Buffer could sit for up to four hours—either in refrigerated or room temperature conditions—before being run through the rest of the test procedure without impacting test performance (see Example 2).

5. The operator opened a pouch containing the Test Cap and screwed the Test Cap—a cap with a plastic basket hanging underneath that held a lyophilized reagent bead—onto the Warmer Tube (FIG. 15D). The operator then inverted and shook the tube (FIG. 15D) to resuspend the lyophilized bead's reagents—including all the primers and enzymes needed for the assay—into the liquid. Among the resuspended reagents were an RNase inhibitor, which aided in preventing RNA degradation during any delays between the Test Cap addition and the subsequent heating step. The resuspended reagents also included the assay's amplification enzymes—a reverse transcriptase and a DNA polymerase—which were “warm-start,” meaning that they were inactive at room temperature. Accordingly, the Warmer Tube could sit at room temperature, after the addition of the Test Cap, for up to 2 hours before beginning the heating protocol without impacting performance, giving the operator time to prepare other samples to incubate in a batch (see Example 2).

6. The operator snapped the Warmer Tube downward to move liquid into the bottom of the tube, ensuring that all liquid had pooled at the bottom of the tube (FIG. 15E).

7. The operator then performed one of the following steps (depending on which Detect Warmer the operator was using):
a. If the operator was using the Detect Single-Well Warmer, then the operator inserted the Warmer Tube into the Warmer (FIG. 15F, left), at which point the reaction's pre-programmed heating protocol was automatically initiated. The Warmer's green light blinked for the duration of the heating protocol, which lasted for approximately 55 minutes. After 55 minutes had elapsed, the Warmer beeped twice and the green light stopped blinking to indicate that the reaction had completed and that it was safe to remove the Warmer Tube.
b. If the operator was using the Detect Multi-Well Warmer, then the operator inserted the Warmer Tube(s) into the Warmer (FIG. 15F, right). After the operator inserted all of the Warmer Tubes that were to be run in the batch, the operator applied the Warmer's plastic lid and presses the Warmer's “start” button, thereby initiating the reaction's heating protocol in all 10 wells simultaneously. The heating protocol lasted for approximately 55 minutes. After 55 minutes had elapsed, the Warmer beeped and displayed “Finished” to indicate that the reaction was complete, and the Warmer Tube(s) were removed from the Warmer.

In the first stage of the heating cycle of either Warmer, the liquid temperature was held at 37° C. for 3 minutes, allowing the thermolabile UDG (Uracil DNA glycosylase) enzyme to degrade any contaminating amplicons (that could have resulted from incorrect operation of a previous Detect test) that may have been environmentally introduced into the sample. In the next stage of the heating cycle of either Warmer, the liquid temperature was held at 63.5° C. for 40 minutes. At this elevated temperature, the UDG enzyme was denatured, and viral and human nucleic acids were released and made available for downstream loop-mediated amplification in a multiplexed SARS-CoV-2 and human RNAse P reaction.

Amplification of the SARS-CoV-2 and RNAse P nucleic acids, if present, occurred concurrently in the same tube using the same reagents. The first phase of RT-LAMP involved the conversion of the RNA targets into cDNA by reverse transcriptase. Subsequently, a strand-displacing DNA polymerase initiated DNA synthesis for nucleic acids targeted by the SARS-CoV-2 and human RNAse P primers. Biotin and FAM labels were incorporated into amplified SARS-CoV-2 products, and biotin and DIG labels were incorporated into amplified RNAse P products. After the 40 minutes at elevated temperature, the temperature decreased to 37° C., and the Warmer beeped to indicate that the reaction was complete and that the Warmer Tube could be removed.

7. The operator opened the Test Kit's “Dropper”—an ampoule filled with buffer—into the chimney of the Reader to pre-wet the lateral flow strip housed in the plastic Reader enclosure (FIG. 15G).

8. The operator removed the Warmer Tube from the Warmer and pressed it firmly into the Reader's chimney (FIG. 15H). Upon insertion, the tube's bottom surface was punctured by a blade in the Reader, causing the reaction liquid to wick onto the lateral flow strip. All liquid was contained within the sealed reader—even if the operator inverted or shook the Reader, the reaction liquid remained within the sealed reader.

On the lateral flow strip's sample pad, biotin-labeled RNAse P and SARS-CoV-2 amplicons bound neutravidin-conjugated colored particles and subsequently flowed through the lateral flow strip, where they were captured by immobilized antibodies. SARS-CoV-2 amplicons bound to colored particles aggregated along an anti-FAM antibody coated stripe and caused the formation of a visible line within 10 minutes. Similarly, RNAse P amplicons were captured by anti-DIG antibodies immobilized at a separate location on the lateral flow strip to produce a distinct visible line. In addition, successful wicking of liquid through the strip caused the development of a third line on the lateral flow strip, referred to as the “Readout Check Control.” For the results to be valid, the Readout Check Control line had to be present. A valid negative result also had to show the RNase P Sample Processing Control line.

9. After 10 minutes had elapsed, the operator looked at the lateral flow strip contained in the Reader and interpreted the results (interpretation criteria are described below). The result could be interpreted with no performance decrease at any time between 2 and 60 minutes after insertion of the Warmer Tube into the Reader (see Example 2).

10. In the event of an invalid result, the operator took the stored Collection Tube out of the refrigerator, opened a new Test Kit, and repeated the test procedure, starting with step 3 above, and skipping step 4. Therefore, the operator did not need to collect a new sample in order to perform the retest. The result of this retest was considered final.

### Interpretation of Results

1. Detect Controls—Positive, Negative, and Internal

a. Acceptance Criteria

The assay results were reported via a lateral flow strip, providing a multiplexed readout of spatially distinct colored bands. The result was intended to be read and interpreted directly by the operator. The presence of amplified target RNAs (test or control) in the patient sample resulted in a visible band at the respective location on the lateral flow strip. The intensity of the band(s) was not a factor in interpretation; the appearance of the band, regardless of intensity, was considered a positive result for the Sample Processing Control and/or presence of SARS-CoV-2 RNA.

b. Readout Check Control

If the Readout Check Control band was not visible on the lateral flow strip for a human clinical specimen, no matter which other bands appeared, then the test was considered invalid. The operator then proceeded with a retest.

c. Sample Processing Control—RNase P

If the Readout Check Control band was visible but neither the RNase P Sample Processing Control band nor the SARS-CoV-2 test band were visible on the lateral flow strip for a human clinical specimen, the test result was considered invalid. The operator then proceeded with a retest.

d. Negative Control

Valid negative control tests appeared SARS-CoV-2 negative, with both the Readout Check Control and RNase P Sample Processing Control bands visible.

e. Positive Control

Valid positive control tests appeared SARS-CoV-2 positive, with both the Readout Check Control and SARS-CoV-2 Test bands visible.

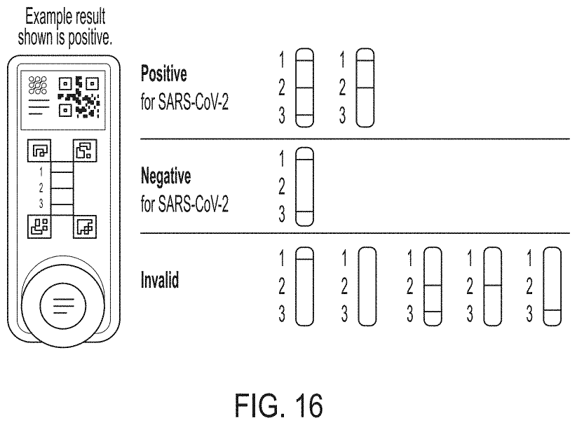

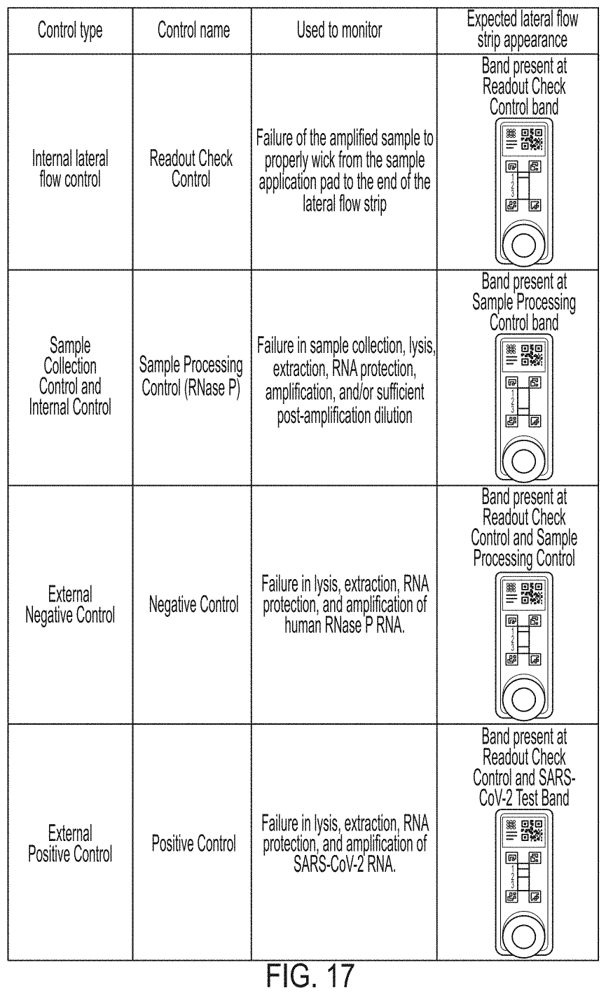

FIGS. 16 and 17 provide an overview of the controls, test bands, and their expected lateral flow appearance. In particular, when there was a SARS-CoV-2 positive result, bands at locations 1, 2, and 3, or at locations 1 and 2, were expected. When there was a SARS-CoV-2 negative result, only bands at locations 1 and 3 were expected. Invalid test results included no bands, only a band any of location 1, 2, or 3, or only bands 2 and 3.

When there was a failure of the amplified sample to properly wick from the sample application pad to the end of the lateral flow strip, only the “Readout Check Control” band was present. When there was a failure in sample collection, lysis, extraction, RNA protection, amplification, and/or sufficient post-amplification dilution, only the “Sample Processing Control” band was present. When there was a failure in lysis, extraction, RNA protection, and/or amplification of human RNase P RNA, both the “Readout Check Control” and “Sample Processing Control” bands were present. When there was a failure in lysis, extraction, RNA protection, and amplification of SARS-CoV-2 RNA, both the “Readout Check Control” and Sars-CoV-2 bands were present. In cases where the pattern of visible lines was classified as invalid, a retest procedure was performed. No numeric test values or individual primer/probe set results were interpreted by any of the operators.

Table 6 shows the actions that were carried out for each possible result.

TABLE 6
Detect Test Results Interpretation Chart

| | | Sample | | | |
|---|---|---|---|---|---|
| Readout | | Processing | | | |
| Check | | Control - | | | |
| Control | SARS-CoV-2 | RNase P | Result | | |
| (Line 1) | (Line 2) | (Line 3) | interpretation | Report | Actions |
| + | + | + or − | SARS-CoV-2 | Positive Test | Report results |
| | | | detected | SARS-CoV-2 | to patient and |
| | | | | | CDC |
| + | − | + | SARS-CoV-2 | Negative Test | Report results |
| | | | not detected | SARS-CoV-2 | to patient and |
| | | | | | CDC |
| + | − | − | Invalid | Invalid | Perform |
| | | | Result | | Retest |
| | | | | | Procedure |
| | + or − | + or − | Invalid | Invalid | Perform |
| | | | Result | | Retest |
| | | | | | Procedure |

### Testing Capabilities; Rapidity of Rapid Diagnostic Test

In some cases, the Detect test was performed in 1 hour and 10 minutes. The steps required to run the Detect test, as described above, included setup, sample collection, Warmer Tube preparation, warming, and reading results.

Table 7 below lists the approximate times required to complete each step for a single Detect test. Using the Multi-Well Warmer, 10 tests were performed per instrument run. It was estimated that in a typical point-of-care setting with a single Multi-Well Warmer, approximately 10 samples could be processed every 1 hour 41 minutes by a single test operator (with another operator overseeing sample collection), and in the course of an 8-hour workday, approximately 50 tests could be performed per day per operator.

TABLE 7
Time Required for Performing the Detect Test

| | |
|---|---|
| | Maximum throughput |
| Time for one test | for one instrument |

| | Time | | Time |
|---|---|---|---|
| Step | (hr:min) | Step | (hr:min) |
| Test setup, Swab | 0:04 | Test setup, Swab | 0:26 |
| collection, Warmer | | collection, Warmer | |
| Tube preparation | | Tube preparation | |
| Warming | 0:55 | Warming | 0:55 |
| Reading Results | 0:11 | Reading Results | 0:20 |
| Total | 1:10 | Total | 1:41 |

### Limit of Detection (LoD)

The Limit of Detection (LoD) was determined using contrived positive samples of heat-inactivated SARS-CoV-2 virus in pooled nasal matrix. The SARS-CoV-2 virus was deposited by the Centers for Disease Control and Prevention and obtained through BEI Resources, NIAID, NIH: SARS-Related Coronavirus 2, Isolate USA-WA1/2020, Heat Inactivated, NR-52286.

a. Contrived Sample Preparation

The pooled nasal matrix was generated by eluting nasal material off of individual frozen and thawed SARS-CoV-2 negative nasal swabs collected using the Swab, as described herein (swabs were confirmed negative using the CDC SARS-CoV-2 RT-PCR assay performed on a second reference swab collected by the same person at the same time). Each swab was eluted by twirling vigorously for 15 seconds into a separate 250 μL aliquot of Collection Buffer, as described herein, and the resulting nasal matrix samples from >10 swabs were combined to make the pool. Pooled nasal matrix was used as diluent to dilute heat-inactivated SARS-CoV-2 virus to the desired concentration, and 50 μL of this spiked pool was pipetted onto an unused Swab and subjected to the Detect test workflow as described herein.

b. Preliminary LoD Determination

Initially, based on past studies conducted during product development, it was correctly predicted that starting the dilution series for the Final LoD Determination at 16,500 copies per swab would facilitate an efficient study.

c. Final LoD Determination

The Detect test was run on 20 replicates (produced according to the Contrived Sample Preparation procedure described above) over a series of 2-fold dilutions, starting with an initial concentration of 16,500 copies per swab, until the final LoD was established. The Detect test's final LoD was determined to be 8,250 copies/swab. Results are shown in Table 8, below.

TABLE 8
Limit of Detection for the Detect Test
Using Heat-Inactivated SARS-CoV-2

| | Concentration in | | |
|---|---|---|---|
| Viral Load | Reaction if Complete | | |
| (genomic | Transfer from Swab | Detection | |
| copies/swab) | (genomic copies/μL) | Rate | % Detected |
| 4,125 | 2.5 | 8/20 | 40% |
| 8,250 | 5 | 20/20 | 100% |
| 16,500 | 10 | 20/20 | 100% |

### Inclusivity of SARS-CoV-2 Orflab Primer Set for SARS-CoV-2

In silico inclusivity analysis of the SARS-CoV-2 orf1ab primer set described herein was performed based on the set of all 170,190 SARS-CoV-2 genomes available in the GISAID EpiCoV database (www.gisaid.org) on Nov. 3, 2020. Genomes annotated as originating from a human host were extracted and analyzed using the Nextclade pipeline (clades.nextstrain.org; developed specifically for SARS-CoV-2 genome sequences) with default parameters. Nextclade was used to a) remove low-quality genome sequences with its Quality Control process, b) align the resulting 114,757 high-quality sequences to the SARS-CoV-2 reference genome, and c) call mutations and indels in these 114,757 sequences relative to the SARS-CoV-2 reference genome. All incidences of mutations and indels in primer-binding positions were then computed.

97.4% of genomes contained no mismatches with any of the 6 primers (SEQ ID NOs: 1-6). Based on an in silico analysis, at least 99% of all 114,757 genomes were expected to be robustly detected by the primer set, with no more than 2 primer-binding sites containing no more than a single mismatch, and no primer-binding sites containing any indels or critical-region mismatches as defined by the Primer Explorer v5 Manual 1 (https://primerexplorer.jp/e/v5_manual/pdf/PrimerExplorerV5_Manual_1.pdf, pg. 1), published by LAMP inventor Eiken Chemical Corporation (mismatches in 6 terminal nucleotides at the 3′ termini of any of the 6 primers, as well as at the 5′ termini of FIP and BIP).

### Inclusivity of SARS-CoV-2 Orflab Primer Set for Emerging SARS-CoV-2 Variants (B.1.1.7 Lineage and B.1.351 Lineage)

In addition, in silico inclusivity analysis of the SARS-CoV-2 orf1ab primer set (SEQ ID NOs: 1-6) was performed with the two emerging SARS-CoV-2 variants currently listed by the U.S. Centers for Disease Control and Prevention (CDC): B.1.1.7 lineage (a.k.a. 20B/501Y.V1 Variant of Concern (VOC) 202012/01) and B.1.351 lineage (a.k.a. 20C/501Y.V2).

a. B.1.1.7 Lineage

None of the 23 B.1.1.7 lineage-defining mutations listed in the COVID-19 Genomics Consortium UK's December 2020 Report, entitled Preliminary genomic characterization of an emergent SARS-CoV-2 lineage in the UK defined by a novel set of spike mutations, occurred within the orf1ab region targeted by the SARS-CoV-2 primer set described herein (nucleotide positions 2245-2441 in the reference SARS-CoV-2 genome [Wuhan-Hu-1, NCBI Accession NC_045512.2]). Inclusivity with the B.1.1.7 lineage was further verified with a global alignment between the targeted region (+20 nucleotides flanking either side) of the canonical B.1.1.7 genome (GISAID accession EPI_ISL_601443, Public Health England's December 2020 Report entitled Investigation of novel SARS-COV-2 Variant: Variant of Concern 202012/01) and the NCBI SARS-CoV-2 Reference Sequence genome (Wuhan-Hu-1, NCBI Accession NC_045512.2) the primer set was designed upon. The 2 genomes were found to match perfectly over this region. Accordingly, the SARS-CoV-2 orf1ab primer set described herein (SEQ ID NOs: 1-6) is inclusive of the 23 known B.1.1.7 lineage-defining mutations.

b. B.1.351 Lineage

All 9 B.1.351 lineage-defining mutations identified by Tegally et al. (2020) [Emergence and rapid spread of a new severe acute respiratory syndrome-related coronavirus 2 (SARS-CoV-2) lineage with multiple spike mutations in South Africa, https://doi.org/10.1101/2020.12.21.20248640] occurred within the S gene, and so did not affect the orf1ab region targeted by Detect's SARS-CoV-2 primer set. Inclusivity with the B.1.351 lineage was further verified with a global alignment between the targeted region (+20 nucleotides flanking either side) of the original B.1.351 genome (GISAID accession EPI_ISL_712081, collection date 2020 Oct. 8) and the NCBI SARS-CoV-2 Reference Sequence genome (Wuhan-Hu-1, NCBI Accession NC_045512.2) the primer set was designed upon. The 2 genomes were found to match perfectly over this region. Accordingly, the SARS-CoV-2 orf1ab primer set described herein (SEQ ID NOs: 1-6) was found to be inclusive of the 9 B.1.351 lineage-defining mutations.

### Cross-Reactivity Analysis

a. In Silico Cross-Reactivity Analysis

In silico cross-reactivity analysis of the primer sequences of both the SARS-CoV-2 orf1ab primer set and the human RNase P (RPP20, POP7) (sample processing control) primer set was performed by querying them in a BLASTn search against a local database consisting of reference/representative genome sequences of the specifically named respiratory microorganisms listed in Table 9, downloaded from NCBI. The BLASTn parameters were: Word Size=6; Expect Threshold=1,000; Gap Open=5; Gap Extend=2; Reward=1; Penalty=−3. Percentage homology was computed based on the number of matching positions in an alignment relative to the full length of the aligned primer. Table 9 details all detected instances of ≥80% homology between a primer and respiratory microorganism genome.

≥80% homology was only apparent for the SARS-CoV-2 orf1ab B3 primer (with Candida albicans) and the Human Rnase P (internal positive control) F3 primer (with Mycobacterium tuberculosis (2 sites), Pneumocystis jirovecii (PJP) and Pseudomonas aeruginosa). Thus, only 1 primer from each set displayed ≥80% homology with any of the respiratory microorganisms investigated, and none of the microorganisms displayed ≥80% homology with more than 1 of the 12 primers. Further, none of the 4 labelled primers (LoopF and LoopB from each set) required for detection of the amplified nucleic acid target on the lateral flow strip showed ≥80% homology with any of the listed respiratory microorganism genomes. Therefore, the in silico analysis identified no potential unintended cross-reactivity of the Detect test with the listed respiratory pathogens, including other coronaviruses.

TABLE 9
In Silico Cross-reactivity Analysis of Detect Primer Sequences

| | | | Human Rnase P |
|---|---|---|---|
| | | SARS-CoV-2 | Sample Processing |
| Organism | NCBI Accession | primer set | Control primer set |
| Human coronavirus | NC_002645.1 | no alignment found | no alignment found |
| 229E | | | |
| Human coronavirus | NC_006213.1 | no alignment found | no alignment found |
| OC43 | | | |
| Human coronavirus | NC_006577.2 | no alignment found | no alignment found |
| HKU1 | | | |
| Human coronavirus | NC_005831.2 | no alignment found | no alignment found |
| NL63 | | | |
| MERS-CoV | NC_038294.1 | no alignment found | no alignment found |
| SARS-CoV | NC_004718.3 | no alignment found | no alignment found |
| Adenovirus (e.g., C1 | AC_000017.1 | no alignment found | no alignment found |
| Ad. 71) | | | |
| Human | NC_039199.1 | no alignment found | no alignment found |
| Metapneumovirus | | | |
| (hMPV) | | | |
| Parainfluenza virus 1 | NC_003461.1 | no alignment found | no alignment found |
| Parainfluenza virus 2 | NC_003443.1 | no alignment found | no alignment found |
| Parainfluenza virus 3 | NC_001796.2 | no alignment found | no alignment found |
| Parainfluenza virus 4 | NC_021928.1 | no alignment found | no alignment found |
| Influenza A | GCF_000865085.1 | no alignment found | no alignment found |
| Influenza B | GCF_000820495.2 | no alignment found | no alignment found |
| Enterovirus (e.g., | NC_038308.1 | no alignment found | no alignment found |
| EV68) | | | |
| Respiratory syncytial | NC_001803.1 | no alignment found | no alignment found |
| virus | | | |
| Rhino virus A | NC_038311.1 | no alignment found | no alignment found |
| Rhino virus B | NC_038312.1 | no alignment found | no alignment found |
| Rhino virus C | NC_009996.1 | no alignment found | no alignment found |
| Chlamydia | NC_005043.1 | no alignment found | no alignment found |
| pneumoniae | | | |
| Haemophilus | NZ_LN831035.1 | no alignment found | no alignment found |
| influenzae | | | |
| Legionella | NZ_LR 1343 80.1 | no alignment found | no alignment found |
| pneumophila | | | |
| Mycobacterium | NC_000962.3 | no alignment found | F3 primer only, 82% |
| tuberculosis | | | |
| Streptococcus | NZ_LN831051.1 | no alignment found | no alignment found |
| pneumoniae | | | |
| Streptococcus | NZ_LN831034.1 | no alignment found | no alignment found |
| pyogenes | | | |
| Bordetella pertussis | NC_018518.1 | no alignment found | no alignment found |
| Mycoplasma | NZ_CP010546.1 | no alignment found | no alignment found |
| pneumoniae | | | |
| Pneumocystis | GCF_001477535.1 | no alignment found | F3 primer only, 88% |
| jirovecii (PJP) | | | |
| Candida albicans | GCF_000182965.3 | no alignment found | no alignment found |
| Pseudomonas | NC_002516.2 | no alignment found | F3 primer only, 82% |
| aeruginosa | | | |
| Staphylococcus | GCF_000007645.1 | no alignment found | no alignment found |
| epidermidis | | | |
| Staphylococcus | NC_013715.1 | no alignment found | no alignment found |
| salivarius | | | |

b. In Vitro Cross-Reactivity Analysis

The Detect test's cross-reactivity with certain organisms (e.g., closely related pathogens, common disease agents, and normal and pathogenic flora) was further tested by spiking the organism or genomic material from the organism directly into triplicate reactions at the concentrations listed in Table 10 below. The Detect test showed no interaction with any of the 31 organisms tested.

The Detect test was also tested repeatedly with pooled human nasal matrix as the negative contrived samples in all reported flex studies (115 replicates in all) and showed no cross-reactivity.

TABLE 10
In vitro Cross-reactivity Testing

| | | Concentration | | Human | Cross- |
|---|---|---|---|---|---|
| | | tested | SARS-CoV-2 | control gene | reactivity |
| | | (in final | # Detected/ | # Detected/ | with Detect |
| Organism | Target | reaction) | # tested | # tested | test |
| Human | Synthetic | 1.00E+06 | 0/3 | 0/3 | No |
| coronavirus | RNA | copies/mL | | | |
| 229E | | | | | |
| Human | Synthetic | 1.00E+06 | 0/3 | 0/3 | No |
| coronavirus | RNA | copies/mL | | | |
| OC43 | | | | | |
| Human | Synthetic | 1.00E+06 | 0/3 | 0/3 | No |
| coronavirus | RNA | copies/mL | | | |
| HKU1 | | | | | |
| Human | Virus | 4.00E+04 | 0/3 | 0/3 | No |
| coronavirus | | TCID50/mL | | | |
| NL63 | | | | | |
| MERS- | Synthetic | 1.00E+06 | 0/3 | 0/3 | No |
| coronavirus | RNA | copies/mL | | | |
| SARS- | Synthetic | 1.00E+06 | 0/3 | 0/3 | No |
| coronavirus | RNA | copies/mL | | | |
| Adenovirus | Virus | 1.00E+05 | 0/3 | 1/3 | No |
| (Adenoid 71) | | TCID50/mL | | | |
| Human | Virus | 1.00E+05 | 0/3 | 0/3 | No |
| Metapneumovirus | | TCID50/mL | | | |
| (hMPV) | | | | | |
| Parainfluenza | Synthetic | 1.00E+06 | 0/3 | 0/3 | No |
| virus 1 | RNA | copies/mL | | | |
| Parainfluenza | Virus | 1.00E+05 | 0/3 | 0/3 | No |
| virus 2 | | TCID50/mL | | | |
| Parainfluenza | Virus | 1.00E+05 | 0/3 | 0/3 | No |
| virus 3 | | TCID50/mL | | | |
| Parainfluenza | Synthetic | 1.00E+06 | 0/3 | 0/3 | No |
| virus 4 | RNA | copies/mL | | | |
| Influenza A | Synthetic | 1.00E+06 | 0/3 | 0/3 | No |
| H1N1 | RNA | copies/mL | | | |
| Influenza A | Synthetic | 1.00E+06 | 0/3 | 0/3 | No |
| H3N3 | RNA | copies/mL | | | |
| Influenza B | Synthetic | 1.00E+06 | 0/3 | 0/3 | No |
| | RNA | copies/mL | | | |
| Enterovirus 68 | Synthetic | 1.00E+06 | 0/3 | 0/3 | No |
| | RNA | copies/mL | | | |
| Respiratory | Virus | 1.00E+05 | 0/3 | 3/3‡ | No |
| syncytial virus | | PFU/mL | | | |
| (Subgroup A) | | | | | |
| Rhinovirus 89 | Synthetic | 1.00E+06 | 0/3 | 0/3 | No |
| | RNA | copies/mL | | | |
| Chlamydia | Bacteria | 1.00E+06 | 0/3 | 3/3‡ | No |
| pneumoniae | | IFU/mL | | | |
| Haemophilus | Bacteria | 1.00E+06 | 0/3 | 0/3 | No |
| influenzae | | CFU/mL | | | |
| Legionella | Bacteria | 1.00E+06 | 0/3 | 0/3 | No |
| pneumophila | | CFU/mL | | | |
| Mycobacterium | Genomic | 1.00E+06 | 0/3 | 0/3 | No |
| tuberculosis | DNA | copies/mL | | | |
| Streptococcus | Genomic | 1.00E+06 | 0/9 | 1/9* | No |
| pneumoniae | DNA | copies/mL | | | |
| Streptococcus | Genomic | 1.00E+06 | 0/3 | 0/3 | No |
| pyogenes | DNA | copies/mL | | | |
| Bordetella | Genomic | 1.00E+06 | 0/3 | 0/3 | No |
| pertussis | DNA | copies/mL | | | |
| Mycoplasma | Genomic | 1.00E+06 | 0/3 | 0/3 | No |
| pneumoniae | DNA | copies/mL | | | |
| Pneumocystis | Yeast | 1.00E+06 | 0/3 | 0/3 | No |
| jirovecii (PJP)- | | CFU/mL | | | |
| S. cerevisiae** | | | | | |
| Candida albicans | Yeast | 1.00E+06 | 0/3 | 0/3 | No |
| | | CFU/mL | | | |
| Pseudomonas | Genomic | 1.00E+06 | 0/3 | 0/3 | No |
| aeruginosa | DNA | copies/mL | | | |
| Staphylococcus | Genomic | 1.00E+06 | 0/3 | 0/3 | No |
| epidermis | DNA | copies/mL | | | |
| Streptococcus | Genomic | 1.26E+02 | 0/9 | 1/9* | No |
| salivarius | DNA | ng/mL | | | |

‡Human control gene was detected due to the viral or bacterial stock available containing cell culture lysate.
*Initial cross-reactivity testing with synthetic RNA showed RNase P detection in one replicate, but follow-up testing showed no detection. The source of the original one replicate detection is believed to have been environmental contamination.
**Due to limited pathogen availability, cross-reactivity was tested with a recombinant version of S. cerevisiae containing genomic material from PJP.

c. Microbial Interference Studies

Microbial interference studies were not performed for Mycobacterium tuberculosis, Pneumocystis jirovecii (PJP), Candida albicans, or Pseudomonas aeruginosa since in each case the primer alignment found corresponded to a single primer, and in vitro testing with genomic material or the pathogen itself showed no evidence of cross-reactivity with these organisms.

d. Endogenous Interference Substances Studies

Common endogenous and exogenous substances that may be present in clinical nasal swab samples were tested for interference with the Detect test. Each potentially interfering substance was spiked into both negative pooled nasal matrix and contrived positive pooled nasal matrix spiked with heat-inactivated SARS-CoV-2 virus at 2× LoD. From these pools, triplicate swabs were tested using the Detect test. The interfering substances and their concentrations are listed in the Table 11 below. The results show that the Detect test was robust to a wide range of potentially interfering substances, with the exception of biotin at concentrations above 0.13 μg/mL.

TABLE 11
Detect Interfering Substances Study

| | Final | | Negative | Positive |
|---|---|---|---|---|
| | Concentration | | Samples | Samples |
| Interfering | in Nasal | | #Negative/ | # Positive/ |
| substance | Matrix Pool | | # tested | # tested |
| Rhinocort Allergy | 15% | v/v | 3/3 | 3/3 |
| Afrin Nasal | 15% | v/v | 3/3 | 3/3 |
| Congestion Relief | | | | |
| Spray | | | | |
| Zicam Cold Remedy | 15% | v/v | 3/3 | 3/3 |
| Nasal Spray | | | | |
| Chloraseptic Sore | 15% | v/v | 3/3 | 3/3 |
| Throat Spray | | | | |
| Flonase Allergy | 15% | v/v | 3/3 | 3/3 |
| Relief Nasal Spray | | | | |
| Mupirocin | 5 | mg/mL | 3/3 | 3/3 |
| Neo-Synephrine | 15% | v/v | 3/3 | 3/3 |
| Nasal Saline Spray | 15% | v/v | 3/3 | 3/3 |
| Tobramycin | 600 | μg/mL | 3/3 | 3/3 |
| Fresh whole blood | 10% | v/v | 3/3 | 3/3 |
| Biotin | 3.5 | μg/mL | 0/3* | 0/3* |
| | 1.17 | μg/mL | 3/3 | 2/3* |
| | 0.39 | μg/mL | 2/3* | 3/3 |
| | 0.13 | μg/mL | 3/3 | 3/3 |
| Dexamethasone | 15% | v/v | 3/3 | 3/3 |
| Flunisolide | 15% | v/v | 3/3 | 3/3 |
| Mucin | 1 | mg/mL | 3/3 | 3/3 |
| Triamcinolone | 15% | v/v | 3/3 | 3/3 |
| Mometasone nasal | 1 | mg/mL | 3/3 | 3/3 |
| spray | | | | |

*Undetected replicates were all invalid.

### Clinical Evaluation: Studies to Support Point-of-Care Indication

a. Clinical Evaluation

The clinical evaluation was a prospective, multi-center study (CS1206-01). Four (4) geographically diverse locations were used. Testing in the reference laboratory was performed by trained laboratory personnel. Testing at the point-of-care (POC) sites was performed by non-laboratory health professionals who were representative of typical intended use operators (e.g., nurses, physician assistants, medical assistants, etc.).

A qualified research professional was designated as the Investigator at each site, with the responsibility for oversight of the study in accordance with Good Clinical Practice and regulatory requirements. The protocol and subject informed consent were reviewed by an Institutional Review Board (IRB), and written IRB approval was issued prior to enrollment of subjects into the study at that site.

A subject's participation in this study consisted of a single visit. Following completion of the informed consent process and a review of Inclusion/Exclusion criteria (see below) to determine eligibility, each subject received a unique study identification number. Subjects were asked about their relevant medical history, including the presence or absence of COVID-19 signs and symptoms on the day of the visit and within the last 14 days (including date of onset of symptoms) and current medications taken. Subject demographics, including age, sex, race, and ethnicity, education level, and socioeconomic background, were also collected.

Subjects self-collected or collected from their child two (2) anterior nares (nasal) swabs according to the Detect test kit self-collection instructions. Any complications in obtaining the specimens were documented. Once the subject self-collected two swabs, the intended use operator performed the Detect test using the second nasal swab. The first nasal swab collected was prepared in viral transport media, as specified in the comparator assay's instructions-for-use, and sent to a reference lab for testing. It was decided not to randomize the order of the comparator and Detect test swabs, in order to allow the Detect test to be run exactly according to its intended procedure (from the second swab). The subject was not informed of the results from the Detect test; however, they could choose to contact the study site for the results of their reference test. See Table 12 for the clinical evaluation study protocol, which is identified throughout this Example as study CS-1206-01. In Table 12, the diagnostic test is referred to as “COVID DETECT.”

TABLE 12
CS-1206-01 Protocol Summary

| Protocol | A Multicenter Study Conducted to Evaluate the Performance of the Homodeus | |
|---|---|---|
| Name | COVID DETECT at Point of Care Testing Sites | |
| Protocol | CS-1206-01 | |
| Number | | |
| Intended | The COVID DETECT assay is an end-point nucleic acid amplification test | |
| Use | intended for the presumptive qualitative detection of RNA from SARS-CoV-2 | |
| | in human anterior nares (nasal) specimens. COVID DETECT is intended for | |
| | use on samples from individuals suspected of COVID-19 by their healthcare | |
| | provider or for screening of individuals without symptoms or other reasons to | |
| | suspect COVID-19 infection. Testing is limited to laboratories certified under | |
| | the Clinical Laboratory Improvement Amendments of 1988 (CLIA), 42 U.S.C. | |
| | §263a, that meet the requirements to perform high, moderate, or waived | |
| | complexity tests. COVID DETECT is authorized for use at the Point of Care | |
| | (POC), i.e., in patient care settings operating under a CLIA Certificate | |
| | of Waiver, Certificate of Compliance, or Certificate of Accreditation. | |
| | Results are for the identification of SARS-CoV-2 RNA. The SARS-CoV-2 | |
| | RNA is generally detectable in anterior nares (nasal) specimens during the acute | |
| | phase of infection. Positive results are indicative of the presence of SARS-CoV- | |
| | 2 RNA; clinical correlation with patient history and other diagnostic | |
| | information is necessary to determine patient infection status. Positive results | |
| | do not rule out bacterial infection or co-infection with other viruses. The agent | |
| | detected may not be the definite cause of disease. Laboratories within the | |
| | United States and its territories are required to report all test results to the | |
| | appropriate public health authorities. | |
| | Negative results do not preclude SARS-CoV-2 infection and should not be used | |
| | as the sole basis for patient management decisions. Negative results must be | |
| | combined with clinical observations, patient history, and epidemiological | |
| | information. | |
| | Use of COVID DETECT in a general, asymptomatic screening population is | |
| | intended to be used as part of an infection control plan, that may include | |
| | additional preventive measures, such as a predefined serial testing plan or | |
| | directed testing of high-risk individuals. Negative results should be considered | |
| | presumptive and do not preclude current or future infection obtained through | |
| | community transmission or other exposures. Negative results must be | |
| | considered in the context of an individual's recent exposures, history, presence | |
| | of clinical signs and symptoms consistent with COVID-19. | |
| | COVID DETECT is intended for use by untrained users. | |
| Number of | Approximately four-hundred (400) subjects will be enrolled in this study. Thirty | |
| Subjects | (30) SARS-CoV-2 positive, and at least thirty (30) samples negative for SARS- | |
| | CoV-2. Depending on the prevalence of SARS-CoV-2, it will be necessary to | |
| | collect a significantly higher number of total samples to obtain the required | |
| | number of positives. Banked or contrived specimens may be used to | |
| | supplement the data set. | |
| | The target enrollment is as follows: | |
| | Age (years) | Target enrollment |
| Target Age | <14 | ~20% |
| Group | 14-24 | ~10-15% |
| Percentages | 24-64 | ~30-35% |
| | >65 | ~35% |

| | |
|---|---|
| Number of | Approximately four (4) Point of Care locations (e.g., physician office |
| Sites | laboratories, urgent care centers, emergency departments, and outpatient clinics |
| | including drive through testing sites). |
| Study | The study will be conducted during the 2020 COVID-19 (SARS-CoV-2) |
| Duration | pandemic in the United States. The study is anticipated to run for approximately |
| | two (2) months. If sufficient fresh specimens are not collected during this time, |
| | the study may be continued as necessary. |
| Objectives | The objective of this study is to evaluate the performance of the COVID |
| of the | DETECT in detecting SARS-CoV-2 in fresh anterior nares swab specimens |
| Study | from patients with signs and symptoms of Covid-19 illness as compared with an |
| | FDA EUA approved comparative method. The study results are intended to |
| | support an EUA submission to the FDA. |
| Subject | Inclusion Criteria |
| Eligibility | 1. The subject may be of any age and either sex. |
| Criteria | 2. Written informed consent must be obtained prior to study enrollment. |
| | a. A subject who is eighteen (18) years or older must be willing to give written |
| | informed consent and must agree to comply with study procedures. |
| | b. The Legal Guardian or Legal Authorized Representative of a subject who |
| | is under the age of eighteen (18) must give written informed consent and |
| | agree to comply with study procedures. Active assent should be obtained |
| | from children of appropriate intellectual age (as defined by the IRB). |
| | In addition to the following: |
| | Group A: Symptomatic Subjects |
| | Preliminary assessment of the subject by the Investigator/designee should be |
| | suggestive of COVID-19 at the time of the study visit. The subject must present |
| | as symptomatic, meaning they have exhibited one or more of the following |
| | signs and symptoms for eligibility: fever, cough, shortness of breath, difficulty |
| | breathing, muscle pain, headache, sore throat, chills, repeated shaking with |
| | chills, new loss of taste or smell, nausea, vomiting or diarrhea. The onset of |
| | these symptoms will be recorded. |
| | or |
| | The subject must have a documented SARS-CoV-2 PCR test in the past 48 |
| | hours. |
| | Group B: Asymptomatic Subjects |
| | (Approximately 20 positive subjects will be enrolled for Group B) |
| | The subject is not exhibiting signs and symptoms of COVID-19. |
| | Exclusion Criteria |
| | 1. The subject underwent a nasal wash/aspirate as part of standard of care |
| | testing during this study visit. |
| | 2. The subject is currently receiving or has received within the past thirty (30) |
| | days of the study visit an experimental biologic, drug, or device including either |
| | treatment or therapy including COVID-19 vaccine. |
| | 3. The subject has previously participated in this research study (CS-1206-01) |
| | 4. The subject is currently undergoing chemotherapy treatment with |
| | documented low platelet and low white blood cell counts. |
| Study | Patients presenting to their health care professionals or drive through testing |
| Procedures | sites with signs and symptoms of COVID-19 will be approached for potential |
| | participation in the study. |
| | A subject's participation in this study will consist of a single visit. Following |
| | completion of the informed consent process and a review of |
| | Inclusion/Exclusion criteria to determine eligibility, each subject will receive a |
| | unique study identification number. |
| | Subjects will be asked relevant medical history questions, including inquiries |
| | regarding the presence of COVID-19 signs and symptoms. Relevant over the |
| | counter medications and herbal remedies taken within the last seventy-two (72) |
| | hours will be recorded. Prescription medications relating to COVID-19 taken |
| | within the past fourteen (14) days of enrollment will also be recorded. Subject |
| | demographics including age, sex, race, and ethnicity will also be recorded. |
| | The subject will be given the COVID DETECT self-collection instructions and |
| | the study staff will instruct the subject to collect two anterior nares swabs on |
| | themselves or their child. The COVID DETECT will be performed at POC sites |
| | by untrained intended use operators (e.g., nurses, physician assistants, medical |
| | assistants, etc.). Each site will have a minimum of 1 intended use operator who |
| | will perform testing under this protocol. |
| | Each operator must perform the COVID DETECT from start to finish. |
| | Reference method specimens will be shipped within twenty-four (24) hours of |
| | collection to the central reference laboratory. |
| Laboratory | EUA authorized SARS-CoV-2 molecular assay (for the COVID-19 |
| Reference | Coronavirus) |
| Method | |
| Residual | Residual transport media will be stored at ≤−70° C. and be shipped to Homodeus |
| Specimens | for possible future testing. |
| Sample | Self- Collected Anterior Nares Swabs |
| Type(s) | |
| Data | Positive Percent Agreement (PPA) and Negative Percent Agreement (NPA) will |
| Analysis | be estimated, along with their associated 2-sided Wilson Score 95% Confidence |
| | Intervals, for the Homodeus COVID DETECT Test results as compared with |
| | the reference test. |

TABLE 13
2 × 2 table for calculation of PPA
(sensitivity) and NPA (specificity)
Reference Test

| | | Positive | Negative | Row Totals |
|---|---|---|---|---|
| COVID | Positive | a | b | |
| DETECT test | Negative | c | d | |
| | Column Totals | | | |

Sensitivity (PPA) (%) = (a/(a + c)) × 100
Specificity (NPA) (%) = (d/(d + b)) × 100

i. Sites and Test Users (Operators)

Four (4) point-of-care locations enrolled study participants, according to the clinical evaluation protocol for study CS-1206-01. Information about these sites is listed in Table 14.

TABLE 14
CS-1206-01 Clinical Study Sites

| Site | Institution | Principal | Enrollment | CLIA |
|---|---|---|---|---|
| Code | Name | Investigator | Address | Certificate |
| ADP | Advanced | Christina Ulen, | 100 East Street SE | Certificate of |
| | Pediatrics | MD | Suite #301&302 | Waiver |
| | Research, LLC | | Vienna, VA 22180 | |
| PPM | UPenn COVID | Benjamin | 51 N. 39th Street | *Certificate of |
| | Clinic | Abella | Andrew Mutch | Accreditation |
| | | | Building Room 313 | |
| | | | Philadelphia, PA | |
| | | | 19104 | |
| SFR | South Florida | Giralt Yanez | 7911 NW 72 Avenue | Certificate of |
| | Research | | Suite 215B | Waiver |
| | Organization | | Medley, FL 33166 | |
| VHP | Village Health | Sander Gothard | 7300 Eldorado | *Certificate of |
| | Partners - Covid | | Parkway Suite 200 | Provider |
| | Clinic | | McKinney, TX | Performed |
| | | | 75070 | Microscopy |

*Intended use operators at this site were representative of those in a CLIA Waived setting.

Intended use operators who performed subject tests are summarized in Table 15. The data reported in this table—and all other tables thereafter—is inclusive only of data that supported the Detect test's proposed intended use for symptomatic subjects aged 14 and older.

TABLE 15
CS-1206-01 Intended Use Operators/Background

| Site Code | Operator Initials | Background |
|---|---|---|
| ADP | K-D | Nursing Assistant, |
| | | Administrator/Office |
| PPM | PLC | Medical Assistant |
| SFR | ADV | Medical Assistant |
| VHP | BNB | Medical Assistant |
| | MEB | Medical Assistant |

ii. Comparator Method

One (1) laboratory was used during CS-1206-01:
TriCore Reference Laboratories
1001 Woodward Place NE
Albuquerque, N. Mex. 87102

Two reference tests were used during the study: the CDC 2019 Novel Coronavirus (2019-nCoV) Real-Time Reverse Transcriptase (RT)-PCR Diagnostic Panel and the Roche Cobas® SARS-CoV-2 Test. All samples were run through both the CDC assay and the cobas test. Each reference sample was run first through the CDC assay and then through the cobas test using leftover specimens from the original reference swab's transport medium. On all samples, the specimens were stored, collected and run in compliance with both the CDC assay's and the cobas test's instructions for use (IFU). It was of particular interest to assess the Detect test's performance, with its orf1ab target, to that of the CDC assay, which targets the N1 and N2 genes, and to that of the cobas test, which targets orf1ab and the E gene. All performance tables below are presented twice: one evaluating the Detect test's performance with CDC assay as comparator, and the second evaluating the Detect test's performance with the cobas test as comparator.

iii. Clinical Samples

Clinical samples were obtained from subjects who met the following inclusion/exclusion criteria to be enrolled in this study:

Inclusion Criteria
1. The subject may be of any age and either sex.
2. Written informed consent must be obtained prior to study enrollment.
   a. A subject who is eighteen (18) years or older must be willing to give written informed consent and must agree to comply with study procedures.
   b. The Legal Guardian or Legal Authorized Representative of a subject who is under the age of eighteen (18) must give written informed consent and agree to comply with study procedures. Active assent should be obtained from children of appropriate intellectual age (as defined by the IRB).
3. Group A: Symptomatic Subjects
   Preliminary assessment of the subject by the Investigator/designee should be suggestive of COVID-19 at the time of the study visit. The subject must present as symptomatic, meaning they have exhibited one or more of the following signs and symptoms for eligibility: fever, cough, shortness of breath, difficulty breathing, muscle pain, headache, sore throat, chills, repeated shaking with chills, new loss of taste or smell, nausea, vomiting or diarrhea. The onset of these symptoms will be recorded.
or
   The subject must have a documented SARS-CoV-2 positive PCR test in the past 48 hours.
4. Group B: Asymptomatic Subjects
   The subject is not exhibiting signs and symptoms of COVID-19.

Exclusion Criteria
1. The subject underwent a nasal wash/aspirate as part of standard of care testing during this study visit.
2. The subject is currently receiving or has received within the past thirty (30) days of the study visit an experimental biologic, drug, or device including either treatment or therapy.
3. The subject has previously participated in this research study (CS-1206-01).
4. The subject is currently undergoing chemotherapy treatment with documented low platelet and low white blood cell counts.

The clinical study was started on Dec. 7, 2020. For this study, a total of one hundred and seventy-eight (178) subjects were enrolled from Dec. 7, 2020 to Jan. 4, 2021.

The data presented in the subsequent analyses and performance tables include data from the testing only of symptomatic subjects greater than 14 years of age.

iv. Performance Analysis

The Detect test's performance compared to the CDC assay and cobas test are presented below in Tables 14 and 15, respectively. The Detect test was demonstrated to have 95.6% PPA (43/45) and 100.0% NPA (62/62) compared to the CDC assay, and 97.7% PPA (43/44) and 100.0% NPA (63/63) when compared to the cobas test.

TABLE 16
Detect Test Performance Against the CDC 2019
Novel Coronavirus (2019-nCoV) Real-Time Reverse
Transcriptase (RT)-PCR Diagnostic Panel

| | CDC 2019 Novel Coronavirus (2019-nCoV) | | |
|---|---|---|---|
| | Real-Time Reverse Transcriptase | | |
| | (RT)-PCR Diagnostic Panel | | |
| Detect test | Positive | Negative | Total |
| Positive | 43 | 0 | 43 |
| Negative | 2 | 62 | 64 |
| Total | 45 | 62 | 107 |

| | |
|---|---|
| PPA: | 95.6% (43/45) (95% CI: 85.2%-98.8%) |
| NPA: | 100% (62/62) (95% CI: 94.2%-100.0%) |
| Invalid Rate | 12.3% (15/122) (95% CI: 7.6%-19.3%) |
| (after retest): | |

TABLE 17
Detect Test Performance Against the
Roche cobas SARS-CoV-2 RT-PCR Test

| | Roche cobas SARS-CoV-2 RT-PCR Test | | |
|---|---|---|---|
| Detect test | Positive | Negative | Total |
| Positive | 43 | 0 | 43 |
| Negative | 1 | 63 | 64 |
| Total | 44 | 63 | 107 |

| | |
|---|---|
| PPA: | 97.7% (43/44) (95% CI: 88.2%-99.6%) |
| NPA: | 100% (63/63) (95% CI: 94.3%-100.0%) |
| Invalid Rate | 12.3% (15/122) (95% CI: 7.6%-19.3%) |
| (after retest): | |

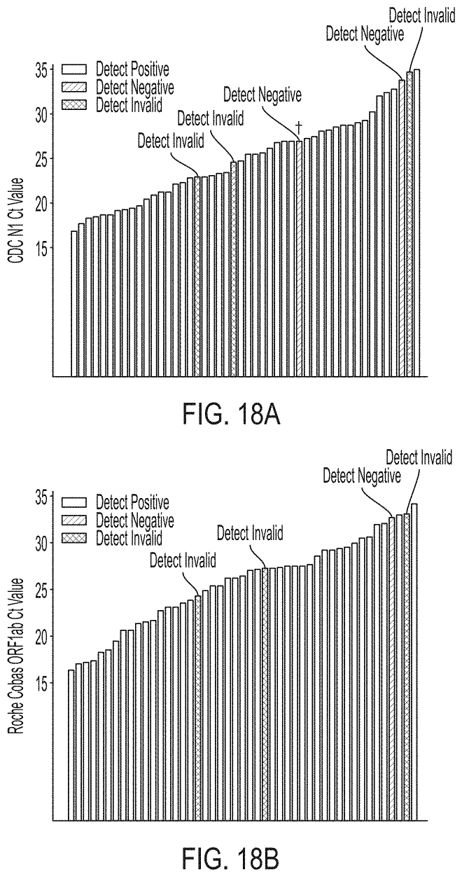

To further analyze the Detect test's PPA, FIGS. 18A-18B demonstrate the Detect test's results on samples that produced a SARS-CoV-2 positive result on the CDC assay (panel A), ordered by the CDC assay's N1 Ct value and on samples that produced a SARS-CoV-2 positive result on the cobas test, ordered by the cobas test's ORF1ab Ct value (panel B). Of the Detect test's two apparent false negatives (compared to the CDC Assay's result), one of them had a CDC N1 Ct value of 33.8, and the other had a CDC N1 Ct value of 27.0. The latter discrepant result is notable because the Detect test reliably produced a positive result for all other study samples with an CDC N1 Ct near that level and because the Roche cobas test also produced a negative result on this sample. Furthermore, this was the only sample for which the CDC assay's and the cobas test's results were discrepant. The Detect test's only apparent false negative (compared to the cobas test) had a cobas ORF1ab Ct value of 32.8.

vi. Notification of Public Health Authorities

Per the CARES Act Section 18115 regarding COVID-19 Pandemic Response, Laboratory Data Reporting, all sites were required to report the reference method testing results for all subjects enrolled in the study to the appropriate local or state health agency per their state requirements.

### Invalid Results Analysis

In this section, a summary of the invalid results analysis is presented. A breakdown of invalid results by operator is provided in Table 18. For the one hundred and twenty-two (122) patients enrolled, 12.3% (15/122) of the Detect test's results were invalid. Notably, 60% (9/15) of the Detect test's invalid results were obtained from tests run by a single operator (BNB), who was observed during a site visit on her first day using the Detect test not to have even looked at the Detect test instructions before beginning testing. Of this operator's nine (9) invalid test results, eight (8) were obtained in the first week of testing. Based on the data obtained in this study, a root cause analysis was conducted that determined that operator error was responsible for the majority of invalid results. When the Detect test was run by experienced operators, the invalid rate was less than 5%, over multiple kit lots, many operators, and hundreds of replicates. The invalid rate observed in this study is reflective of a “worst-case” scenario, in which the operator does not receive any instruction, watch instructional videos, or receive feedback upon producing several invalid results. The complete invalid results root cause analysis is presented in Example 2.

In the Detect test's protocol, after sample collection the operator stores the Collection Tube with its remaining sample in the refrigerator. In the event of an invalid result, the operator takes the stored Collection Tube out of the refrigerator, opens a new Detect test kit, and repeats the procedure, starting with the transfer pipette step as described elsewhere herein. The result of this retest—whether positive, negative, or invalid—is considered final.

Invalid results on a first test (before retesting) that were resolved to positive or negative results (after retesting) did not impact the efficacy of the Detect test because only the result of the retest was considered final. As demonstrated in Table 18, 63.4% (26/41) of the forty-one (41) invalid results obtained on the first test were resolved to either a positive or a negative result upon retesting. It is also noteworthy that the retesting procedure did not involve further interaction with the patient. As described above, the patient did not re-collect a new nasal sample in the event that a retest was required. As noted in Table 18, flex studies indicated that collected nasal samples remained stable for up to 4 hours even at room temperature, thereby providing the operator ample time to complete the retest procedure after an initial patient encounter. Therefore, the patient only received the final test result, and the retesting procedure did not require any additional interaction with the patient.

Invalid results that remained invalid after retest did not impact the NPA or PPA of the Detect test but were problematic in that a patient seeking a test result was unable to obtain one. The Detect test was designed—via inclusion of the RNase P control (described elsewhere herein)—to produce an invalid result instead of an incorrect (false negative) result in the event of improper test execution. There was an inherent tradeoff between the number of ways (and therefore the likelihood) that a test could produce an invalid result and the test's PPA. The Detect test—designed for widespread use at the point of care—was designed to maximize PPA by utilizing a stringent control strategy (RNase P). This ensured that both positive and negative test results could be communicated to patients with confidence.

Table 18 supports the above claim, demonstrating that there was no relation between an operator's invalid rate and the operator's performance (PPA and NPA)—the operator with the most invalid results (BNB) had no false negatives and no false positives.

TABLE 18
Invalid Rate and Performance by Operator

| | | Invalid Rate | | Invalid Rate | | PPA | | | | NPA | | | |
|---|---|---|---|---|---|---|---|---|---|---|---|---|---|
| Site | Operator | (after retest) | | (first test) | | CDC | | cobas | | CDC | | cobas | |
| ADP | K-D | 50.0% | (1/2) | 50.0% | (1/2) | N/A (0/0) | | | | 100.0% (1/1) | | | |
| PPM | PLC | 13.0% | (3/23) | 17.4% | (4/23) | 71.4% | (5/7) | 83.3% | (5/6) | 100.0% | (13/13) | 100.0% | (14/14) |
| SFR | ADV | 4.3% | (1/23) | 43.5% | (10/23) | 100.0% (19/19) | | | | 100.0% (3/3) | | | |
| VHP | BNB* | 19.1% | (9/47) | 46.8% | (22/47) | 100.0% (9/9) | | | | 100.0% (29/29) | | | |
| | MEB | 3.7% | (1/27) | 14.8% | (4/27) | 100.0% (10/10) | | | | 100.0% (16/16) | | | |
| Total | All | 12.3% | (15/122) | 33.6% | (41/122) | 95.6% | (43/45) | 97.8% | (43/44) | 100.0% | (62/62) | 100.0% | (63/63) |
| Total | All (excluding | 8.0% | (6/75) | 25.3% | (19/75) | 94.4% | (34/36) | 97.1% | (34/35) | 100.0% | (33/33) | 100.0% | (34/34) |
| | BNB) | | | | | | | | | | | | |

*On a site visit to VHP during operator BNB's first day participating in the study, it was noted that BNB did not read the Quick Reference Instructions (included with the Detect test kit) before beginning to use the Detect test. She immediately began making critical errors in the test procedure. It was therefore unsurprising that Detect tests run by BNB gave more invalid test results than all of the other operators in the study combined.

### Performance Around Limit of Detection (LoD)

The PPM site participating in protocol CS-1206-01 also participated in the Performance around LoD study (CS-1206-02). The testing was performed by three (3) untrained intended use operators.

Spiked swabs were presented to the operators in a manner that appeared similar to swabs collected under protocol CS-1206-01. Pooled nasal matrix was generated following the same methodology used for the LoD study as described elsewhere herein.

Swabs were coded to ensure that all operators were blinded to their identity and SARS-CoV-2 status. An operator was handed one swab at a time to test. Swabs were tested throughout the course of the day. The reproducibility of the Detect test was demonstrated in the hands of multiple intended users. Each intended use operator tested a minimum of three (3) SARS-CoV-2 positive and three (3) SARS-CoV-2 negative samples.

The sample types are described in Table 19, below. Table 20 summarizes the study design.

TABLE 19
Performance around LoD Samples (CS-1206-02 Sample Matrix)

| | | Virus Concentration and |
|---|---|---|
| Sample Type | Virus | Swab Preparation |
| <2x LoD positive | BEI heat- | 50 μL contrived low positive |
| contrived | inactivated virus | nasal matrix pipetted onto an |
| | (Lot #70034991) | unused swab (15,750 |
| | | copies/swab - 1.91x LoD) |
| Negative | None | 50 μL of contrived negative |
| contrived | | nasal matrix pipetted onto an |
| | | unused swab |

TABLE 20
Performance around LoD Study Design

| Daily Sample | Site 1 | | | |
|---|---|---|---|---|
| Panel | IUO 2 | IUO 2 | IUO 3 | Total |
| COVID Positive | 3 | 3 | 4 | 10 |
| COVID Negative | 3 | 3 | 4 | 10 |
| Total Samples Tested | 6 | 6 | 8 | |

IUO = Intended Use Operator

External quality controls (positive and negative, as described elsewhere herein) were also performed by each operator in accordance with the CS-1206-01 protocol.

Each intended use operator tested the samples in a random fashion. Testing was performed over the course of two days. The site ultimately tested twenty-two (22) samples (eleven (11) SARS-CoV-2 positive and eleven (11) SARS-CoV-2 negative). Overall test results and individual operator results are shown below in Tables 19 and 20.

There were no significant differences in the observed sensitivity of the Detect test with low positive samples between operators. The study demonstrated that untrained intended use operators were able to accurately perform and interpret the Detect test near the LoD.

TABLE 21
Performance around LoD Results

| Sample Category | Count | Percent Agreement and 95% CI |
|---|---|---|
| Positive | 11/11 | 100.0% (95% CI: 74.1-100.0%) |
| Negative | 10/11* | 90.9% (95% CI: 62.3%-98.4%) |

*One sample was invalid and was not recovered through retest.

TABLE 22
Performance around LoD Operator to Operator Results

| Sample Category | | Positive | Negative |
|---|---|---|---|
| Site | Operator | Count | Count |
| | | (% Agreement) | (% Agreement) |
| PPM | JSP | 3/3 (100.0%) | 3/4 (75.0%) |
| | | (95% CI: 43.9%-100.0%) | (95% CI: 30.1%-95.4%) |
| | PLC | 4/4 (100.0%) | 4/4 (100.0%) |
| | | (95% CI: 51.0%-100.0%) | (95% CI: 51.0%-100.0%) |
| | ZRS | 4/4 (100.0%) | 3/3 (100.0%) |
| | | (95% CI: 51.0%-100.0%) | (95% CI: 43.9%-100.0%) |

### Flex Studies

To assess the robustness of the Detect test to deviations in assay performance, flex studies were conducted under a variety of potential environmental conditions or likely user errors. All flex studies were conducted with both contrived negative and contrived low positive (2×LoD) samples with five replicates each. Pooled nasal matrix was generated by the same methods described in the LoD study.

a. Delay in Reaction Set-Up after Sample Collection

To test the effect of a delay between sample collection and reaction set-up that might occur during a retest, five contrived positive and five contrived negative samples were generated and run immediately through the Detect test, while the sample remnants were stored at 4° C. and retested at 1, 2, and 4 hours. All tests with delays in reaction preparation showed the expected results, with the exception of one positive replicate which gave an invalid result at the 2-hour time-point but was recovered at the 4-hour time-point. Overall, the data indicated that the assay was robust to delays in reaction set-up that might occur during a retest procedure.

To test the effect of improper storage of the sample remnant, the study was repeated with sample remnant storage at ambient temperature (26.7° C.), and the robustness of the assay to delays of up to 4 hours was confirmed.

TABLE 23
Effect of Delays between Sample Collection and
Reaction Set-up on the Detect test

| | | Negative | Positive |
|---|---|---|---|
| Delay between | | Samples | Samples |
| sample collection | Storage temperature | #Negative/ | # Positive/ |
| and reaction set-up | during delay | # tested | # tested |
| 0 min | 4° C. (as specified in | 4/5* | 4/5* |
| 1 hr | Detect QRG) | 5/5 | 5/5 |
| 2 hr | | 5/5 | 4/5* |
| 4 hr | | 5/5 | 5/5 |
| 0 min | Ambient, 26.7° C. | 5/5 | 5/5 |
| 1 hr | (deviation from | 5/5 | 5/5 |
| 2 hr | QRG) | 5/5 | 5/5 |
| 4 hr | | 5/5 | 5/5 |

*One replicate was invalid.

b. Delay in Reaction Incubation

The effect of delay in incubation of assembled reactions was tested by assembling reactions from contrived positive and contrived negative samples and delaying incubation for 5 minutes, 30 minutes, 1 hour, or 2 hours. Reactions were held either at ambient room temperature (19.6-23.6° C.) or in a thermal chamber at 40° C. to mimic extreme conditions. The Detect test tolerated delays of up to 2 hours at standard room temperature and 1 hour at 40° C., showing it was robust to delays prior to incubation.

TABLE 24
Effect of Reaction Incubation Delays on the Detect test

| | | Negative | Positive |
|---|---|---|---|
| Delay between | Storage | Samples | Samples |
| reaction assembly | temperature | #Negative/ | # Positive/ |
| and incubation | during delay | # tested | # tested |
| 5 min | Ambient | 5/5 | 5/5 |
| 30 min | (19.6-23.6° C.) | 5/5 | 5/5 |
| 1 hr | | 5/5 | 5/5 |
| 2 hr | | 5/5 | 5/5 |
| 5 min | Extreme | 5/5 | 5/5 |
| 30 min | (40° C.) | 5/5 | 5/5 |
| 1 hr | | 5/5 | 5/5 |
| 2 hr | | 4/5* | 5/5 |

*One replicate was invalid.

c. Delay in Test Interpretation

To assess the effect of a delay in test interpretation upon the observed results, five contrived positive samples, five contrived negative samples, and five no-template controls (NTCs) were run, and the Readers were interpreted after 2 minutes, 5 minutes, 10 minutes (QRG interpretation time), 45 minutes, and 60 minutes. The results did not change at the various tested time points, indicating that the test tolerated both early and late interpretation. A second set of samples showed that the test was also robust to a 60-minute time delay at high temperature and humidity (40.8-41.3° C. and 87-89% relative humidity).

TABLE 25
Effect of Test Interpretation Delays on the Detect test

| | Storage temperature | | |
|---|---|---|---|
| | and | Negative | Positive |
| | relative humidity | Samples | Samples |
| Delay before | (RH) | #Negative/ | # Positive/ |
| interpretation | during delay | # tested | # tested |
| 2 min | Ambient | 5/5 | 5/5 |
| 5 min | (19.8° C., 27% RH) | 5/5 | 5/5 |
| 10 min (QRG) | | 5/5 | 5/5 |
| 45 min | | 5/5 | 5/5 |
| 60 min | | 5/5 | 5/5 |
| 60 min | | 5/5 | 5/5 |

*Temperature and humidity in the thermal incubation chamber fluctuated slightly during the 60-minute incubation period.

d. Variation in Sample Transfer Volume

In the Detect test, the fixed volume transfer Pipette provided in the Detect test kit was used to transfer 250 μL of the collected sample to the Warmer Tube for reaction incubation. The tolerance of the Detect test to variation in sample volume transfer was assayed by using a using a laboratory pipette to transfer 200 μL, 250 μL, or 350 μL of sample from the Collection Tube to the Warmer Tube for five contrived positive and five contrived negative samples. All of the 200 μL samples gave the expected result, but the negative samples showed sensitivity to the 350 μL volume transfer. The study was repeated at 300 μL and the 250 μL reference volume, and all samples gave the expected result, indicating that the test could tolerate 50 μL variation in volume transfer in either direction. The test instructions contained careful step-by-step illustrations of proper transfer Pipette use to mitigate the risk of user error resulting in invalid test results.

TABLE 26
Effect of Reaction Volume Variation on the Detect test

| Reaction volume | Negative Samples | Positive Samples |
|---|---|---|
| transferred | #Negative/# tested | # Positive/# tested |
| 200 μL | 5/5 | 5/5 |
| 250 μL | 10/10 | 10/10 |
| 300 μL | 5/5 | 5/5 |
| 350 μL | 1/5 (4 invalid) | 5/5 |

*Because the transfer pipette provided in the Detect test kit in the tested embodiment delivered a fixed volume of sample, sample volume transfers were not tested at extreme volumes 0.5× or 2× the specified reaction volume.

The results shown herein indicated that the sample could be stored for up to 4 hours at room temperature (as opposed to refrigeration). Similarly, flex study 2 demonstrated that the Detect test tolerated keeping the prepared reaction at 40° C. for an hour prior to incubation. In these studies, humidity was not tested separately because the liquid samples were kept in sealed Collection Tubes or Warmer Tubes with limited headspace. In the case of results interpretation, where humidity could plausibly affect the wicking of the sample onto the lateral flow strip, readout delay was tested at 40° C. and 87-89% RH, which showed no effect upon test results interpretation.

### Example 2—Analysis of Invalid Results

An analysis of data from the clinical study of Example 1 was conducted to evaluate the relative frequency of invalid result presentations.

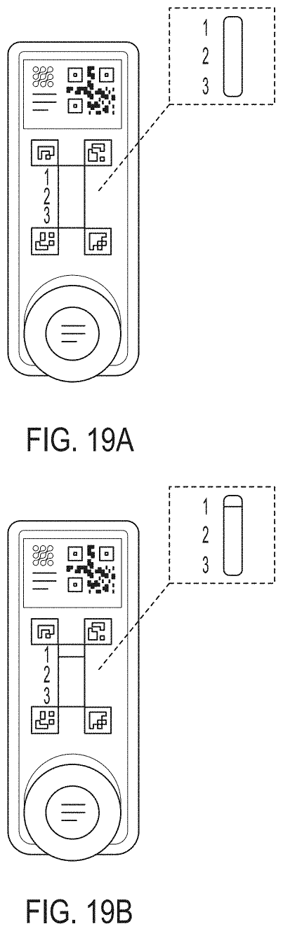

There were two distinct presentations of invalid results that occurred with the Detect test. First, as shown in FIG. 19A, there was the “No Flow” invalid test result, where no bands appeared on the Reader's lateral flow strip. This was due to a mechanical failure of liquid to wick onto the lateral flow strip from the Warmer Tube. Second, as shown in FIG. 19B, there was the “No Sample Processing Control” invalid test result, where only band 1 (the Reader Check Control) appeared on the Reader's lateral flow strip. This indicated that liquid successfully wicked through the lateral flow strip, but neither SARS-CoV-2 nor the human control target RNase P was detected. This invalid result occurred either because the RNase P amplification failed or because the RNase P amplification occurred, but there was a failure during readout that caused band 3 (the Sample Processing Control band) not to be visible. The presence of band 3 (the Sample Processing Control) was only required for the result to be valid when band 2 was not present. Other band patterns that would have indicated an invalid result were rarely observed.

1. Invalid Results Failure Modes and Effects Analysis (FMEA)

From observing untrained operators in action, reasoning from first principles about various aspects of the product design, and receiving feedback from subjects in usability studies, a list of potential causes that could result in an invalid result were proposed to serve as the basis for an FMEA Analysis (Table 27). This FMEA served as the foundation for the invalid results root cause analysis.

TABLE 27
Invalid Results FMEA - For each failure mode, the likelihood of causing an invalid result
(severity), the likelihood of occurring (occurrence), and the likelihood of an observer
being able to detect this failure mode (detection) are presented on a 1-10 scale.

| Row | Failure Mode | Potential Cause | Potential Effect | Severity | Occurrence | Detection |
|---|---|---|---|---|---|---|
| 1 | No Flow | Under-torqued | Too little | 5 | 6 | 9 |
| | (vapor | Cap: Operator | reaction | | | |
| | lock) | applies Test | liquid reaches | | | |
| | | Cap with too | lateral flow strip | | | |
| | | little torque | (LFS) to initiate | | | |
| | | | flow | | | |
| 2 | No Sample | | No reaction | 6 | 2 | 2 |
| | Processing | | liquid reaches | | | |
| | Control (pre | | LFS to initiate | | | |
| | flow) | | flow, but | | | |
| | | | sufficient | | | |
| | | | Dropper buffer | | | |
| | | | reaches LFS, | | | |
| | | | thereby causing | | | |
| | | | flow control | | | |
| | | | band to | | | |
| | | | develop, but no | | | |
| | | | other bands | | | |
| 3 | No Flow | Operator | Reaction liquid | 2 | 8 | 2 |
| | (banging | bangs Reader | scatters and | | | |
| | Reader) | on Table | sticks to tube | | | |
| | | | surfaces due to | | | |
| | | | surface tension | | | |
| 4 | No Flow | Operator | Too little buffer | 8 | 4 | 5 |
| | (dropper | does not | liquid reaches | | | |
| | dispense) | empty enough | LFS to initiate | | | |
| | | buffer from | flow | | | |
| 5 | No Sample | Dropper | Bands that | 4 | 3 | 1 |
| | Processing | Operator | develop are too | | | |
| | Control | dispenses | faint to be | | | |
| | (Dropper | Dropper | visible | | | |
| | dispense) | liquid onto | | | | |
| | | Reader's | | | | |
| | | Chimney | | | | |
| | | Walls | | | | |
| | | Operator | | | | |
| | | dispenses | | | | |
| | | Dropper | | | | |
| | | outside of | | | | |
| | | Reader's | | | | |
| | | Chimney | | | | |
| 6 | No Sample | Operator | Too much | 4 | 1 | 1 |
| | Processing | dispenses too | buffer liquid | | | |
| | Control | much Dropper | reaches LFS, | | | |
| | (Dropper | liquid into | flow only | | | |
| | dispense) | Reader's | contains buffer | | | |
| | | Sample Pad | (no reaction | | | |
| | | Dropper was | liquid) and does | | | |
| | | filled above | not contain | | | |
| | | tolerable limit | amplicons. | | | |
| 7 | No Sample | Operator does | Lyophilized | 9 | 6 | 3 |
| | Processing | not properly | reagent bead | | | |
| | Control (lyo | invert and | insufficiently | | | |
| | bead | shake tube | resuspended, | | | |
| | resuspension) | after applying | causing master | | | |
| | | Test Cap | mix | | | |
| | | | component(s) to | | | |
| | | | deviate from | | | |
| | | | tolerable range. | | | |
| 8 | No Sample | Operator skips | Too much | 4 | 4 | 2 |
| | Processing | the wrist snap | liquid is trapped | | | |
| | Control | step, or | on tube walls | | | |
| | (liquid | executes it | and/or lyo cage, | | | |
| | collection at | incorrectly | causing master | | | |
| | tube bottom) | (ending on the | mix | | | |
| | | upstroke, or | component(s) to | | | |
| | | holding tube | deviate from | | | |
| | | upside down) | tolerable range. | | | |
| 9 | No Flow | | Too much | | | |
| | (liquid | | liquid is trapped | | | |
| | collection at | | on tube walls | | | |
| | tube bottom) | | and/or lyo cage, | | | |
| | | | decreasing | | | |
| | | | liquid volume | | | |
| | | | so that surface | | | |
| | | | tension cannot | | | |
| | | | be overcome | | | |
| | | | when tube is | | | |
| | | | pierced in the | | | |
| | | | Reader or | | | |
| | | | insufficient | | | |
| | | | reaction liquid | | | |
| | | | is present to | | | |
| | | | initiate flow | | | |
| 10 | No Sample | Operator | Master mix | 5 | 2 | 1 |
| | Processing | transfers too | component(s) | | | |
| | Control (too | much liquid | and/or template | | | |
| | much/too | from | concentrations | | | |
| | little volume | Collection | deviate from | | | |
| | transferred | Tube to | tolerable range | | | |
| | with pipette) | Warmer | | | | |
| | | Tube with | | | | |
| | | transfer | | | | |
| | | pipette | | | | |
| 11 | No Sample | RNase P | RNase P | 9 | 2 | 2 |
| | Processing | expression | amplification | | | |
| | Control | level in | fails because | | | |
| | (sample | certain subset | RNase P | | | |
| | diversity) | of population | template is | | | |
| | | is below | present in | | | |
| | | assay's LoD | concentrations | | | |
| | | | below assay's | | | |
| | | | LoD | | | |
| 12 | No Sample | User doesn't | RNase P | 6 | 2 | 1 |
| | Processing | collect enough | amplification | | | |
| | Control | nasal material | fails because | | | |
| | (improper | | RNase P | | | |
| | sample | | template is | | | |
| | collection) | | present in | | | |
| | | | concentrations | | | |
| | | | below assay's | | | |
| | | | LoD | | | |
| 13 | No Sample | Nasal | RNase P | 9 | 2 | 2 |
| | Processing | inhibitor | amplification | | | |
| | Control | levels too high | fails because of | | | |
| | (sample | in certain | high levels of | | | |
| | diversity in | subset of | nasal inhibitors | | | |
| | nasal | population | | | | |
| | inhibitors) | | | | | |
| 14 | No Sample | Warmer | RNase P | 10 | 1 | 1 |
| | Processing | malfunction | amplification | | | |
| | Control | causes | fails because | | | |
| | (Warmer | temperature | RNase P | | | |
| | malfunction) | protocol to | template is | | | |
| | | deviate from | present in | | | |
| | | tolerable | concentrations | | | |
| | | range | below assay's | | | |
| | | | LoD | | | |
| 15 | No Sample | Incomplete | RNase P | 8 | 3 | 1 |
| | Processing | tube insertion | amplification | | | |
| | Control | prevents | fails because | | | |
| | (improper | reaction from | reaction | | | |
| | tube insertion | reaching | temperature is | | | |
| | in Single- | correct | too low | | | |
| | Well | temperature | | | | |
| | Warmer) | | | | | |

2. Clinical Study Invalid Results Analysis

a. Breakdown of Invalid Results Presentations

An analysis of the clinical study data was conducted to evaluate the relative frequency of “No Flow” and “No Sample Processing Control” invalid result presentations (Table 28). The fact that 19.5% (8/41) of first test invalid results were “No Flow” invalids provided direct evidence for the occurrence of some combination of root causes 1, 3, and 4 in Table 27 above. It also clearly demonstrated that the majority of invalid results presented as “No Sample Processing Control.”

TABLE 28
CS-1206-01 Invalid Results Breakdown

| First Test Invalid Result Type | | Retest Invalid Result Type | |
|---|---|---|---|
| | No Sample | | No Sample |
| No Flow | Processing Control | No Flow | Processing Control |
| 19.5% (8/41) | 80.5% (33/41) | 6.7% (1/15) | 93.3% (14/15) |

Failures pertaining to sample collection or composition were recognized as being unlikely to significantly contribute to the invalid test results rate. In addition, the fact that 63.4% (26/41) of invalid results obtained on a first test were resolved to a valid result upon retesting (Table 29) provided evidence against the proposed root causes that pertained to a failure in sample collection (Table 27, row 12) or a characteristic of the sample itself (Table 27, rows 11 and 13). If there had been an issue with the sample, it should have manifested on the retest, which was run off of the same sample used for the first test.

TABLE 29
CS-1206-01 Invalid Result Resolution Upon Retesting

| Valid Retest Results | Invalid Retest Result |
|---|---|
| 63.4% (26/41) | 36.6% (15/41) |

Invalid results rate is operator dependent, but PPA and NPA are not.

An analysis was conducted to evaluate the invalid test results rate obtained by each of the five rapid diagnostic test operators that participated in the study (Table 30). There was a large discrepancy observed between operators: the lowest invalid test results rate was 3.7% (1/27) and the highest invalid test results rate was 19.1% (9/47). The data demonstrates that there is no relation between an operator's invalid test results rate and the operator's performance (PPA and NPA) the operator with the most invalid results (BNB) had no false negatives and no false positives.

TABLE 30
Invalid Rate and Performance by Operator

| | | Invalid Rate | | Invalid Rate | | PPA | | | | NPA | | | |
|---|---|---|---|---|---|---|---|---|---|---|---|---|---|
| Site | Operator | (after retest) | | (first test) | | CDC | | cobas | | CDC | | cobas | |
| ADP | K-D | 50.0% | (1/2) | 50.0% | (1/2) | N/A (0/0) | | | | 100.0% (1/1) | | | |
| PPM | PLC | 13.0% | (3/23) | 17.4% | (4/23) | 71.4% | (5/7) | 83.3% | (5/6) | 100.0% | (13/13) | 100.0% | (14/14) |
| SFR | ADV | 4.3% | (1/23) | 43.5% | (10/23) | 100.0% (19/19) | | | | 100.0% (3/3) | | | |
| VHP | BNB* | 19.1% | (9/47) | 46.8% | (22/47) | 100.0% (9/9) | | | | 100.0% (29/29) | | | |
| | MEB | 3.7% | (1/27) | 14.8% | (4/27) | 100.0% (10/10) | | | | 100.0% (16/16) | | | |
| Total | All | 12.3% | (15/122) | 33.6% | (41/122) | 95.6% | (43/45) | 97.8% | (43/44) | 100.0% | (62/62) | 100.0% | (63/63) |
| Total | All (excluding | 8.0% | (6/75) | 25.3% | (19/75) | 94.4% | (34/36) | 97.1% | (34/35) | 100.0% | (33/33) | 100.0% | (34/34) |
| | BNB) | | | | | | | | | | | | |

A site visit was conducted to observe the intended use operators at the Texas (VHP) clinical site. Of the two operators at the Texas site, one (MEB) diligently read the rapid diagnostic test kit instructions (e.g., “Quick Reference Instructions”) before beginning testing and was observed to follow all steps of the rapid diagnostic test correctly on the 5 tests that were performed during the site visit. The second operator (BNB) did not read the Quick Reference Instructions before beginning testing and was observed to make several critical errors in the 6 tests that were performed during the site visit. Over the course of the study, operator BNB had an invalid test result rate more than five times higher than operator MEB. Over time, operator BNB improved from her baseline of 34.85% (8/23) invalid test results during the first week to a 4.2% (1/24) invalid test results rate for the remaining three weeks of the study (Table 31). This suggests that some operators may experience a learning curve.

TABLE 31
CS-1206-01 Invalid Results Obtained by Operator
BNB in First Week of Testing and Thereafter

| | Invalid Rate | Invalid Rate |
|---|---|---|
| Dates of Testing Performed | (after retest) | (first test) |
| Dec. 10, 2020-Dec. 16, 2020 | 34.8% (8/23) | 60.9% (14/23) |
| Dec. 17, 2020-Jan. 4, 2021 | 4.2% (1/24) | 33.3% (8/24) |

3. Further Invalid Results Root Cause Analysis

When run correctly, the rapid diagnostic test of the present invention produces an invalid rate of less than 5%. Quality Control testing of a random sample of 20 rapid diagnostic test kits from two different lots was performed by Detect employees on fresh nasal swabs collected by presumed negative subjects enrolled under IRB at the Detect Connecticut site. On each Test Kit lot, a 5% (1/20) invalid rate was obtained before retesting. The retesting procedure was not performed in these tests, because their purpose was to evaluate manufacturing quality and estimate defect rates.

In the analytical studies (Limit of Detection, Flex Testing, Interfering Substances) described in elsewhere herein, the invalid rate was 4.0% (16/404). Of these, 12 of the 16 invalid results were produced in conditions that were intentionally outside of the rapid diagnostic test's intended operating conditions. Eight invalids occurred in the presence of high concentrations of biotin and another four invalids occurred when the transfer pipette step transferred 40% more liquid (350 μL) than the nominal amount (250 μL). These internal studies were run by experienced test operators.

i. Usability Study with Untrained Healthcare Professionals

A usability study was run at Detect headquarters in Guilford CT on Jan. 6, 2020 with two healthcare professionals (representative of typical point of care operators) who had never before run the rapid diagnostic test. A Detect human factors engineer observed each operator perform all steps of the rapid diagnostic test and recorded observations. The operators were untrained and ran the rapid diagnostic test, given only the Quick Reference Instructions, on fresh nasal swabs collected from subjects enrolled from the local population (under IRB). Both operators obtained an invalid test results rate (after retesting) of 4.5% (1/22). Operator 1 obtained a first test invalid results rate (before retesting) of 13.6% (3/22), and the other operator obtained a first test invalid results rate of 27.3% (6/22).

TABLE 32
Result from Usability Study on Jan. 6, 2021-Jan. 7, 2021

| Operator | Invalid Rate (after retest) | Invalid Rate (first test) |
|---|---|---|
| Operator 1 | 4.5% (1/22) | 13.6% (3/22) |
| Operator 2 | 4.5% (1/22) | 27.3% (6/22) |
| Total | 4.5% (2/44) | 20.5% (9/44) |

A further analysis was performed to reveal the root causes responsible for the invalid test results obtained. Rapid diagnostic test kit Readers that display the “No Sample Processing Control” band pattern could be caused either because the RNase P amplification failed, or because despite RNase P amplification being successful there was a failure upon readout to produce a visible Sample Processing Control band (band 3, FIGS. 19A-19B). The former failure mode can be caused only by errors in the test steps 1-6, and the latter failure more can be caused by errors only by errors in the test steps 7-9. Therefore, determining which of these two failure modes is responsible for the invalid test result is useful for narrowing down the root cause of the invalid test result.

In light of the above, each rapid diagnostic test kit's Reader that produced an invalid test result was analyzed using a standard laboratory procedure to determine whether that sample's RNase P amplification was successful or not (Table 32 above). For operator 1, it was determined that RNase P amplification was successful for both of the samples that initially produced a “No Sample Processing Control” invalid test result, thereby indicating that the failure must have occurred in one or more of test steps 7-9. This conclusion is further corroborated by the observations of this operator running those samples—which noted that the operator did not make any noticeable error in test step execution. It is therefore likely that the causative error was related to the operator's use of the Dropper (Table 27, row 5 or row 6), which, as described in the FMEA analysis, is difficult to detect from observation of the operator. For operator 2, it was determined that RNase P amplification failed for all six of the samples that initially produced a “No Sample Processing Control” invalid test result, thereby indicating that the failure must have occurred in one or more of test steps 1-6. This conclusion is further corroborated by the observations of this operator running those samples—which noted that for three of the samples, the operator made the critical error of performing the pipetting step twice, and the other 3 samples, the operator incorrectly performed the lyophilized reagent bead resuspension. These data provide clear evidence for the occurrence of the root causes described in row 7-10 in the FMEA analysis (Table 27).

TABLE 33
Root Cause Analysis from Usability Study on Jan. 6, 2021-Jan. 7, 2021

| | Invalid Test Results Root Cause Analysis | | |
|---|---|---|---|
| | “No Sample Processing | “No Sample Processing | |
| | Control”: RNase P | Control”: RNase P | |
| Operator | Amplification Failed | Amplification Successful | “No Flow” |
| Operator 1 | 0.0% (0/3) | 66.7% (2/3) | 33.3% (1/3) |
| Operator 2 | 100.0% (6/6) | 0.0% (0/6) | 0.0% (0/6) |
| Total | 66.7% (6/9) | 22.2% (2/9) | 11.1% (1/9) |

Operator error in the pipette transfer, dropper dispense, and lyophilized reagent bead resuspension steps was responsible for the majority of invalid test results obtained. Taken together, the data presented in this section demonstrate that the majority of invalid test results were due to operator error in one of three steps: the transfer pipette step, the lyophilized reagent bead resuspension step, and the Dropper step. This conclusion is also corroborated by observations made during the visit to one of the sites (VHP) that participated in the clinical study.

4. Risk Analysis Summary from Invalid Results

Invalid results on a first test (before retesting) that are resolved to positive or negative results (after retesting) do not impact the efficacy of the rapid diagnostic test because only the result of the retest is considered final. As demonstrated in Table 29, 63.4% (26/41) of the forty-one (41) invalid results obtained on the first test were resolved to either a positive or a negative result upon retesting. It is also noteworthy that the retesting procedure does not involve further interaction with the patient; the patient does not re-collect a new nasal sample in the event that a retest is required. As noted, the flex studies indicate that collected nasal samples remain stable for up to 4 hours even at room temperature, thereby providing the operator ample time to complete the retest procedure after an initial patient encounter. Therefore, the patient only receives the final test result and the retesting procedure does not require any additional interaction with the patient.

Invalid results that remain invalid after retest do not impact the NPA or PPA of the rapid diagnostic test but are problematic in that a patient seeking a test result was unable to obtain one. The rapid diagnostic test was designed—via inclusion of the RNase P control—to produce an invalid result instead of an incorrect (false negative) result in the event of improper test execution. There is an inherent tradeoff between the number of ways (and therefore the likelihood) that a test can produce an invalid result and the test's PPA. The rapid diagnostic test designed for widespread use at the point of care—was designed to maximize PPA by utilizing a stringent control strategy (RNase P). This ensures that both positive and negative test results can be communicated to patients with confidence.

The primary risk to the patient is that a patient seeking a test result may have a delayed test result due to reprocessing the specimen (retest). The impact associated with invalid results is limited to a delay in diagnosis. The rapid diagnostic test kit's Instructions for Use will include a warning to the operator to notify the patient immediately of an invalid test result and request that the patient quarantine, in an abundance of caution, until the test can be repeated. Precautionary measures of quarantine will mitigate the exposure to others, if the patient with an invalid result is positive for SARS-CoV-19.

### Example 3—Detect Test Component Stability

In this Example, components of an exemplary diagnostic system were tested for stability. It is advantageous for components to be stable, as unstable components that may age during shipping or other forms of distribution, or while being stored prior to distribution or use, may be undesirable and produce unreliable test results.

### Stability of Test Kit Components

a. Contrived Sample Preparation

Contrived SARS-CoV-2 positive samples for stability studies were generated using pooled nasal matrix (see Limit of Detection (LoD) study description in Example 1 for detailed preparation method) with BEI heat-inactivated SARS-CoV-2 virus added at 2X LoD prior to pipetting onto unused swabs.

b. Reagent Stability

Experiments were conducted on certain individual components of the Detect test, such as the lyophilized reagent bead contained in the Test Cap and the Collection Buffer contained in the Collection Tube, to determine their stability. The components used in these single-component stability studies were stored in production packaging exactly replicating storage conditions in a rapid diagnostic test kit. To replicate the effect of being stored at certain temperatures over a length of time (e.g., 5 to 6 months), accelerated stability approximations were used.

c. Basis for Accelerated Stability Approximations

Because the aging process for many materials is mediated by chemical reactions that are thought to be temperature dependent, raising the temperature at which material is stored accelerates these reactions according to the relationship described in the Arrhenius equation. A conservative first approximation of this effect is that every 10° C. increase in temperature roughly doubles the reaction rate and therefore also doubles the rate of the aging process. In the studies below, for example, incubation at 45° C.—which is 15° C. above the high end of the rapid diagnostic test kit's intended storage range—resulted in an acceleration factor of 2^{1.5} or 2.83× acceleration.

d. Collection Tube Accelerated Stability

Collection Tubes filled with Collection Buffer (as described herein) were assembled into rapid diagnostic test kit packaging and stored at 45° C. for 53 days and 65 days to simulate 5- and 6-month durations at 30° C., respectively. After the incubation period at 45° C. was complete, the Collection Tubes were stored at controlled room temperature (15° C.-30° C.) until testing. Collection Buffer subjected to 5 or 6 months of simulated aging was tested alongside Collection Buffer stored at −20° C. and Collection Buffer prepared fresh to serve as controls for the impact of exposure to elevated temperatures. The aged Collection Buffer was tested using contrived positive samples at 2×LoD and contrived negative samples, generated as laid out in the “Contrived Sample Preparation” section, above. No apparent performance impact was observed after 45° C. storage for up to 65 days, approximating 6 months of real-time stability at 30° C. Results are shown in Table 34, below.

TABLE 34
Accelerated Stability Data for the Collection Tube

| | | | Expected Result/Replicates Tested | | |
|---|---|---|---|---|---|
| Simulated | | | Contrived | Contrived | |
| Duration | | | Positives | Negatives | NTCs |
| (30° C. | Actual | Storage | # Positive/ | # Negative/ | # Invalid/ |
| storage) | Duration | Condition | # Tested | # Tested | #Tested |
| 5 months | 53 days | 45° C. | 4/4 (100%) | 3/3 (100%) | 0/1 (0%)* |
| (150 days) | | | | | |
| 6 months | 65 days | 45° C. | 4/4 (100%) | 3/3 (100%) | 0/1 (0%)* |
| (184 days) | | | | | |
| Controls | | −20° C. | 1/1 (100%) | 1/1 (100%) | 1/1 (100%) |
| | | Fresh | 1/1 (100%) | 1/1 (100%) | 1/1 (100%) |
| | | (baseline) | | | |

*Two of the No Template Controls showed human control gene contamination.

e. Test Caps—Accelerated Stability

Test Caps, as described herein, (lyophilized reagent beads assembled into plastic caps and enclosed in foil pouches) were assembled into rapid diagnostic test kit packaging along with other rapid diagnostic test kit components and stored at 45° C. to simulate 3, 5, and 6 month durations at 30° C. Test Caps stored at −20° C. served as a control for the impact of exposure to elevated temperatures. The Test Caps were assayed with contrived positive samples only, as laid out in the “Contrived Sample Preparation” section, above. The samples were generated from fresh nasal swabs spiked with 16,500 copies/swab of SeraCare AccuPlex™ SARS-CoV-2 encapsulated RNA, 2× the LoD as determined with heat-inactivated SARS-CoV-2. No substantial performance impact was observed after 45° C. storage for up to 65 days, approximating 6 months of real-time stability at 30° C. Results are shown in Table 35, below.

TABLE 35
Accelerated Stability Data for the Test Cap

| | | | Expected Result/ | |
|---|---|---|---|---|
| | | | Replicates Tested | |
| | | | Contrived | |
| Simulated | | | Positives | NTCs |
| Duration (30° C. | Actual | Storage | # Positive/ | #Invalid/ |
| storage) | Duration | Condition | # Tested | #Tested |
| 3 months (93 | 33 days | 45° C. | 7/7 (100%) | — |
| days) | | | | |
| Controls (baseline) | | −20° C. | 3/3 (100%) | 1/1 (100%) |
| 5 months (150 | 53 days | 45° C. | 7/7 (100%) | — |
| days) | | | | |
| Controls (baseline) | | −20° C. | 2/3 (67%) | 1/1 (100%) |
| 6 months (184 | 65 days | 45° C. | 7/7 (100%) | — |
| days) | | | | |

| | | | |
|---|---|---|---|
| Controls (baseline) | −20° C. | 3/3 (100%) | 0/1 (0%)* |

*One of the No Template Controls showed human control gene contamination.

f. Test Cap—Elevated Temperature, Real-Time Stability

Test Caps, as described herein, were stored at 30° C. and removed for stability testing approximately every 2 weeks, starting with a 2-week timepoint. After the incubation period at 30° C. was complete, the Test Caps were assayed on the same day or were stored at controlled room temperature (15° C.-30° C.) until testing. Test Caps stored at −20° C. served as a control for the impact of exposure to elevated temperatures. For the 2- and 4-week data points, fresh swabs were used for negative samples, and contrived positives were generated by spiking 10 copies/μL of heat-inactivated SARS-CoV-2 directly into reactions from fresh swab samples. Thereafter, the nasal matrix pooling and delivery method referenced in the “Contrived Sample Preparation” section above was employed with pooled nasal matrix spiked with heat-inactivated SARS-CoV-2 virus at 2×LoD for contrived positive samples and pooled nasal matrix for contrived negative samples. No substantial performance impact was observed after 30° C. or −20° C. storage for up to 42 days, approximating 6 weeks of real-time stability at 30° C. Results are shown in Table 36, below.

TABLE 3
Real-Time Stability Data for the Test Cap at 30° C.

| | | Expected Result/Valid Replicates Tested | | |
|---|---|---|---|---|
| | | Contrived | Contrived | |
| | | Positive | Negative | NTCs |
| Approximate | Storage | (# Positive/ | (# Negative/ | (# invalid/ |
| Duration | Condition | # Tested) | # Tested) | #Tested) |
| 2 weeks | 30° C. | 18/18 (100%) | 18/19 (95%)* | 3/3 (100%) |
| | | 2 invalid | 1 invalid | |
| Control | −20° C. | 19/19 (100%) | 16/17 (94%)* | |
| | | 1 invalid | 3 invalid | |
| 4 weeks | 30° C. | 20/20 (100%) | 19/20 (95%)* | 3/3 (100%) |
| Control | −20° C. | 20/20 (100%) | 19/20 (95%)* | |
| 6 weeks** | 30° C. | 20/20 (100%) | 20/20 (100%) | 3/3 (100%) |
| Control | −20° C. | 20/20 (100%) | 20/20 (100%) | |

*One contrived negative sample gave a SARS-CoV-2 positive result.
**Contrived sample strategy updated to nasal matrix pooling strategy.

g. Reader Real-Time Stability for Lateral Flow Strip in Different Housing

The Reader's stability is dictated by that of the lateral flow strip that it contains. The contract manufacturer that produces the lateral flow strip conducted real-time stability studies (in compliance with ISO 23640:2015) that demonstrated that the lateral flow strips, when stored in a plastic cassette, have an 18 month shelf-life when stored between 10° C. and 25° C. This 18-month shelf-life is attested to by the manufacturer's Certificate of Conformance that accompanies each shipment of lateral flow strips.

The storage environment for the lateral flow strip in the rapid diagnostic test kit packaging (inside of the Reader, which is placed in the foil rapid diagnostic test kit pouch containing desiccant) is distinct, but similar to that used in the manufacturer's stability study. Therefore, the stability of the Reader is expected to be similar to that claimed by the lateral flow strip manufacturer.

h. Sample Stability

As described in Example 12, sample stability—after elution into the Collection Tube—has been validated for up to 4 hours, for samples stored either refrigerated or at room temperature.

### Stability of Test Kit

In embodiments where a rapid diagnostic test kit of the present invention is manufactured and distributed as described herein, the test kit as a whole is tested for stability. Unstable kits that may age during shipping or other forms of distribution, or while being stored prior to distribution, may be undesirable and produce unreliable test results.

Detailed below is an experiment which tests reagent stability under commercial conditions in the rapid diagnostic kit as a whole.

a. Contrived Sample Preparation

Contrived SARS-CoV-2 positive samples for the proposed stability studies will be generated using pooled nasal matrix (see Limit of Detection (LoD) study description in Example 12 for detailed preparation method) with BEI heat-inactivated SARS-CoV-2 virus added at 2×LoD prior to pipetting onto unused swabs.

b. Functional Testing

The following conditions and replicates will be used to evaluate stability for each condition or timepoint:
Contrived Positive Tests: 10 unopened rapid diagnostic test kits will be used with 10 contrived positive Swabs at 2×LoD prepared from a single positive nasal matrix pool.
Contrived Negative Tests: 10 unopened rapid diagnostic test kits will be used with 10 contrived negative Swabs prepared from a single negative nasal matrix pool.
No Template Control (NTC) Tests: 5 rapid diagnostic test kits will be used with 5 unused swabs.
Sample preparation and test execution will be carried out in nominal controlled room temperature and humidity (15-30° C., 20-60% RH).
All tests will use the Multi-Well or Single-Well Warmer according to the corresponding user manual.

c. Shelf-Life Stability

Real-time shelf-life stability at 30° C. is assessed according to the following schedule for one or more lots according to the details described in the Contrived Sample Preparation and Functional Testing sections described above:
Testing Timepoints (time after Test Kit assembly): <2 weeks (baseline), 1 month, 3 months, 6 months, 9 months, 12 months, and 15 months.
Storage Conditions: The Test Kit is intended to be stored at ambient room temperature. For stability testing, the Test Kits will be stored at 30° C.

Acceptance Criteria for Each Timepoint:
No more than 2 failures to detect the RNase P Sample Processing Control out of the ten contrived negatives tested.
No more than a single false negative (i.e., failure to detect SARS-CoV-2 in a contrived positive sample) out of the ten contrived positives tested.
None of the NTCs shows detection of the RNase P Sample Processing Control or SARS-CoV-2. If any does, testing for the timepoint must be repeated.

Shelf life Determination: The shelf-life for the Test Kit will be set based on the next-to-last condition to pass the acceptance criteria set above.

d. Shipping Stability

The shipping stability of the rapid diagnostic test will be assayed by subjecting three groups of rapid diagnostic test kits to different environmental conditioning tests following guidelines from the American Society for Testing and Materials (ASTM D4332-14). Afterward, each group of rapid diagnostic test kits will be subjected to a physical challenge meant to simulate air (intercity) and motor freight (local) distribution stresses according to ASTM D4169-16, Dist. Cycle 13. For all tests, individual rapid diagnostic test kits will be packaged in their larger shipping carton as would be the case during actual shipping and distribution. The three environmental conditions are as follows:

Condition A: −30° C.; uncontrolled relative humidity for 24 hours

Condition B: +40° C.; 90% relative humidity for 24 hours

Condition C: +60° C.; 15% relative humidity for 24 hours

The impact of the environmental conditioning and physical challenge to packaging will be assessed according to the following standards:

Visual Inspection (ASTM F1886/F1886M-16)

Gross Leak Testing (ASTM F2096-11)

Seal Strength Testing (ASTM F88/F88M-15)

The impact of thermal conditioning and physical stresses on rapid diagnostic test kit functional performance will be assessed according to the functional testing strategy described above.

Acceptance Criteria for Each Shipping Condition:
No more than 2 failures to detect the RNase P Sample Processing Control out of the ten contrived negatives tested.
No more than a single false negative (i.e., failure to detect SARS-CoV-2 in a contrived positive sample) out of the ten contrived positives tested.
None of the NTCs shows detection of the RNase P Sample Processing Control or SARS-CoV-2. If any does, the assessment will be repeated using additional kits from the physically stressed shipping carton.

Actions: Packaging, shipping conditions, and customer handling guidelines may be modified or added based on the results of the shipping stability study to ensure proper function of the rapid diagnostic test kits.

### Example 4—Sensitivity and Specificity of Primers and Probes

Candidate primers and probes were screened to meet the condition that not a single position in their binding sites was polymorphic in the existing published genome sequences. First, a multiple alignment of the N gene from published SARS-CoV-2 genome sequences was constructed as follows.

All genomes from GISAID EpiCoV database meeting the following criteria were downloaded on 21 Mar. 2020 from gisaid.org: “exclude low coverage”, “complete” and “human” host. They were co-aligned using the MAFFT multiple alignment program (alignment algorithm =“auto”). The N gene ORF was extracted from the multiple alignment manually, based on the multiple alignment coordinates of the N gene in the EPI_ISL_402125 sequence (ORF coordinates taken from GenBank annotations of this RefSeq genome sequence NCBI Accession: NC_045512.2). Aligned N gene ORFs were then removed from the multiple alignment if they contained one or more stretches of 10 or more consecutive “N” positions (ambiguous nucleotides), or one or more stretches of 100 or more “-” positions (alignment gaps). To ensure 100% inclusivity, during the downstream manual primer/probe screening process (using primer3), primers were only considered if every position they targeted was universally conserved in the resulting alignment.

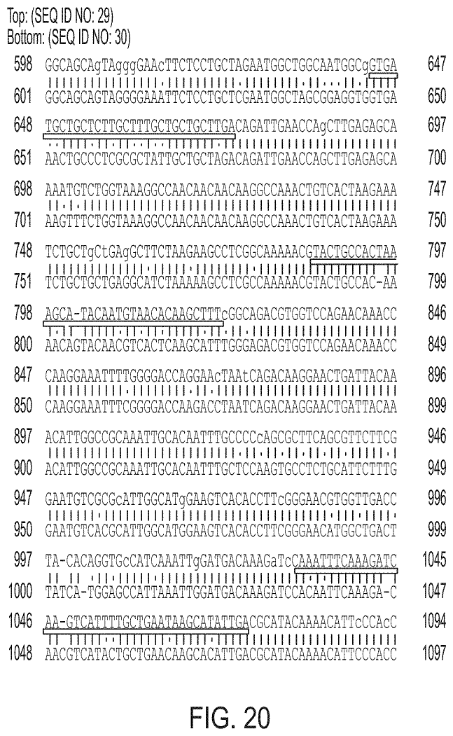

The most homologous genome to SARS-CoV-2 in terms of cross-reactivity is SARS-coronavirus (SARS-CoV-1), and so this was used as the initial cross-reactivity constraint in manually choosing primer-binding regions in the N gene ORF. To achieve this, the SARS-CoV-2 reference sequence EPI_ISL_402125 N gene ORF (encoded to highlight the universally conserved positions) was aligned with the SARS-CoV-1 NCBI RefSeq N gene ORF (extracted from the annotated GenBank file NC_004718.3, coordinates 28120-29388), using the EMBOSS Needle global pairwise alignment program. Three regions (“candidate regions 1-3”) in the resulting alignment were visually identified as potentially harboring primer-binding sites consisting of only universally conserved positions (within SARS-CoV-2 variants) while showing more than 20% divergence from the associated sites in the aligned SARS-CoV-1 sequence (FIG. 20; SARS-CoV-2 is the upper sequence, with uppercase characters for those positions universally conserved among SARS-CoV-2 variants).

The EPI_ISL_402125 N gene ORF sequence (encoded as described above) between candidate regions 1 and 2, and separately between candidate regions 2 and 3, was analyzed in primer3 with primer parameters recommended for efficient amplification, and the output was manually screened to identify forward and reverse primer pairs in which all targeted positions were universally conserved within SARS-CoV-2 and both primer binding-sites are more than 20% divergent to the aligned sites in the SARS-CoV-1 reference sequence. No suitable probe-binding sites could be identified that met the joint criteria of 100% inclusivity and less than 80% homology with SARS-CoV-1, so candidate probes were selected only on the criterion of 100% inclusivity. This was not expected to be problematic for expected functioning of the test, as the SARS-CoV-1 RNA sequence homologous to the probe was not expected to be reverse-transcribed and amplified by the primers. Moreover, there has not been a documented case of SARS-CoV-1 in several years.

Primers and probes were then screened for cross-reactivity with all other specific organisms noted to potentially be cross-reactive, along with potential microbial flora from the human respiratory tract and human sequences themselves, by querying them in an exhaustive blastn search against the NCBI nt database. Using the default parameters for a “short sequence” task, primers and probes were only selected if no homology ≥80% was detected with any other human-associated microorganisms or human sequences themselves.

In silico analysis demonstrated that the primers described herein do not exceed the homology threshold with any of the organisms listed as potentially cross-reactive. In silico analysis demonstrates that the only organism for which the probe exceeds the homology threshold is SARS-CoV-1. Cross-reactivity between the probe and SARS-CoV-1 is clinically irrelevant because there has not been a documented case of SARS-CoV-1 in several years. Moreover, probe cross-reactivity should not impact test performance, even in the highly unlikely presence of SARS-CoV-1 co-infection, as the SARS-CoV-1 RNA sequence homologous to the probe is not expected to be reverse-transcribed and amplified by the primers.

### Example 5—RPA Sample and Testing Conditions

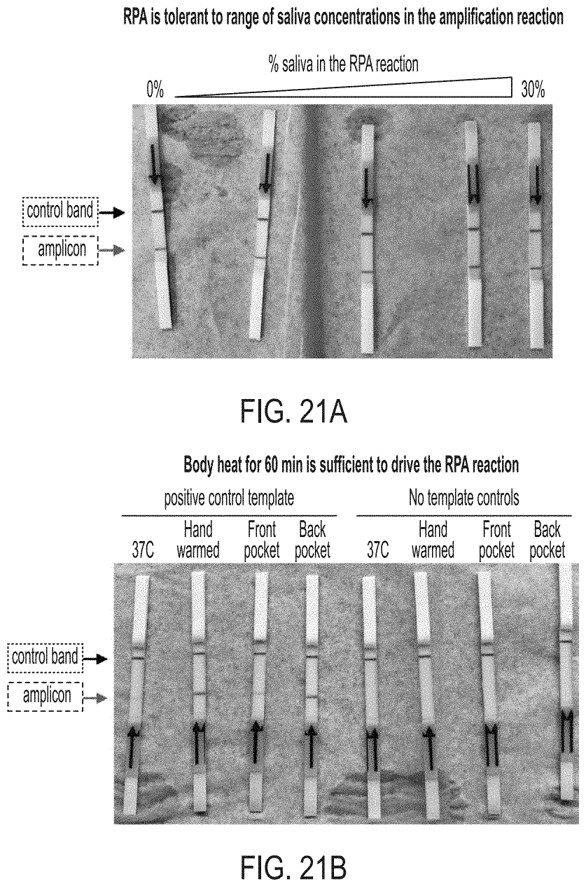

In this Example, the effects of saliva concentration and heat on RPA (using saliva samples) were investigated. First, RPA reactions were conducted using samples with varying levels of saliva concentration. As shown in FIG. 21A, both a test band and a control band were visible for all concentrations tested, demonstrating tolerance of the RPA-based diagnostic test to saliva concentrations ranging from 0% to 30%. Thus, this demonstrates the tolerance of the test to varying concentrations of a saliva sample.

Second, the RPA-based diagnostic test was performed using body heat for 60 minutes by placing a reaction tube in various locations on an individual's body (hand-warmed, front pant pocket, rear pant pocket), both with a positive control and without controls. As shown in FIG. 21B, all locations resulted in a readable lateral flow test strip.

### Example 6—Lateral Flow Test Detection of COVID-19

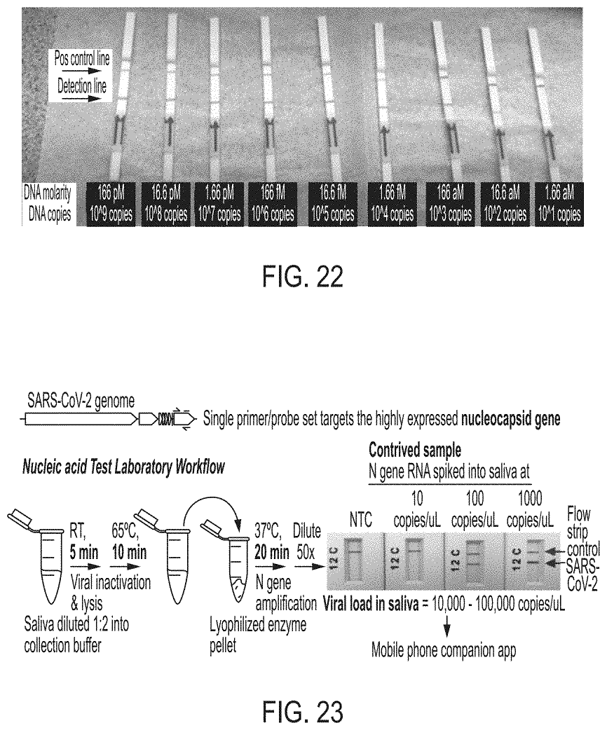

In this Example, an RPA-based diagnostic test was used to detect COVID-19 DNA in samples. As shown in FIG. 22, concentrations as low as 100 aM DNA were successfully detected using the RPA lateral flow test strips.

The experiment was repeated using readout devices described herein. Spiked samples were used, and amounts of COVID-19 RNA inputs of 13 aM were detected.

As illustrated in FIG. 21A, RPA is very tolerant of saliva samples. In one experiment, lyophilized RPA mixture was resuspended in 100% saliva, which was sufficient for running the test (data not shown).

### Example 7—Lateral Flow Strip Quality Control (Saliva Sample Concentration)

An experiment was undertaken to evaluate the sensitivity of an exemplary lateral flow strip tests with respect to different concentrations of viral load. As shown in FIG. 23, a single primer/probe set targeting the SARS-Cov-2 nucleocapsid gene was used. Contrived saliva samples (spiked with nucleocapsid gene RNA at 10, 100, or 1000 copies per μL) were diluted 1:2 in collection buffer. The samples were incubated at room temperature for five minutes and then heated to 65° C. for 10 minutes for inactivation and lysis. The resulting solution was added to a new tube comprising a lyophilized enzyme pellet comprising amplification enzymes. The pellet was dissolved in the solution, and the tube was heated to 37° C. for 20 minutes to amplify the nucleocapsid gene DNA. Samples were then diluted 50-fold and run on lateral flow test strips. As shown in FIG. 23, viral loads as low as 100 copies/μL were detected.

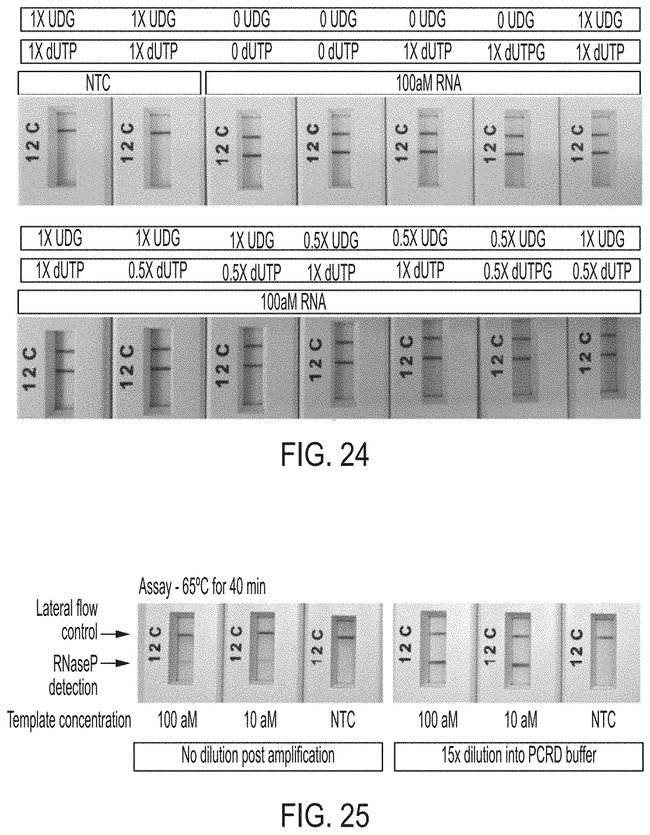

Further, different concentrations of UDG and dUTP were examined. As shown in FIG. 24, all of the combinations tested (0 UDG, 0.2×UDG, 1 UDG, 0 dUTP, 0.5×dUTP, 1 dTUP) showed a visible SARS-CoV-2 line. Concentrations of 100 aM RNA were used.

### Example 8—Lateral Flow Strip LAMP Test

An experiment was undertaken to illustrate LAMP and lateral flow strip testing. LAMP primers against human RNase P (a sample-positive control) were used to amplify samples (100 aM or 10 aM). Known human RNase P primers labeled with biotin/FITC or biotin/DIG were used (Curtis et al., 2018), and samples were incubated with the primers from 40 minutes at 65° C. The processed samples were then either diluted and run on the lateral flow strips, or undiluted and run directly on the lateral flow strips. As shown in FIG. 25, RNase P amplified by LAMP, even down to initial concentrations of 10 aM, was visible on the lateral test strips without dilution.

### Example 9—Colorimetric Detection of COVID-19 RNA

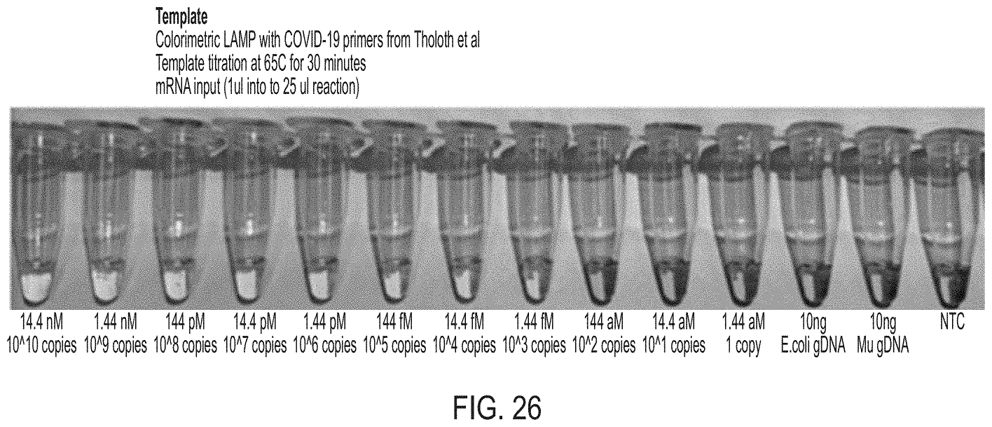

Colorimetric RT-LAMP was performed using COVID-19 primers (Toloth, et al.). The template was titrated at 65° C. for 30 minutes, and 1 μl of COVID-19 mRNA was added to a 25 μL reaction. The results, shown in FIG. 26, illustrate that the procedure successfully detected approximately 1 fM of COVID-19 RNA in solution.

The experiment was repeated with multiplexing reagents for lateral flow testing with a positive control amplicon.

### Example 10—Colorimetric LAMP Experiments

Colorimetric assays were used to monitor LAMP reactions in real time for 60 minutes. The signal was found to be prominent at 30 minutes and to peak at 40 minutes (data not shown). The results were the same when DNA templates or RNA templates were run. Six LAMP primer sets were examined, and detection was visible in the 10-100 aM range (data not shown).

Various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing,” “involving,” and variations thereof, is meant to encompass the items listed thereafter and additional items.

The terms “approximately,” “substantially,” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

1. A diagnostic system, comprising:
a sample-collecting component;
one or more nucleic acid amplification reagents, wherein the one or more nucleic acid amplification reagents comprise a first primer directed to a first target nucleic acid and labeled with a first label; and
a readout device configured to detect the presence of the first target nucleic acid.

2. The diagnostic system of claim 1, wherein the first target nucleic acid is a nucleic acid of SARS-CoV-2.

3. The diagnostic system of claim 1, wherein the readout device comprises a lateral flow strip, wherein the lateral flow strip comprises a first test line comprising a first capture reagent configured to bind to the first label.

4. The diagnostic system of claim 1, wherein the readout device comprises a chimney configured to receive a reaction tube, wherein the chimney is in fluidic communication with the lateral flow strip.

5. The diagnostic system of claim 1, further comprising a first reaction tube and/or a second reaction tube.

6. The diagnostic system of claim 5, further comprising a cap comprising one or more reagents, wherein the cap is configured to fit on an open end of the first reaction tube and/or the second reaction tube.

7. The diagnostic system of claim 6, wherein the one or more reagents form at least a portion of a lyophilized bead.

8. The diagnostic system of claim 6, wherein the one or more reagents comprise one or more nucleic acid amplification reagents.

9. The diagnostic system of claim 8, wherein the one or more nucleic acid amplification reagents comprise one or more LAMP reagents.

10. The diagnostic system of claim 6, wherein the at least one reagent comprises UDG.

11. The diagnostic system of claim 1, wherein each component of the diagnostic test is stable at 30° C. for at least 5 months.

12. The diagnostic system of claim 1, further comprising a heater.

13. The diagnostic system of claim 1, wherein the one or more nucleic acid amplification reagents comprise a first primer directed to a second target nucleic acid sequence and labeled with a first label, wherein the second target nucleic acid sequence is different from the first target nucleic acid sequence.

14. The diagnostic system of claim 13, wherein the second target nucleic acid is a nucleic acid of an influenza virus.

15. A diagnostic system, comprising:
a sample-collecting component configured to collect a sample;
one or more isothermal nucleic acid amplification reagents, wherein the one or more isothermal nucleic acid amplification reagents comprise a first primer directed to a first target nucleic acid; and
a readout device configured to detect the first target nucleic acid when the concentration of the first target nucleic acid in the sample is about 5 genomic copies per μL or more.

16. The diagnostic system of claim 15, wherein the first target nucleic acid is a nucleic acid of SARS-CoV-2, and wherein the system has a positive percent agreement of at least 95% and/or a negative percent agreement of at least 95% compared to the CDC 2019 Novel Coronavirus Real-Time RT-PCR Diagnostic Panel.

17. The diagnostic system of claim 15, wherein the system has an invalid rate of about 15% or less.

18. The diagnostic system of claim 15, wherein the sample-collecting component is a self-administrable sample-collecting component.

19. A diagnostic method, comprising:
collecting a sample from a subject;
performing an isothermal nucleic acid amplification reaction configured to amplify a first target nucleic acid; and
detecting the presence or absence of the first target nucleic acid in the sample within 75 minutes.

20. The diagnostic method of claim 19, wherein the subject performs the collecting step.

21. The diagnostic method of claim 19, wherein the first target nucleic acid is a nucleic acid of SARS-CoV-2 or an influenza virus.

22. The diagnostic method of claim 19, wherein collecting the sample comprises swabbing an anterior nares region of at least one nostril of the subject.
